(19) United States
(12) Patent Application Publication
(10) Pub. No.: US 2023/0420110 A1
(43) Pub. Date: Dec. 28, 2023

(54) METHODS FOR OBJECTIVE ASSESSMENT, RISK PREDICTION, MATCHING TO EXISTING MEDICATIONS AND NEW METHODS OF USING DRUGS, AND MONITORING RESPONSES TO TREATMENTS FOR MOOD DISORDERS

(71) Applicants: Indiana University Research and Technology Corporation, Bloomington, IN (US); The United States of America as represented by the Department of Veterans Affairs, Washington, DC (US)

(72) Inventor: Alexander B. Niculescu, III, Indianapolis, IN (US)

(21) Appl. No.: 18/037,457

(22) PCT Filed: Nov. 18, 2021
(86) PCT No.: PCT/US2021/059935

§ 371 Date: May 17, 2023

Related U.S. Application Data
(60) Provisional application No. 63/115,405, filed on Nov. 18, 2020.

(51) Int. Cl. G16H 20/70; G16H 20/10; G16H 50/30; G16H 50/70; G16H 70/20; G16B 25/10

(52) U.S. Cl. CPC G16H 20/70; G16H 20/10; G16H 50/30; G16H 50/70; G16H 70/20; G16B 25/10

(57) ABSTRACT

Methods for objective and precise assessment, risk prediction, monitoring of disease course and response to treatment, and precise matching to existing, and to new method of use repurposed drugs, in mood disorders, such as for patients with depression or bipolar disorder. These methods are based on an objective analysis of specific biomarker panels, as well as on the integration of the biomarker panel data with clinical measures of mood, life satisfaction, psycho-socio-demographic risk factors, and clinical history severity. These methods provide a foundation for precision medicine for mood disorders.

### PRIORITY CLAIM

This application claims the benefit of U.S. provisional patent application No. 63/115,405, filed on Nov. 18, 2020, which is incorporated herein by reference in its entirety.

### GOVERNMENTAL SUPPORT

This invention was made with government support under OD007363 awarded by the National Institutes of Health and the CX000139 merit award by the Veterans Administration. The government has certain rights in the invention.

### FIELD

This invention generally relates to the methods of assessment, risk prediction, matching to treatments, and monitoring of response to treatment in mood disorders, through precision medicine.

### BACKGROUND

Mood disorders are disabling and, unfortunately, highly prevalent, affecting up to one in four individuals in their lifetime. Depression, which is characterized by overall depressed mood, is the leading cause of disability in the United States for people in the prime productive and reproductive ages of 15 to 44. Elevated moods are characterized by mania or hypomania, and the cycling between depressed and manic moods can be known as a bipolar mood disorder. Mood disorders are also present and often co-morbid with other psychiatric disorders.

Mood disorders are traditionally diagnosed via a physical examination combined with a mental health evaluation. Due to their reliance on self-reporting or a brief clinical impression, these mental health evaluations are not always reliable in forming an accurate diagnosis of the patient. A convergence of methods assessing a patient's internal subjective feelings and thoughts, along with external ratings of actions and behaviors, are used de facto in clinical practice to assess mood and diagnose clinical mood disorders. This subjective approach is insufficient, and lags behind assessment, risk prediction, and targeted therapies that are effectively used in other medical specialties. There are no current objective methods for measuring, scoring, or effectively and consistently diagnosing mood disorders. Further, when a mood disorder diagnosis is reached via a traditional mental health evaluation, the treatments available are not equally effective in all patient populations, and suffer from the same lack of reliability in follow-up efficacy measurement. Current methods of matching of individuals to treatments are not precise or personalized. This lack of objective diagnosis methodology and lack of matching of treatments when a diagnosis is reached, coupled with a perceived societal stigma associated with a mood disorder diagnosis, results in a chronic underdiagnosed and sub-optimal treatment of mood disorders.

### SUMMARY

Mood disorders, including depression and bipolar disorder, are traditionally diagnosed and monitored via subjective mental health evaluations. Additionally, the treatments available for mood disorders are not equally effective in all patient populations. The present disclosure provides methods for improved clinical diagnosis, treatment, and monitoring of mood disorders, through precision medicine. As opposed to traditional subjective mental health evaluations, these methods use an objective analysis of specific blood biomarker panels to provide enhanced assessment, risk prediction, targeted therapeutics, and monitoring for patients with depression and/or bipolar disorder.

The present disclosure provides a blood test (as well as tests using other types of biological samples from the patient) for the individualized assessment of depression or bipolar disorder, and a number of methods for utilizing this blood test for individualized assessment and treatments. The blood test uses one or more original panels of blood biomarkers to generate a patient-specific score, percentile ranking, and a traffic-light-type risk call or scoring determination for depression or bipolar disorder. The patient-specific score is generated based on particular methods for specific weighting of each biomarker.

The present disclosure further provides the use of these blood tests to generate a patient-specific profile that is used to match the patient with existing drugs used in clinical depression or bipolar disorder care, to identify the known therapeutics that are the most efficacious for the specific patient, on the basis of their biomarker expression levels.

The present disclosure also provides the use of these blood tests to provide a patient-specific signature, that is compared with a drug database, to identify repurposed therapeutic agents for the treatment of the patient's depression or bipolar disorder, on the basis of their biomarker signature.

A first embodiment is a method for diagnosing and treating mood disorders, and optionally monitoring response to treatment in an individual in need thereof, comprising the step of: (a) measuring the expression levels and/or slope of at least one biomarker in a biological sample from an individual, wherein the biomarkers in a first panel of biomarker comprise one or more of: TMEM161B, GLO1, PRPS1, SMAD7, ANK3, OGT, CD47, GLS, TMEM106B, RPL3, FANCF, HNRNPDL, DOCK10, or CALM1, and the biomarkers in a second panel of biomarkers comprise one or more of: NRG1, OLFM1, SPECC1, SORT1, TPH1, GSK3B, MARCKS, NR3C1, and SLC6A4; (b) comparing the expression level or slope of the at least one biomarker measured in the sample to the expression level and/or slope of a matched biomarker determined in a clinically relevant population; (c) generating a score for the individual, wherein the score is determined by summarizing: the number of biomarkers in the first panel of biomarkers exhibiting increased expression and/or slope relative to the expression level and/or slope in the matched biomarkers determined in the clinically relevant population and the number of biomarkers in the second panel of biomarkers expressing decreased expression and/or slope relative to the expression level and/or slope in the matched biomarkers determined in the clinically relevant population; (d) diagnosing the individual as having a mood disorder, and/or an increased risk for developing a mood disorder risk based on the difference between the scores of the individual and the scores of the matched biomarkers in the clinically relevant population; and (e) treating the individual diagnosed with a mood disorder, and/or the individual diagnosed with an increased risk for developing a mood disorder. In some versions of the first embodiment, step a is a cross-sectional analysis and consists of measuring the expression level of at least 1 biomarker. In some versions of the first embodiment, step a is a longitudinal analysis and includes measuring the expression level(s) and slope(s) of at least 1 biomarker.

A second embodiment is the method of the first embodiment, wherein the treating step includes treating the individual diagnosed with mood disorder and/or diagnosed with an increased risk for developing a mood disorder with a treatment consistent with clinical practice guidelines.

A third embodiment is a method according to either the first or the second embodiments, wherein the treating steps include providing the individual with at least one therapeutic compound known to treat mood disorders.

A fourth embodiment is a method according to either the first or the second embodiments, wherein the treating steps include providing the individual with at least one therapeutic compound which is a repurposed compound.

A fifth embodiment is a method according to either the first, second, third, or fourth embodiments, wherein the treating steps include further including the steps of: monitoring the individual to determine if the treatment is efficacious, wherein the monitoring step includes obtaining at least one additional biological sample from the individual; determining the score of the at least one additional biological sample from the individual; and comparing the scores of the at least one additional biological sample to the scores of the individual determined before and after or during treatment.

A sixth embodiment is a method according to either the first, second, third, fourth, or fifth embodiments wherein the mood disorder is selected from the group consisting of depression or bipolar disorder.

A seventh embodiment is a method according to the first, second, third, fourth, fifth, or sixth embodiments wherein the score is determined by assigning a weighted coefficient to each biomarker based on the importance of each biomarker in assessing and predicting mood disorders and an increase in risk of developing a mood disorder.

The eighth embodiment is a method according to the first, second, third, fourth, fifth, sixth or seventh embodiments, wherein the biological sample is a tissue sample or a fluid, such as cerebrospinal fluid, whole blood, blood serum, plasma, saliva, or other bodily fluid, or an extract, fraction, or purification product thereof.

A ninth embodiment is a method according to the first, second, third, fourth, fifth, sixth, seventh or eighth embodiments wherein the biomarker expression level of the biomarker is determined in the biological sample by measuring a level of biomarker RNA or protein.

A tenth embodiment is a method according to the first, second, third, fourth, fifth, sixth, or seventh, eighth or ninth embodiments wherein the individual is treated with at least one compound selected from the list comprising: lithium, valproic acid, and other mood stabilizers; amoxapine, paroxetine, mirtazapine, buspirone, fluoxetine, amitriptyline, nortriptyline, trimipramine, and other antidepressants; clozapine, chlorpromazine, haloperidol, paliperidone, iloperidone, asenapine, cariprazine, lurasidone, quetiapine, olanzapine, risperidone, aripiprazole, brexpiprazole, and other antipsychotics; docosahexaenoic acid and other omega-3 fatty acids; diazepam and other anxiolytics; ketamine and other dissociants; and CBT or other psychotherapy treatments.

An eleventh embodiment is a method according to the first, second, third, fourth, fifth, sixth, seventh, eighth, ninth or tenth embodiments wherein: (a) the individual exhibiting changes in one or more of biomarkers: NRG1, PRPS1, CD47 is treated with at least one mood stabilizing compound; (b) the individual exhibiting changes in one or more of biomarkers: SLC6A4, DOCK10, NRG1, CD47 is treated with at least one antidepressant compound; c) the individual exhibiting changes in one or more of biomarkers: GLO1, SLC6A4, CD47, GLS, HNRNPDL, is treated with at least one of the following compounds: docosahexaenoic acid and other omega-3 fatty acids; and (d) the individual exhibiting changes in one or more of biomarkers: NRG1, CD47, GLS, is treated with at least one antipsychotic compound.

A twelfth embodiment is a method according to the first embodiment, wherein the treating step includes administering to the individual at least one compound selected from the group consisting of: an isoflupredone, trichostatin A, dubinidine, ciprofibrate, pioglitazone, tropine, an adiphenine, saquinavir, chlorogenic acid, pindolol, lansoprazole, xamoterol, methanthelinium bromide, asiaticoside, an estradiol, methacholine, carteolol, chlorcyclizine, atracurium besylate, Chicago Sky Blue 6B, enoxacin, a levobunolol, 15-delta prostaglandin J2, pirinixic acid, NNC 55-0396 dihydrochloride, nadolol, MLN4924, U0126, amcinonide, iopanic acid, rosuvastatin and therapeutically acceptable salts thereof.

A thirteenth embodiment is a method according to the first embodiment wherein the individual is diagnosed with depression, when the expression levels of at least one of the biomarkers in the panel comprising: (a) TMEM161B, GLO1, PRPS1, SMAD7, CD47, GLS, FANCF, HNRNPDL, and DOCK10, in the biological sample of the individual are increased relative to the expression level of matched biomarkers determined in a clinically relevant population; and (b) NRG1, OLFM1, and SLC6A4, wherein the expression level of the biomarker(s) in the biological sample of the individual is decreased relative to the expression level of matched biomarkers determined in a clinically relevant population.

The fourteenth embodiment is a method according to the ninth embodiment, wherein the therapeutic is one or more of a repurposed drug selected from the group consisting of: an isoflupredone, trichostatin A, dubinidine, ciprofibrate, pioglitazone, tropine, an adiphenine, saquinavir, chlorogenic acid, pindolol, lansoprazole, xamoterol, methanthelinium bromide, asiaticoside, an estradiol, methacholine, a carteolol, chlorcyclizine, NNC 55-0396 dihydrochloride, nadolol, MLN4924, U0126, amcinonide, iopanic acid, and rosuvastatin.

A fifteenth embodiment is a method according to the first embodiment, wherein the individual is diagnosed with bipolar disorder, and the expression level of at least one of the biomarkers in a panel comprising: (a) TMEM161B, PRPS1, GLS, RPL3, and DOCK10, wherein the expression level of the biomarker(s) in the biological sample of the individual is increased relative to the expression level of matched biomarkers determined in a clinically relevant population, and (b) the expression level of at least one of the biomarkers in a panel comprising: NRG1, and SLC6A4, in the biological sample of the individual is increased relative to the expression level of matched biomarkers determined in a clinically relevant population.

A sixteenth embodiment is a method according to the eleventh embodiment, wherein the therapeutic is one or more of a new method of use/repurposed drugs selected from the group consisting of: atracurium besylate, Chicago Sky Blue 6B, enoxacin, levobunolol, 15-delta prostaglandin J2, ciprofibrate, pirinixic acid, an isoflupredone, and trichostatin A. In some aspects of the embodiment, the method includes using drugs known to treat the targeted condition. In some aspects of the embodiment, the method includes using repurposed drugs to treat the targeted condition.

A seventeenth embodiment is a method for monitoring response to treatment of a mood disorder and determining treatment efficacy in an individual, comprising the steps of: (a) measuring an expression level of at least one biomarker in at least 2 biological samples from the individual and comparing the measured expression levels to an expression level of a matched biomarker determined in a clinically relevant population, wherein the at least one biomarker is from a first panel, comprising: TMEM161B, GLO1, PRPS1, SMAD7, ANK3, OGT, CD47, GLS, TMEM106B, RPL3, FANCF, HNRNPDL, DOCK10, and CALM1, and/or measuring the expression level of at least one biomarker in at least 2 biological samples from the individual and comparing the measured expression levels to the expression level of a matched biomarker determined in a clinically relevant population, wherein the at least one biomarker is from a second panel comprising: NRG1, OLFM1, SPECC1, SORT1, TPH1, GSK3B, MARCKS, NR3C1, and SLC6A4, wherein the expression level of the one or more biomarkers in the biological sample is decreased, and wherein at least one of the at least two biological samples is collected before the individual is treated for a mood disorder and at least one of the at least two biological samples is collected after the individual is treated for a mood disorder; (b) calculating a score for the at least one biomarker in the biological samples, by summing: the number of biomarkers in the first panel exhibiting an increase in expression level relative to the expression of the biomarker determined in a clinically relevant population, and/or the number of biomarkers in the second panel exhibiting an decrease in expression level relative to the expression of the biomarker determined in a clinically relevant population; and (c) determining that said treatment(s) is effective if the score of the panel of biomarker(s) in the sample collected after treatment is lower than the score of at least one of the at least two biological samples collected before treatment.

An eighteenth embodiment is a method of assessing and treating mood disorders in an individual in need thereof, comprising: calculating combined biomarkers and clinical information Up-Mood based on the equation (Biomarker Panel Score)+(Clinical Risk Score)+(Mood Score)=Up-Suicide Score; wherein the Biomarker Panel Score is obtained as per the method of claim 1; wherein the Clinical Risk Score is calculated by summing up clinical risk factors of severity of illness; wherein the Mood Score is calculated by using a mood-rating scale; assessing the level of mood disorder of the individual by comparing the individual's Up-Mood Score to a reference Up-Mood Score; administering a treatment for mood to the individual when the individual's Up-Mood Score is different than a reference Up-Mood Score; and monitoring the individual's response to a treatment for mood by determining changes in the Up-Mood Score after initiating a treatment.

A nineteenth embodiment is a method comprising assessing mood, anxiety, and/or psychosis in the individual who has mood disorders, using a computer-implemented method for assessing mood, anxiety, psychosis, or combinations thereof, the method comprising: (a) receiving patient psychiatric information including mood information, anxiety information, psychosis information, or combinations thereof, into the computer device, wherein each of the patient psychiatric information is represented by a quantitative rating; and (b) computing the subtype for the patient, based upon their psychiatric information quantitative ratings.

A twentieth embodiment is a method according to the nineteenth embodiment wherein the assessing of mood, anxiety, psychosis or combinations thereof in the individual who has a low mood disorder/depression, classifies the individual in one of the following subtypes of low mood disorder: high anxiety and low psychosis (anxious), high anxiety and high psychosis (combined), low anxiety and high psychosis (psychotic), low anxiety and low psychosis (pure low mood).

A twenty-first embodiment is the method according to the first embodiment wherein the at least one biomarker is selected from the group comprising: NRG1, SLC6A4, DOCK10, or combinations thereof, and are used in all individuals.

A twenty-second embodiment is the method according to the first embodiment, wherein, the at least one biomarker is selected from the group comprising NRG1, SLC6A4, DOCK10, MARCKS, or combinations thereof, and are used in males.

A twenty-third embodiment is the method according to the first embodiment, wherein, the at least one biomarker is selected from the group comprising NRG1, SLC6A4, GLS, PRPS1, ANK3, or combinations thereof, and are used in females.

A twenty-fourth embodiment is the method according to the first embodiment, wherein, the at least one biomarker is selected from the group comprising MARCKS, SLC6A4, or combinations thereof, and are used in males with bipolar disorder.

A twenty-sixth embodiment is the method according to the first embodiment, wherein, the at least one biomarker is selected from the group comprising TMEM106B, SMAD7, ANK3, SORT1, PRPS1, DOCK10, or combinations thereof, and are used in females with bipolar disorder.

A twenty-seventh embodiment is the method according to the first embodiment, wherein, the at least one biomarker is selected from the group comprising NRG1, CD47, MARCKS, NR3C1, SLC6A4, or combinations thereof, and are used in males with depression.

A twenty-eighth embodiment is the method according to the first embodiment, wherein, the at least one biomarker is selected from the group comprising GSK3B, OLFM1, OGT, or combinations thereof, and are used in females with depression.

A twenty-ninth embodiment is the method according to the first embodiment, wherein, the at least one biomarker is selected from the group comprising NRG1, SLC6A4, or combinations thereof, and are used in males with PTSD.

A thirtieth embodiment is the method according to the first embodiment, wherein, NRG1 is used in females with PTSD.

A thirty-first embodiment is the method according to the first embodiment, wherein, the at least one biomarker is selected from the group comprising PRPS1, CALM1, SPECC1, TPH1, DOCK10, OLFM1, MARCKS, RPL3, NRG1, GSK3B, GLS, or combinations thereof, and are used in males with psychotic disorders.

A thirty-second embodiment is the method according to the first embodiment, wherein, the at least one biomarker is selected from the group comprising MARCKS, RPL3, or combinations thereof, and are used in females with psychotic disorders.

A thirty-third embodiment is a method for assessing and mitigating mood disorders in a patient in need thereof, comprising: determining an expression level of at least a first panel of blood biomarkers or a second panel of blood biomarkers in a sample from a patient, where the first panel of blood biomarkers comprises TMEM161B, GLO1, PRPS1, SMAD7, ANK3, OGT, CD47, GLS, TMEM106B, RPL3, FANCF, HNRNPDL, DOCK10, or CALM1 and where the second panel of blood biomarkers comprises NRG1, OLFM1, SPECC1, SORT1, TPH1, GSK3B, MARCKS, NR3C1, and SLC6A4; identifying the patient having a mood disorder when the expression level of the blood biomarkers in the first panel is increased relative to a reference expression level, or, the expression level of the blood biomarkers in the second panel is decreased relative to a reference expression level; and administering to the patient identified as having a mood disorder a drug to treat the mood disorder.

A thirty-fourth embodiment is a method according to the thirty-third embodiment, where the identifying step further comprises comparing a biomarker panel score of the patient to a biomarker panel score of a reference.

A thirty-fifth embodiment are methods according to the thirty-third and thirty-fourth embodiments, where the mood disorder is at least one disorder from the group consisting of: depression, bipolar mood disorder, and mania.

A thirty-sixth embodiment is a method according to the thirty-fifth embodiment, where the mood disorder is depression, and the panel of biomarkers includes one or more of the following biomarkers: TMEM161B, GLO1, PRPS1, SMAD7, ANK3, OGT, NRG1, GLS, DOCK10, HNRNPDL, FANCF, CD47, RPL3, OLFM1, CALM1, TPH1, SPECC1, MARCKS, TMEM106B, SORT1, GSK3B, NR3C1, and SLC6A4.

A thirty-seventh embodiment is a method according to the thirty-sixth embodiment, where the drug administered to the patient is at least one drug selected from the group consisting of: antidepressants, mood stabilizers, and antipsychotics.

A thirty-eighth embodiment is a method according to the thirty-fifth embodiment, where the mood disorder is bipolar mood disorder, and the panel of biomarkers includes one or more of the following biomarkers: TTLL3, CREBBP, DRD3, CKB, TRPM6, and MORF4L2.

A thirty-ninth embodiment is a method according to the thirty-eighth embodiment, where at least one drug is selected from the group consisting of: antidepressants, mood stabilizers and antipsychotics.

A fortieth embodiment is a method according to the thirty-fifth embodiment, where the mood disorder is mania and the panel of biomarkers includes one or more of the following biomarkers: RPL3 and SLC6A4.

A forty-first embodiment is a method according to the fortieth embodiment, where the drug administered to the patient is at least one drug selected from the group consisting of: mood stabilizers and antipsychotics.

These and other applications of the compositions and methods of the disclosure will be readily apparent to those of skill in the art in view of the following detailed description of various aspects and embodiments of the disclosure and its discovery and practice.

### BRIEF DESCRIPTION OF THE DRAWINGS

The features and advantages of the disclosure will become more readily appreciated as the same become better understood by reference to the following detailed description when taken in conjunction with the accompanying drawings. The accompanying drawings are not meant to limit any of the methods described herein.

FIGS. 1A-1F depict the Prioritization and Validation of Biomarkers for Mood. FIG. 1A is a schematic diagram depicting flow of discovery, prioritization, and validation of blood biomarkers.

FIG. 1B is a discovery cohort longitudinal within subject analysis.

FIG. 1C is a schematic which exemplifies certain genes identified with an internal score of 2 and above having differential expression (DE) or absent-present (AP) differential gene expression in the discovery cohort.

FIG. 1D represents prioritization with Convergent Functional Genomics (CFG) for prior evidence of involvement in mood.

FIG. 1E illustrates blood biomarker validation in two independent cohorts of psychiatric patients with clinically severe depression and clinically severe mania.

FIG. 1F is a schematic diagram depicting flow of discovery, prioritization, and validation of blood biomarkers.

FIGS. 2A-2D depict the area under the curve (“AUC”) for various blood biomarkers. All markers are nominally significant p<0.05. The tables underneath the figures display the actual number of biomarkers for each group whose ROC AUC p-values (FIG. 2A-2C) and Cox Odds Ratio p-values (FIG. 2D) are at least nominally significant.

FIG. 2A is a bar graph showing Low State Predictions (SMS-7s≤40).

FIG. 2B is a bar graph showing Depression State Predictions (HAMD≥22).

FIG. 2C is a bar graph showing Depression Trait Predictions First Year.

In FIG. 2D, bar graphs show Depression Trait Predictions All Future Years. All markers are nominally significant p<0.05.

FIG. 3 is a schematic diagram depicting the overlap of blood biomarker expression for use in treating depression, bipolar disorder, and mania.

FIG. 4 is an example chart depicting a depression score report generated using the panel of the depression biomarkers BioM 12 Depression (n=12 genes, 13 markers), as well as RPL3 for mania risk.

FIG. 5A is a schematic diagram of Simplified Mood Scale-7 (SMS-7) Visual Analog Scale for Measuring Mood State.

FIG. 5B is a graph representing the Correlation between HAMD and SMS7 in the whole population (n=794 testing visits).

FIG. 6A is an illustration of Mood-Pheno Chipping Clustering of items of the SMS-7 Visual Analog-Scale for measuring mood state.

FIG. 6B is a plot of the correlation between Hopefulness and SMS7 in a population subset on which there was a visual analog scale for Hope (n=84 testing visits).

FIG. 7 is an illustration of a two-way unsupervised hierarchical clustering of low mood visits.

FIG. 8 is a display representing an interaction network for predictive blood biomarkers for low mood/depression/hospitalizations.

FIG. 9 is a visual representation of the pharmacogenomics for depression (BioM12).

### DETAILED DESCRIPTION

This detailed description contains parts under separate headings, which merely assist a reader with the headers not limiting the claims or methods. Accordingly, as would be apparent to the skilled artisan, disclosure in any part can be relevant to disclosure in any other part.

As used in the specification and claims, the singular form “a,” “an” and “the” include plural references unless the context clearly dictates otherwise.

Throughout this disclosure, the following abbreviations are used: “BP” means bipolar disorder; “MDD” means major depressive disorder; “SZA” means schizoaffective disorder; “SZ” means schizophrenia; “PSYCHOSIS” means schizophrenia and schizoaffective combined; “PTSD” means post-traumatic stress disorder; “DE” means differential expression; “VAS” means visual analog-scale; “AP” means Absent/Present; “NS” means Non-stepwise in validation; “CFG” means Convergent Functional Genomics; “M” refers to a male patient (i.e., a patient having an X and a Y chromosome); and “F” refers to a female patient (i.e., a patient having two X chromosomes), “I” means increased; ‘D” means decreased; “hosp” means hospitalizations; “PBMC” means peripheral blood mononuclear cells; “ASD” autism spectrum disorder; and “AMY-SZ” means amygdala schizophrenia.

Methods are described for providing an objective assessment, risk prediction, and targeted therapeutics for patients with mood disorders via the use and analysis of specific blood biomarker panels in combination with traditional subjective mental health evaluations.

The disclosed methods can be used in the assessment, risk prediction, and targeted or individualized treatment of developed mood disorders. The methods can also be useful in preventive approaches, before a full-blown mood disorder manifests itself or re-occurs. Prevention may be further affected with social, psychological, or biological interventions (i.e. early targeted use of medications or nutraceuticals). The disclosed methods can be used to either supplement, or replace, existing or later-developed social, psychological, or biological interventions. Given the fact that 1 in 4 people will have a clinical mood disorder episode in their lifetime, that mood disorders can severely impact a person's quality of life, and that not all patients respond to current treatments, the need for, and importance of, the disclosed methods and related subject matter cannot be overstated.

Decades of work in mental health have shed light on possible molecular underpinnings of mood disorders. However, as the brain cannot be readily biopsied in live individuals, it is essential to be able to identify and validate accessible biomarkers for subsequent practical implementation in clinical settings. Blood gene expression profiling was utilized to identify genes and peripheral biomarkers predictive of brain and mood disorders that previously, in clinical practice, relied on purely subjective components and/or assessments to identify. Whereas the ascertainment of mood can be attempted with a clinical interview, the reliance solely on subjective patient self-report(s) to assess the severity and/or veracity of a mood disorder are a fundamental problem. The disclosed blood biomarkers that are predictive of mood disorders therefore provide a critical objective measurement to inform clinical assessments and treatment decisions.

Recent work has identified potential blood gene expression biomarkers for mood state using a case-case design and a visual analog-scale (VAS) (Le-Niculescu, H., et al. Identifying blood biomarkers for mood disorders using convergent functional genomics. Mol Psychiatry 14, 156-174 (2009)), validated independently as tracking response to cognitive-behavioral therapy by another group (Keri et al. Journal of Affective Disorder 2014). VAS avoids the issue of corrections for multiple comparisons that would arise if one were to look in a discovery fashion at multiple phenes in a comprehensive phenotypic battery (PhenoChipping) changed in relationship with all genes on a GeneChip microarray, which would require larger sample cohorts.

Patients having or potentially having psychiatric disorders may have an increased vulnerability to mood disorders, regardless of their primary diagnosis, as well as increased reasons for mood disorders, due to the often-adverse life trajectories suffered by these patients. As such, such patients can form a particularly suitable population in which to identify blood biomarkers for mood disorders that are generalizable and transdiagnostic.

This disclosure includes extensive blood biomarker gene expression studies performed in both male and female subjects diagnosed with major psychiatric disorders. In general, these populations of subjects having major psychiatric disorders exhibit increased incidence of co-morbidity with mood disorders and mood variability than do matched populations of subjects not diagnosed with major psychiatric disorders. These comorbidities in these populations suggest potential molecular-level co-morbidities between at least some major psychiatric disorders and mood disorders. Further evidence for a linkage between a major psychiatric disorder and a mood disorder is indirectly supported in part by the fact that some medications used to treat mood disorders (e.g., antidepressants, mood stabilizers) may be repurposed to treat major psychiatric disorders such as post-traumatic stress disorder (PTSD) and schizoaffective disorders. Still more evidence for a link between these morbidities comes from the fact that some therapeutic antipsychotics may be repurposed to treat some mood disorders. In the spirit of Research Domain Criteria (RDoC), this disclosure includes methods that intergrade multiple levels of information, from behavior and self-reports to gene expression, to enable optimized and specific psychiatric diagnosis and treatment. Many of the therapeutics disclosed herein are repurposed from their originally approved indications to treat one or more mood disorders based on the data obtained herein.

The exemplary studies and data performed herein were performed in a comprehensive fashion and provide a systematic approach to understanding and using precision medicine to enhance clinical diagnosis, treatment, and monitoring of mood disorders. Precision medicine represents a developing approach for disease treatment and prevention by taking into account an individual's variability in genes, environment, and lifestyle.

Convergent Functional Genomics (CFG) is an approach for identifying and prioritizing candidate genes and biomarkers for complex psychiatric and medical disorders by integrating and tabulating multiple lines of evidence: gene expression and genetic data, from human studies and animal model work. In a GFG analysis, the prioritization score of a gene or biomarker increases as the number of times it is correlated with a given condition or disorder increases. In the instant GFG analysis, the more often a gene or biomarker correlates with a given mood disorder the higher the likelihood that the gene or biomarker is associated with a given mood disorder. GFG may be characterized as a ‘fit-to-disease approach’, that extracts and prioritizes in a Bayesian fashion the connection between one or more biologically relevant signal(s) and a given disorder. The GFG approach is especially powerful in that it can make use of studies carried out on the same numbers of subjects.

Disclosed herein are methods for improved clinical diagnosis, treatment, and monitoring of mood disorders through precision medicine.

In various aspects, the mood disorder can be selected from a group of mood disorders consisting of depression; a bipolar disorder; an anxiety disorder; a condition characterized by an atypical mood, wherein the atypical mood is selected from stress, hormonal mood swings, Mild Cognitive Impairment, a substance-induced mood disorder, dementia, Alzheimer's disease, Parkinson's disease, Huntington's disease, and a psychotic disorder; or combinations of any of the foregoing. The mood disorder can be a Major Depressive Disorder (MDD). A major depressive episode is characterized by the presence of a severely depressed mood that generally persists for at least two weeks. A major depressive episode may be an isolated episode or recurrent; the episodes are categorized as mild (e.g., few symptoms in excess of minimum criteria), moderate, or severe (e.g., marked impact on social or occupational functioning). In other embodiments, the mood disorder is a bipolar disorder. In the past, if a patient has had an episode of mania or markedly elevated mood, typically a diagnosis of bipolar disorder is made. In some cases, the bipolar disorder can arise from a depressed or mixed phase of bipolar disorder.

The disclosed methods provide for an improved clinical diagnosis, improved treatment arising from the improved categorization and diagnosis, and optionally improvements in the monitoring of a subject diagnosed with a mood disorder. This approach can result in significantly better outcomes including fewer side effects and/or negative sequelae. For example, the therapeutics described herein can be administered at doses that effectively treats a subject with a mood disorder at a dose that reduces the risk that the subject will suffer adverse side effects. Common side effects may arise with any prescription mood disorder treatments, even those that are utilized in a manner consistent with their approved labelling instructions. As the repurposed therapeutics for use in the disclosed methods are readily commercially available, some of which are even sold over-the-counter without a prescription, such repurposed but patient-specific therapeutics have the benefit of being widely accessible and generally recognized as safe (GRAS) by one or more regulatory authorities, as compared to prescription anti-depressants and mood stabilizers.

### Mood Disorders

Provided herein are methods for using a blood test (or the use of other biological samples) for assessing a patient having a mood disorder (using a panel of 23 blood biomarkers), depression (using panel of 12 blood biomarkers), a bipolar disorder (using a panel of six blood biomarkers) or mania (using a panel of two blood biomarkers) to generate a patient-specific score, percentile ranking, and a traffic-light-type risk call for the identified mood disorders, depression, bipolar disorder, and/or mania for the subject.

The blood test measures the expression level of the panel of blood biomarkers and generates an expression score for each individual biomarker (also known herein as BioM). Other biological samples can be used in addition to blood. These biomarkers can also be assessed from saliva, urine, serum, or fat tissue biopsy.

To generate a patient-specific mood disorder score, each biomarker has a weighted value (known herein as CFE score or CFE Polyevidence Score). The expression score (BioM) is multiplied by the weighted value (CFE score) of the biomarkers to generate a weighted biomarker score, i.e., [BioM]×[CFE score]=weighted biomarker score. The weighted biomarker scores are added together for all biomarkers in a given panel to generate a score (mood disorder score, depression score, bipolar disorder score, or mania score), as represented by the following equation:

Score=Σ(BioM×CFE).

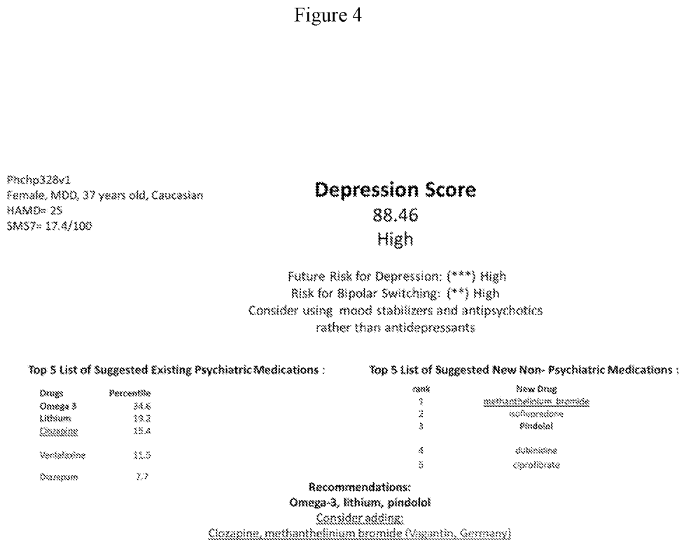

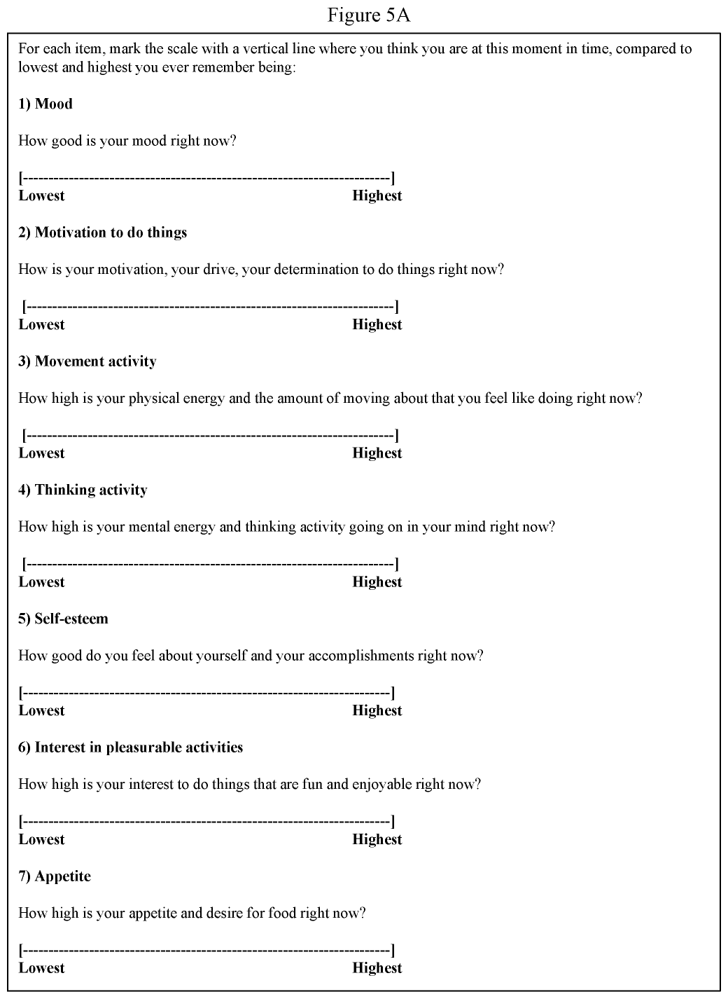

The percentile ranking may be generated for a subject by comparing the particular score determined for a subject by analyzing a sample from the subject for the presence of one or more biomarkers that correlated with a mood disorder, depression, bipolar disorder, or a mania of a subject with the average score of subjects whose clinical outcomes are known and are compiled in a database. A risk call, such as a traffic-light-type risk call using the colors green, yellow and red, can be generated based on the comparison of the score with patients in a database of clinical research studies. Green (also known herein as “Low Risk”) is given if the score on a new patient is below the average of the low risk research subjects tested in the past. Yellow (also known herein as “Intermediate Risk”) is given if the score is between the average of the low-risk subjects and average of the high-risk subjects. Red (also known herein as “High Risk”) is given if the score is above the average of the high-risk subjects. The risk call can also be categorized numerically or even in a binary fashion, e.g., risk/no risk. The risk score plus the rating can be provided in a report, see, e.g., FIG. 4, which illustrates a personalized patient report. Such a report can be generated for clinician use and phrased in a fashion for use by a medical professional (e.g., reflecting the objective assessment of depression state, future risk of severe depression, risk of bipolarity, matching with existing psychiatric medications, matching with non-psychiatric/repurposed medications, and monitoring response to treatment). A risk call can also utilize indicators other than color indicators, such as +1, 0, −1 or simply a binary system.

The biomarkers can be weighted using a CFE score, calculated using evidence from the different steps of the procedures to identify biomarkers:
i. assigning points for evidence from the discovery step;
ii. assigning points based upon CFG prioritization;
iii. assigning points based on validation; and
iv. assigning points based on testing in independent cohorts, with testing for at least one of the following traits: state low mood state, clinical depression, trait first-year hospitalization with depression, trait all future hospitalizations with depression, high mood state, clinical mania, first-year hospitalization with mania, all future hospitalizations with mania—with more points given if a given biomarker proves to be significantly predictive in all test subjects, less points if significantly predicts by gender only, and fewer points if significantly predicts by gender/diagnosis only.

Using this system, the total score for each biomarker as assayed in a subject can range, in an embodiment from zero to 48 points: 36 points from the data as calculated herein and 12 from literature data using CFG. In accordance with the methods disclosed herein, the empirical data obtained was weighted three times as much as the literature data, as it is functionally related to mood as measured using 3 independent cohorts (i.e., a discovery, a validation, and a testing cohort).

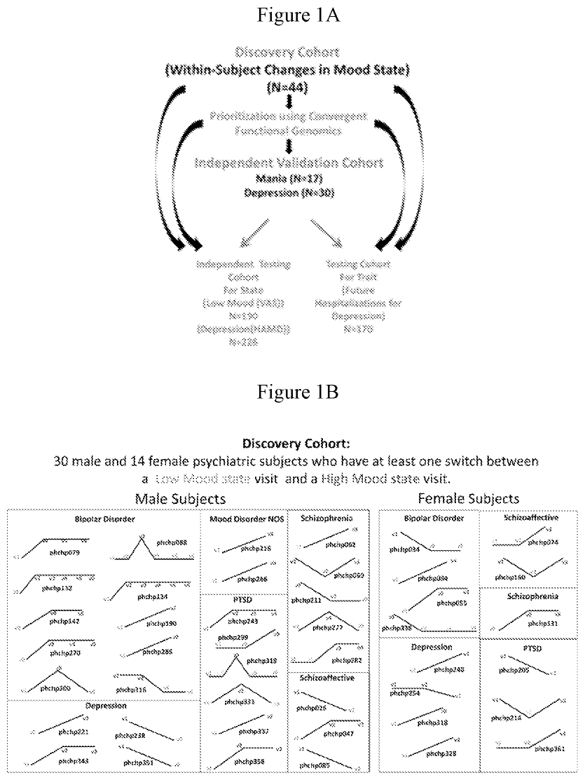

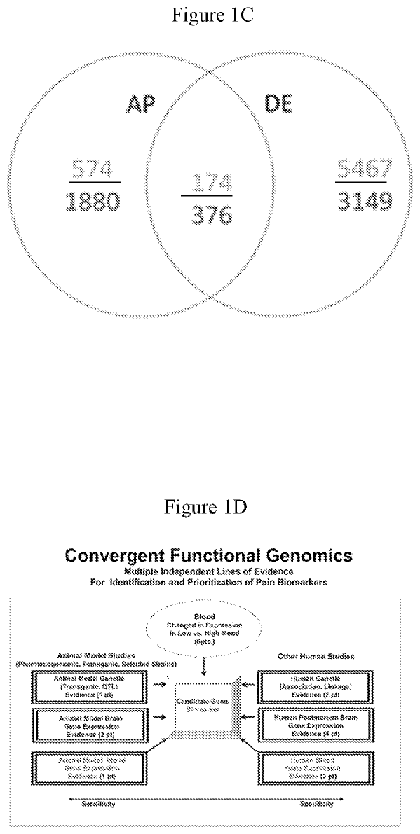

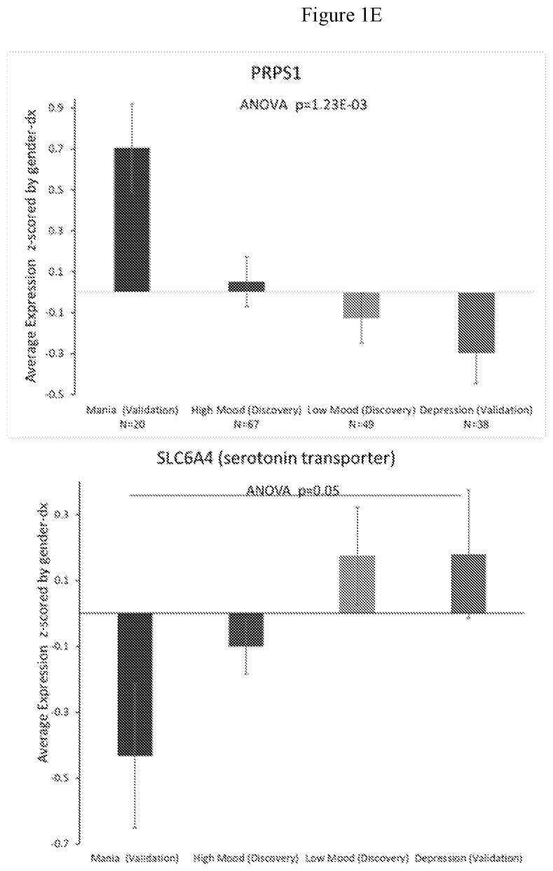

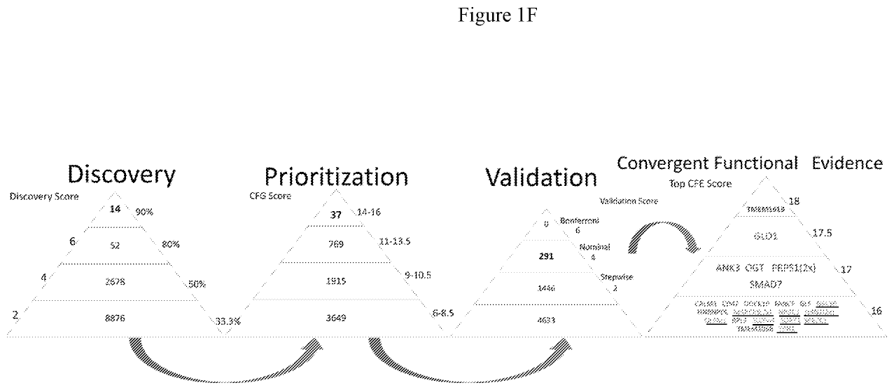

In some cases, the CFE score can be used for each given biomarker as depicted in FIG. 1F. In certain instances, the obtained CFE score depicted in the tables is within a range of a score; the obtained values can have an error rate of up to of +/−5 points.

The expression score obtained for each individual biomarker can be determined by either a cross-sectional method (when only one blood sample is available for a given patient) or via a longitudinal method (when multiple blood test samples from multiple patient visits are available). Raw gene expression data for each blood biomarker in a blood sample can be normalized (e.g., first by RMA normalization for technical variability, next by gender and then by diagnosis for biological variability) thereby obtaining an expression score. If a biomarker's expression level is increased in the disease state, it will have a positive sign before it. If the biomarker's expression level is decreased in the disease state, it will have a negative sign in front of it.

A panel of 22 blood biomarkers for assessing mood disorders in a subject can include NRG1, TMEM161B, PRPS1, GLS, DOCK10, GLO1, HNRNPDL, FANCF, SMAD7, CD47, OLFM1, CALM1, SPECC1, ANK3, OGT, RPL3, TPH1, MARCKS, TMEM106B, SORT1, GSK3B, and NR3C1 (Table 5).

A panel of 12 blood biomarkers for assessing, tracking and predicting depression in a subject can include NRG1, DOCK10, GLS, PRPS1, TMEM161B, GLO1, FANCF, HNRNPDL, CD47, OLFM1, SMAD7, and SLC6A4 (Table 3A).

A panel of six biomarkers for assessing, tracking and predicting both depression and mania, hence bipolar mood disorders, can include NRG1, DOCK10, GLS, PRPS1, TMEM161B, and SLC6A4 (Table 3B). A panel of two biomarkers for assessing, tracking and predicting mania can include RPL3 and SLC6A4 (Table 3C).

When a blood test is used to generate a patient-specific profile of blood biomarkers for a mood disorder to match a patient with existing drugs used in clinical care, the expression score for each biomarker can be determined as described herein. Then, each biomarker's expression score can be compared with a reference subject expression level for that biomarker. A “reference expression level” can be the average value of the expression level of the biomarker in high-risk research subjects tested in previous clinical research studies or can be the expression level of the biomarker at a previous testing time-point in the same patient. Using a “reference expression level” for comparison can assist to determine the percentage of patients with a comparable expression level of the biomarkers, and for whom the expression level and/or mood disorder was modulated by treatment with an existing clinical care drug. This comparison can be used to rank each drug as a potential match for treatment of a mood disorder in the patient.

When a blood test is used to provide a patient-specific gene blood marker signature for matching with one or more repurposed therapeutic agents, the expression score for each blood gene biomarker can be determined as described herein. Each blood gene biomarker in the panel can be designated as “increased” (I) when the expression level of the biomarker is higher than the expression level of same biomarker determined in a matched reference population of patients diagnosed as not suffering from a particular mood disorder. Similarly, each blood gene biomarker can be designated as “decreased” (D) when the expression level of the biomarker is lower than the expression level of same biomarker determined in a matched reference population of patients diagnosed as not suffering from a particular mood disorder. The panel of biomarkers containing this designation (I or D) for each biomarker can then be compared with a drug database to identify drugs that effect the expression of these gene biomarkers. This type of analysis may be used to identify drugs that may be repurposed as therapeutic agents for the treatment of mood disorders. As detailed herein, the drug database may be the Connectivity Map, the NIH's Library of Integrated Network-Based Cellular Signatures (LINCS), or equivalent or similar databases that use a network-based matching system to identify therapeutic agents that may act to decrease the expression of increased biomarkers or increase the expression of decreased markers in a subject having an expression profile identified as diagnostic for certain mood disorders. In some examples, the matching of blood biomarker signatures determined for a subject and the biomarkers in a matched references population can be done in a gender-specific manner. Matching may be done to existing psychiatric medications based on individual biomarkers that are changed in expression upon treatment with the medication, and ranking those medications based on which of them has the greatest impact the most biomarkers. Signatures means the group/panel of biomarkers changed in an individual, that can be used to match with existing psychiatric drugs, or to new method of use, non-psychiatric, repurposed drugs.

The CFE score for each biomarker can be used as depicted in FIG. 1F. The CFE score depicted in these tables can lie within a range of score values.

The expression score for each individual biomarker can be determined by either a cross-sectional method (e.g., when only one blood sample is available for a given patient) or via a longitudinal method (e.g., when multiple blood test samples from multiple patient visits are available). As further detailed below and in the Examples, according to these methods, the raw gene expression data for each blood biomarker gene in the blood sample can be normalized (e.g., first by RMA normalization for technical variability, next by gender and diagnosis for biological variability) and providing a normalized expression score. If a blood biomarker's expression level is increased in the subject, the expression can be denoted by a positive sign before it (e.g., +1). If a blood biomarker's expression level is decreased in the subject, it can be denoted by a negative sign (e.g., −1).

The described methods can further or optionally comprise the step of monitoring the effectiveness of a treatment in a subject.

A disclosed method can be designed to be used with ease at a point-of-care facility. Additionally, a disclosed method may be conducted in part or whole in clinical laboratory settings, hospitals, clinics, doctor's offices, other points of psychological or psychiatric care, research labs, and/or any laboratory-based testing environment where cellular or molecular-biological testing can be performed.

The disclosed methods utilize a blood biomarker gene database. A database can include data for more than one blood biomarker related to mood disorders. A tested patient can be normalized against a database, which contains blood biomarker data from similar patients already tested for one or more mood disorders, and optionally further compared to the database for ranking and risk prediction purposes. As patient databases increase their data, normative population levels of each blood biomarker and/or panel of blood biomarkers may be further established, similar to other laboratory measures. Such blood biomarker databases having normative blood biomarker levels may be accessed and used regardless of the diagnostic platform used to identify the blood biomarker and/or blood biomarker expression level. For example, blood biomarkers may be detected by analyzing the expression level of RNA transcripts, protein, peptides, or fragments thereof. In some aspects, biomarkers may be detected and/or measured using microarray gene expression, RNA sequencing, polymerase chain reaction (PCR), real-time PCR (rtPCR), quantitative PCR (qPCR), immunohistochemistry, enzyme-linked immunosorbent assays (ELISA), and antibody arrays, and the like.

The methods disclosed herein can utilize one or more blood biomarker panels that are predictive of one or more mood states. For example, a panel of universal mood disorder blood biomarkers (i.e., biomarkers that are predictive in all mood disorders) can be used in combination with a panel of personalized mood disorder blood biomarkers, for example that are predictive by gender and/or diagnosis. The methods can utilize a panel of universal mood disorder blood biomarkers. Alternatively, the method can utilize a panel of personalized mood disorder blood biomarkers. The type of personalized biomarker panel used in a method disclosed herein can vary. For example, the personalized biomarker panel can be selected from a male mood disorder blood biomarker panel, a female mood disorder blood biomarker panel, a male depression blood biomarker panel, a female depression blood biomarker panel, a male bipolar blood biomarker panel, a female bipolar blood biomarker panel, a male mania blood biomarker panel, a female mania blood biomarker panel, a depression blood biomarker panel, a bipolar blood biomarker panel, a mania blood biomarker panel, or combinations of such biomarkers (blood or from other biological sample) thereof.

A panel of 23 biomarkers can be sufficient to assess, diagnose, treat and/or monitor one or more mood disorders in a subject in need thereof. A panel of 2, 6, or 12 biomarkers can be sufficient to assess, diagnose, treat and/or monitor one or more mood disorders in a subject. A panel of six biomarkers can be used to assess a bipolar disorder in a subject. A panel of 2 biomarkers can be used to assess a mania. Larger or smaller biomarker panels can also be utilized, for example biomarker panels can comprise 1, 2, 3, 4, 5, 6, 7, 8, 9, 10, 11, 12, 15, 17, 20, 22, 25, 27, 30, 25, 40, 45, 50, 55, 60, 65, 70, 75, 80, 85, 90, 95, 100 (as well as any integer between the listed values) or more biomarkers and can be used with the disclosed methods.

Variable quantitative scoring schema can be designed using, for example, the algorithm used herein. Such algorithm may include a variable selection, or a subset feature selection algorithm may be used. Both statistical and machine learning algorithms are suitable for devising a framework to identify, rank, and analyze association between marker data and phenotypic data (e.g., mood disorders). An analysis of a plurality of blood biomarkers, for example, a panel of about 23, 16, six, or two blood biomarkers, may be carried out separately or simultaneously within one test sample. For example, several blood biomarkers may be combined into one test for efficient processing of multiple samples. In some aspects there may be value in testing multiple samples (for example, at successive time points) from the same individual. Such testing of serial samples may allow the identification of changes in blood biomarker levels over time, within a period of interest, or in response to a certain treatment.

A particular panel of blood biomarkers may represent the preferred universal biomarkers for assessing and diagnosing mood (i.e., biomarkers that are predictive in all populations). A panel of blood biomarkers may instead represent personalized biomarkers (i.e., biomarkers that are predictive individually, by gender and/or by diagnosis), such that one or both of the panels may be used to assess, diagnose, treat and/or monitor one or more mood disorders. In a clinical setting, a blood sample from a subject might be tested for expression levels of more than one panel of any of the blood biomarkers described herein.

In clinical practice, it may be advantageous for every new patient who is tested to be normalized against a database of similar patients already tested, and compared to them for ranking and risk prediction purposes, regardless if a platform like microarrays, ribonucleic acid (RNA) sequencing, or a more targeted one like PCR is used in the end clinically. As databases grow larger, normative population levels can and should be established, similar to any other laboratory measures.

### Blood Biomarkers

Biomarkers are molecules, proteins, cells, hormones, enzymes, genes, or gene products that can be detected and measured in parts of the body like blood, saliva, urine, or tissue. Biomarkers may indicate normal or diseased states, for example by being upregulated in response to, or because of, a specific disease state and thus present in higher than normal levels, or vice versa; because of this, biomarkers are emerging as important tools in the detection and diagnosis of diseases that are traditionally characterized by unreliable subjective diagnosis methods, such as self-reporting. Blood biomarkers are useful for detection and diagnostic methods due to the relative ease of obtaining blood samples from a subject. Other biological samples may also be used to measure biomarkers, including but not limited to saliva, cerebrospinal fluid (CSF), serum, urine, stool, aspirates, and/or another bodily fluid. Biomarkers may also be detected and measured in a peripheral tissue sample.

The amount of a blood biomarker used in the disclosed methods indicates the presence or absence of a disease state (i.e., a mood disorder). As used in this context, the “amount” of a blood biomarker can mean the presence or absence of the biomarker in a blood sample, or an indication of the biomarker expression level, any one of which may be used to associate or correlate a phenotypic state (i.e., the presence or absence of a mood disorder). The biomarker expression level indication can be direct or indirect and measure over- or under-expression, or the presence or absence, of a biomarker given the physiologic parameters and in comparison, to an internal control, normal tissue or another phenotype. Nucleic acids or proteins or polypeptides or portions thereof used as markers are contemplated to include any fragments thereof, in particular, fragments that can specifically hybridize with their intended targets under stringent conditions and immunologically detectable fragments. One or more biomarkers may be related. A biomarker may also refer to a gene or DNA sequence having a known location on a chromosome and associated with a particular gene or trait. Genetic markers associated with certain diseases or for pre-disposing disease states can be detected in the blood and used to determine whether an individual is at risk for developing a disease. Levels of biomarker gene expression and protein levels are quantifiable and the variation in quantification or the mere presence or absence of the expression may also serve as biomarkers. Using proteins/peptides as biomarkers can include any method known in the art including, without limitation, measuring amount, activity, modifications such as glycosylation, phosphorylation, ADP-ribosylation, ubiquitination, immunohistochemistry (IHC), and the like.

A panel of blood biomarkers for assessing and/or the diagnosis, treatment, and monitoring of mood disorders can include one or more of the group of gene biomarkers consisting of: TMEM161B, GLO1, PRPS1, SMAD7, ANK3, OGT, NRG1, GLS, DOCK10, HNRNPDL, FANCF, CD47, RPL3, OLFM1, CALM1, TPH1, SPECC1, MARCKS, TMEM106B, SORT1, GSK3B, NR3C1, and SLC6A4.

A panel of blood biomarkers for assessing and/or the diagnosing, treating, and/or monitoring of depression can include one or more of the biomarker genes from the group consisting of: NRG1, DOCK10, GLS, PRPS1, TMEM161B, GLO1, FANCF, HNRNPDL, CD47, OLFM1, SMAD7, and SLC6A4.

A panel of blood biomarkers for assessing and/or the diagnosing, treating, and/or monitoring a bipolar mood disorder can include one or more of the biomarker genes from the group consisting of: TTLL3, CREBBP, DRD3, CKB, TRPM6, and MORF4L2

A panel of blood biomarkers for assessing and/or the diagnosing, treating, and/or monitoring of a mania can include one or more of the biomarker genes from the group consisting of: RPL3 and SLC6A4.

### Existing Drugs in Clinical Care for Mood Disorders

There are a number of medications available and used for the clinical treatment of mood disorders, but they vary widely in their side effects and are not equally effective across all patient populations. As such, the current standard of clinical treatment of mood disorders is notoriously imprecise and in need of a more individualized approach to therapeutic administration. Methods provided by the present disclosure, using the specific blood tests detailed herein, can generate a patient-specific profile that is used for matching the patient with existing drugs used in clinical depression or bipolar care, to identify the known therapeutics that are the most efficacious for the specific patient, on the basis of their biomarker expression levels. In doing so, the therapeutic identified for administration may be one previously developed and specifically approved to treat depression or bipolar disorder, however, and may not be regularly associated with the treatment of that particular mood disorder.

As disclosed herein, the use of panels of blood biomarkers in a patient can be used to identify the optimal therapeutics specific to that patient, for the treatment of depression or bipolar disorder or other mood disorder. A therapeutic can be a drug or drug combination clinically used in the treatment of mood disorders. Blood biomarkers can be used for measuring the patient's response to a given treatment via pharmacogenomics (the study of how genes affect a person's response to drugs. Alternatively, when biomarkers connected with multiple different drug/classes are changed in an individual, the disclosed methods can create a prioritization of drugs, based on the change in the proportion or percentile of biomarkers. This may enable the optimization of a drug or combination of drugs, via targeted rational polypharmacy, based on the biomarker panel expression changes.

A patient's blood biomarker expression levels also can be used in combination with and/or in comparison to normalized scores from other patients to enable drug discovery and repurposing for mood disorders, such as depression or bipolar disorder. For example, the higher the proportion/percentile of over- or under-expressed biomarkers present for a certain drug/class, the more likely that a drug or a therapy from that drug/class would be efficacious in treating the particular patient indicating as having a particular disease or disorder. Sometimes, a therapeutic may be broadly applicable across a mood disorder diagnosis.

### Repurposed Therapeutic Agents

Methods provided by the present disclosure can be utilized to identify one or more repurposed therapeutics that will be useful to treat an individual experiencing a mood disorder. Such therapeutics are being repurposed for the treatment of mood disorders using disclosed methods.

Drug repurposing refers to a strategy by which a new value is generated from a drug or other therapeutic by targeting a disease other than those diseases for which the drug or other therapeutic was originally intended or approved. There are several advantages associated with using repurposed drugs in the treatment of mood disorders; for example, such drugs can have toxicology and pharmacology profiles with fewer side effects while providing increased effectiveness. As used herein, “therapeutic agent,” “therapy,” “drug” and/or “repurposed drug” refers to any agent or compound useful in the treatment, prevention, or inhibition of a mood disorder or mood-related disorder, as identified by the disclosed methods.

As disclosed herein, the measurement of blood biomarkers in a patient can be used to identify therapeutic agents specific to that patient, for the treatment of a mood disorder. The blood test can also be used to provide a patient-specific signature that is compared with a drug database to identify repurposed therapeutic agents for the treatment of the patient's signature of depression, given the patient's biomarker expression. The therapeutic can be one or more repurposed drugs. Blood biomarkers can be used for measuring the patient's response to the treatment via pharmacogenomics. Additionally, when biomarkers for multiple different drug/classes are changed for a patient, the disclosed methods can create a prioritization based on the proportion/percentile of biomarkers for each class to choose the optimal drug or combination of drugs, via targeted rational polypharmacy.

A patient's blood biomarker expression level can be used in combination with and/or in comparison to that from other patients to enable drug discovery and repurposing for mood disorders. For example, the higher the proportion/percentile of over- or under-expressed biomarkers present for a certain drug/class, the more likely that drug or therapy would be for treatment.

A therapeutic agent can be broadly applicable across a mood disorder diagnosis. Sometimes, therapeutic agents may be more narrowly applicable for subjects with a specific mood disorder diagnosis. In some examples, isoflupredone, trichostatin A, dubinidine, ciprofibrate, pioglitazone, tropine, adiphenine, saquinavir, amitriptyline, and/or chlorogenic acid may be used as a therapeutic for the treatment of a mood disorder. In some examples, pindolol, ciprofibrate, pioglitazone adiphenine, asiaticoside, chlorogenic acid, or combinations thereof may be the therapeutic for the treatment of depression. In other examples, pindolol, lansoprazole, xamoterol, methanthelinium bromide, asiaticoside, estradiol, methacholine, isoflupredone, carteolol, chlorcyclizine or combinations thereof may be used for the treatment of depression. In other examples, valproic acid, atracurium besylate, Chicago Sky Blue 6B, enoxacin, levobunolol, 15-delta prostaglandin, ciprofibrate J2, pirinixic acid, isoflupredone, trichostatin A or combinations thereof may be used the drug for the treatment of depression and bipolar disorder. A targeted therapeutic as identified using the disclosed methods can be specific to a mood disorder diagnosis and/or specific to a gender.

Tables 4A1-4B1 denote examples of targeted therapeutics for drug repurposing for depression. Drugs that have opposite gene expression effects to the gene expression signature of our nominally significant predictive biomarkers for depression (Tables 4A1-4A2) and for bipolar depression (Table 4A3), using the Connectivity Map (see Lamb, J., et al. The Connectivity Map: using gene-expression signatures to connect small molecules, genes, and disease, Science 313, 1929-1935 (2006)) (CMAP), and for depression (B1) using the NIH LINCS database (Table 4B1). Bold font indicates new drugs of immediate interest. Italicized font indicates a natural compound. Underlined font indicates known drugs that serve as a de facto positive control. Table 4A1 depicts “Drugs Identified Using Gene Expression Panels of Biomarkers with Highest Evidence (CFE) for involvement in Depression” (BioM12 Depression—12 genes, 13 probe sets). Direction of expression in high mood. (Out of 13 probe sets, 8 increased and 3 decreased probe sets were present in HG-U133A array used by CMAP). Table 4A2 depicts Drugs Identified Using Gene Expression Panels of Biomarkers with Highest Evidence (CFE) for involvement in Depression without overlap with bipolar (BioM6 Depression Specific—6 genes, 7 probe sets). Direction of expression in high mood. (Out of 7 probe sets, 5 increased and two decreased biomarkers were present in HG-U133A array used by CMAP). Table 4A3 depicts “Drugs Identified Using Gene Expression Panels of Biomarkers Overlapping between Depression and Bipolar” (BioM6 Bipolar—6 genes, 6 probe sets). Direction of expression in high mood. (Out of 6 probe sets, 4 increased and 1 decreased probe sets were present in the HG-U133A array used by CMAP). Table 4B1 depicts Drugs Identified Using Gene Expression Panels of Biomarkers with Highest Evidence (CFE) for involvement in Depression (BioM12 Depression—12 genes)). Direction of expression in high mood (9 increased and 4 decreased).

### Computer Implemented Methods

A disclosed method can include or optionally include computer implemented methods for analysis of specific blood biomarker panels in combination with traditional subjective mental health evaluations to provide enhanced assessment, risk prediction, and targeted therapeutics, and monitoring for patients with mood disorders. An exemplary method can include the steps of (a) storing a database of biological data for a plurality of patients, the biological data that is being stored including for each of said plurality of patients (i) a treatment type, (ii) at least one blood biomarker or panel of blood biomarkers associated with a mood disorder, and (iii) at least one disease progression measure for the mood disorder from which treatment efficacy can be determined; and then (b) querying the database to determine the dependence on the marker of the effectiveness of a treatment type in treating the mood disorder, to thereby identify a proposed treatment as an effective treatment for a subject carrying the marker correlated with the mood disorder.

Blood biomarker information can be provided, via a network, to at least one database that stores the information. The blood biomarker information can be provided to the network using one or more wired links, one or more wireless links, and/or any suitable combination thereof. The network can be a wide area network, a local area network, and/or any other suitable type of network. The method can use a database that is a single database or can be comprised of multiple databases, or a serial combination of databases used over time. The method can use database(s) information that is stored in one or more publicly accessible databases.

The database(s) can store clinical mental health evaluation information, patient medical history information, blood biomarker expression data, and/or any other suitable information about the patient in any suitable format and/or using any suitable data structure(s).

The patient information and/or the publicly available information contained in the database(s) may be used to perform any of the methods described herein related to determining a score and/or therapy for a given patient. For example, the information stored in the database(s) may be accessed, via network, by software executing on server(s) to perform any one or more of the methods described herein. Exemplary methods can include determining a score and/or therapy based on one or more normalized biomarker scores. In some aspects, these methods include determining a score and/or therapy based on a panel of normalized biomarker scores.

Exemplary methods can utilize a software program that provides a visual representation of information related to a patient's individual blood biomarker scores, panel of blood biomarker scores, risk percentile, recommended therapy, and/or predicted efficacy of a given therapy and any combination thereof. For example, this information may be related to a patient's individual blood biomarker scores, panel of blood biomarker scores, individual normalized blood biomarker scores, and/or panel of normalized blood biomarker scores. Such a software program can execute in any suitable computing environment including but not limited to a device co-located with a user, one or more devices remote from the user, or a cloud-computing environment. This visual representation is provided/output in a written report on a screen, an e-mail, a graphical user interface, and/or any other suitable to be provided to one or more user(s). Such users can include, but are not limited to a patient, a doctor, a caretaker of a patient, a healthcare provider such as a nurse, or a person involved with a clinical trial.

A number of biomarkers identified herein and as disclosed in the panels have biological roles that are related to the circadian rhythm (clock) (Table 7). From the literature, a database of all the known circadian rhythm-related genes was compiled (numbering a total of 1,468 genes). The compiled list of circadian rhythm-related genes was used to ascertain all the genes in the dataset that were circadian and provide estimates of enrichment of circadian genes in the identified biomarkers. Out of the 23 mood disorder biomarker genes identified, eight biomarker genes had circadian rhythm evidence (35%). The indication that 35% of the 23 disorder biomarker genes had circadian rhythm evidence suggests a 5-fold enrichment for circadian genes over genes having other function. This enrichment for genes having circadian rhythm function suggests that there may be a molecular underpinning for the epidemiological data between disrupted sleep and mood disorders, and for the clinical phenomenology of seasonal components to mood disorders. As disclosed herein, the mood disorder biomarkers also had prior evidence of involvement in other psychiatric and related disorders (Table 7), providing a molecular basis for co-morbidity, and the possible precursor effects of some of these disorders on mood, and conversely, the precursor role of mood in some of them.

As detailed herein, a comprehensive approach was undertaken to identify blood biomarkers for mood disorders and to identify repurposed specific therapeutic agents related to the expression of these blood biomarkers. This approach identified definitive biomarkers for mood disorders in general, and depression in particular, with a focus on biomarkers that are transdiagnostic, by studying mood in psychiatric disorders patients (e.g., depression and bipolar disorder, as well as schizophrenia, schizoaffective disorder, and PTSD). Disclosed herein is this systematic discovery, prioritization, validation, and testing approach.

For the discovery steps, a hard to accomplish but powerful within-subject design was used, with an N of 44 subjects with 134 visits. A “within-subject study design” factors out genetic variability, as well as some medications, lifestyle, and demographic effects on gene expression, permitting identification of relevant signal with Ns as small as 1 (see Chen, R., et al., “Personal omics profiling reveals dynamic molecular and medical phenotypes,” Cell 148: 1293-1307 (2012)). A further benefit of a within-subject design is that it standardizes for accuracy/consistency of self-report of psychiatric symptoms (“phene expression”), similar in rationale to the signal detection benefits it provides in gene expression.

First, a “longitudinal within-subject design” and whole-genome gene expression approach were used to discover biomarkers that track mood state in subjects who had diametric changes in mood state from low to high, from visit to visit, as measured by a simple visual analog scale/app that had previously been developed (Simple Affective State Scale, SASS). One of the biomarkers decreased in expression in blood in high mood states was SLC6A4 (the serotonin transporter, the target of SSRIs), which was used as a de facto positive control that our approach ends up with biomarkers that are clinically relevant for mood disorders.

First, blood gene expression biomarkers for mood were determined using a longitudinal design, looking at differential expression of genes in the blood of male and female subjects with psychiatric disorders (e.g., bipolar disorder, major depressive disorder, schizophrenia/schizoaffective, and post-traumatic stress disorder (PTSD)) and high-risk populations prone to mood disorders who constitute an enriched pool in which to look for blood biomarkers. This powerful longitudinal within-subject design was used in individuals with psychiatric disorders to discover blood gene expression changes between self-reported low-mood and high-mood states, measured by a visual analog scale (VAS), called the Simplified Affective State Scale (SASS), which has seven items related to mood. This analysis compared low-mood states to high-mood states using a powerful within-subject design to generate a list of differentially expressed genes (see Niculescu, A. B., et al., “Understanding and predicting suicidality using a combined genomic and clinical risk assessment approach,” Mol. Psychiatry 20: 1266-1285 (2015); Levey, D. F., et al., “Towards understanding and predicting suicidality in women: biomarkers and clinical risk assessment,” Mol. Psychiatry 21: 768-785 (2016); Le-Niculescu, H., et al., “Discovery and validation of blood biomarkers for suicidality,” Mol. Psychiatry 18: 1249-1264 (2013); and Chen, R., et al., “Personal omics profiling reveals dynamic molecular and medical phenotypes,” Cell 148: 1293-1307 (2012)).

Next, a comprehensive Convergent Functional Genomics (CFG) approach was taken with a comprehensive database of knowledge in the field to date, to prioritize from the list of differentially expressed genes/biomarkers those that are of particular relevance to mood. CFG integrates multiple independent lines of evidence-genetic, gene expression, and protein data, from brain and periphery, from human and animal model studies, as a Bayesian strategy for identifying and prioritizing findings, reducing the false-positives and false-negatives inherent in each individual approach. In this approach, a list of candidate biomarkers was prioritized with a Bayesian-like Convergent Functional Genomics approach, comprehensively integrating previous human and animal model evidence in the field. Next, the mood disorder biomarkers from discovery and prioritization were themselves prioritized in an independent cohort of psychiatric subjects with clinically severe depression (which was and can be measured using the Hamilton Depression Scale, “HAMD”) and/or with a diagnosis of clinically severe mania (which was and can be measured using the Young Mania Rating Scale, “YMRS”). Adding the scores from the first three steps into an overall convergent functional evidence (CFE) score, yielded 23 top candidate biomarkers that had a CFE score as at least or greater than SLC6A4, which serves as a positive control and threshold for these studies. The 23 top candidate biomarkers identified are: TMEM161B, GLO1, PRPS1, SMAD7, ANK3, OGT, NRG1, GLS, DOCK10, HNRNPDL, FANCF, CD47, RPL3, OLFM1, CALM1, TPH1, SPECC1, MARCKS, TMEM106B, SORT1, GSK3B, NR3C1, and SLC6A4. As previously indicated, a larger proportion of the genes identified are involved in circadian rhythm mechanisms. The biological pathways and networks for the top candidate biomarkers were analyzed, showing that circadian, neurotrophic, and cell differentiation functions are involved, along with serotonergic and glutamatergic signaling, supporting a view of mood as reflecting activity and growth.

Fourth, independent cohorts of psychiatric patients were tested for the ability of each of these top candidate biomarkers to predict a state (e.g., mood (SASS), depression (HAMD), mania (YMRS)), and a trait (e.g., future hospitalizations for depression, future hospitalizations for mania). The analyses were conducted across all patients, as well as personalized by gender and diagnosis, showing increased accuracy with the personalized approach, particularly in women.

Utilizing the above four steps, 12 biomarkers were identified having strongest overall evidence for tracking and predicting depression; the 12 identified biomarkers are: NRG1, DOCK10, GLS, PRPS1, TMEM161B, GLO1, FANCF, HNRNPDL, CD47, OLFM1, SMAD7, and SLC6A4. The top six biomarkers of these 12 with the strongest overall evidence for tracking and predicting both depression and mania, including bipolar mood disorders, were NRG1, DOCK10, GLS, PRPS1, TMEM161B, and SLC6A4. The six biomarkers (i.e., NRG1, DOCK10, GLS, PRPS1, TMEM161B, and SLC6A4) overlap completely with the depression list of biomarkers. Two biomarkers with the strongest overall evidence for mania identified are: RPL3 and SLC6A4. SLC6A4 is also present in the depression biomarker list. On all 3 lists (i.e., depression, bipolar and mania), SLC6A4 was used as the biomarker cuttoff, wherein to be present in a list a biomarker must have a CFE score better or equal to the CFE score of SLC6A4. The data as disclosed herein provides support for the view that, while mood is a continuum from low to high mood, with some of the best predictive biomarkers for low mood/depression and high mood/mania being shared (with changes in opposite direction in depression vs. mania)]), certain of the identified biomarkers are stronger predictors for clinical depression while other biomarkers are more predictive for clinical mania. This result is scientifically supported by the different co-morbidites associated with those conditions.

Next, the markers thus discovered, prioritized, and validated from the first three steps were tested in corresponding independent cohorts of psychiatric subjects to see their ability to predict a low mood state, a clinical depression state, and a future hospitalization with depression, in another independent cohort of psychiatric subjects. The blood biomarkers in all subjects in the test cohort were tested, as well as in a more personalized fashion by gender and psychiatric diagnosis. Parallel analyses for high mood/mania were carried out.

Finally, bioinformatics analyses on the blood biomarkers thus discovered, prioritized and validated were used to identify new/repurposed drugs for mood disorder treatment. In this work, the blood biomarkers were assessed for evidence for involvement in other psychiatric and related disorders and their biological pathways and networks were analyzed. Biomarkers that are targets of existing mood disorder drugs were identified, for pharmacogenomic population stratification and measuring of response to treatment for depression. The biomarker gene expression signatures were also used to interrogate connectivity databases and novel drugs and natural compounds that can be repurposed for treating and preventing depression were identified. The evidence for the mood disorder, depression, and mania biomarkers being targets of existing psychiatric drugs was also examined. This allows pharmacogenomic targeted treatments, and the measuring of response to treatment.

As disclosed herein, longitudinal monitoring of changes in blood biomarkers within an individual, also measuring most recent slope of change, maximum levels attained, and maximum slope of change attained, is more informative than only measuring cross-sectional comparisons of levels within an individual with normative population levels. For the blood biomarkers identified herein, combining the blood biomarker values into a convergent functional evidence (CFE) score, brings to the fore blood biomarkers that have prioritized clinical utility for objective assessment and risk prediction for depression, mania, and bipolar mood disorders (Tables 3 and 5). These biomarkers may be utilized individually and/or in polygenic panels of biomarkers with CFE weights.

These and other benefits and aspects of this disclosure will be more appreciated in view of the data and test results described in the following examples and accompanying Figures.

### EXAMPLE 1

Step 1: Biomarker Discovery. Candidate blood gene expression biomarkers were identified, biomarkers which:
1. change in expression in blood between self-reported low-mood and high-mood states;
2. track the mood state across visits in a subject; and
3. track the mood state in multiple subjects.

A visual analog measure for mood state (SMS-7) was used. At a phenotypic level, the SMS-7 quantitates a mood state at a particular moment in time, and normalizes mood measurements in each subject, comparing the mood measurements to the lowest and highest mood measurements that a subject ever experienced. A powerful “within—subject and then across-subject design” was used in a longitudinally followed cohort of subjects who displayed at least a 50% change in the mood measure between different testing visits (n=44 subjects with 134 visits), to identify differentially expressed genes that track mood state. As previously described (Niculescu, A. B., et al., “Understanding and predicting suicidality using a combined genomic and clinical risk assessment approach,” Mol. Psychiatry 20: 1266-1285 (2015)), a 33% of maximum raw score threshold (internal score of pt.) was set, with 11,620 unique probe sets from Affymetrix, using both absent/Present (AP) analyses and Differential Expression (DE) analyses (FIG. 1D). The analyses were carried forward to the prioritization step (Example 2). The results represent approximately a 5-fold enrichment of the 54,625 probe sets on the Affymetrix array.

As depicted in FIG. 1A, the cohorts used in this study involved a flow of steps through discovery, prioritization, and validation of biomarkers for mood. FIG. 1B discovery cohort longitudinal within-subject analysis. Phchp ### is the study ID for each subject. V # denotes visit number. FIG. 1C depicts differential gene expression in the discovery cohort, the number of genes identified with differential expression (DE), and absent-present (AP) methods with an internal score of 2 and greater. No underlining indicates an increase in expression in High Mood; underlining indicates a decrease in expression in High Mood. At the discovery step, probe sets were identified based on their score for tracking Mood with a maximum of internal points of 6 (33% (pt.), 50% (pt.) and 80% (pt.)). FIG. 1D depicts prioritization with CFG for prior evidence of involvement in mood disorders. In the prioritization step, probe sets were converted to their associated genes using Affymetrix annotation and GeneCards. Genes were prioritized and scored using CFG for Mood evidence with a maximum of 12 external points. Genes scoring at least 6 points out of a maximum possible of 18 total internal and external scores points were carried to the validation step. FIG. 1E depicts blood biomarker validation in two independent cohorts of psychiatric patients with clinically severe depression (HAMD>=22) and clinically severe mania (YMRS>=20), showing that VTI1A and CCDC191 were the most significantly increased and decreased, respectively, biomarkers in low mood/depression. FIG. 1F depicts validation. In the validation step, biomarkers were assessed for stepwise change from the validation group with mania, to the discovery groups of subjects with high mood or low mood, to the validation group with depression, using ANOVA. N=number of testing visits. 291 biomarkers were determined to be nominally significant, and 1446 biomarkers were stepwise changed.

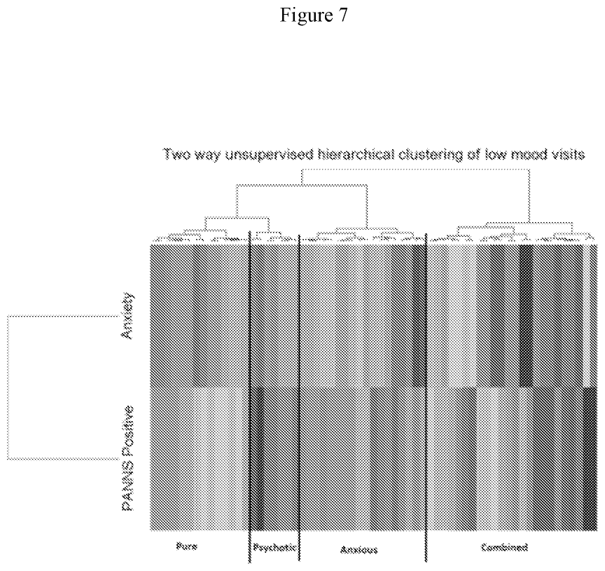

In the discovery cohort, it was investigated whether subtypes of low mood could be identified based on mental state at the time of low mood visits, using two-way hierarchical clustering with anxiety and psychosis measures. The mood state self-report of individual subjects may be more reliable in this cohort (i.e., the discovery cohort), as the subjects demonstrated the aptitude and willingness to report different, and diametric, mood states. Using this approach, four potential subtypes of low mood/depression were identified: high anxiety and low psychosis (anxious); high anxiety and high psychosis (combined); low anxiety and high psychosis (psychotic); and low anxiety and low psychosis (pure low mood) (FIG. 7). Patients diagnosed with low mood are patients that do not exhibit clinical symptoms of either high anxiety or high psychosis.

### EXAMPLE 2

Step 2: Prioritization. A Convergent Functional Genomics (CFG) approach was used to prioritize the candidate biomarkers identified in the discovery step (33% cutoff, internal score of >=2pt.) by using published literature evidence (e.g., genetic, gene expression and proteomic), from human and animal model studies, for biomarker involvement in mood disorders (FIG. 1D and Table 6). There were 6370 probe sets that had a total score (i.e., combined internal discovery score and external prioritization CFG score) of 6 and above. These 6370 probe sets were carried forward to be further analyzed in a validation step as further described in Example 3. The 6370 probe sets result represents approximately a 10-fold enrichment of the probe sets on the Affymetrix array.

### EXAMPLE 3

Step 3: Validation. Next, validation for changes in a subject having clinically severe mood disorders (depression, mania) was carried out. These prioritized biomarkers, in a demographically matched cohort of (n=30 clinically severe depression, n=17 clinically severe mania), were assessed for which markers were stepwise changed in expression from clinically severe depression in validation cohort to low mood in discovery cohort to high mood in discovery cohort to clinically severe mania in the validation cohort. 4633 probe sets were not stepwise changed, and 1737 were stepwise changed. Of these, 291 probe sets were determined to be nomially significant. The 291 probe sets result represents approximately a 188-fold enrichment of the probe sets on the Affymetrix array.

The scores from the first three steps were added into an overall convergent functional evidence (CFE) score (FIG. 1), which generated a list of 23 mood disorder biomarkers (n=23 genes, 26 probe sets), that had a CFE score as good as or better than the score of the biomarker SLC6A4. The score determined for SLC6A4 was used as a positive control and a threshold for determining if a putative biomarker should be included in the diagnostic panel. The 23 mood disorder blood biomarkers identified represents approximately an over 2,000-fold enrichment of the probe sets on the Affymetrix array. These 23 mood disorder biomarkers (Table 5) were carried forward into additional analyses for biological understanding and for clinical utility, as well as tested in the independent cohort step.

Biological Understanding. Biological Pathways. Biological pathway analyses were conducted using the mood disorder biomarkers for mood (n=23 genes, 26 probe sets), which suggests involvement of circadian rhythm, neurotrophic, and cell differentiation functions as well as serotonergic and glutamatergic signaling, relating to mood as reflecting activity and growth (Table 2A). Depression, along with weight gain, were the principal diseases identified by the pathway analyses using the Database for Annotation, Visualization and Integrated Discovery (“DAVID”), and the top diseases identified using the QIAGEN Ingenuity Pathway Analysis were neurological and psychological disorders and cancer.

Circadian. A number of the mood disorder biomarkers identified herein have biological roles that are related to the circadian clock (e.g., 8 out of 23 genes). Circadian clock abnormalities may be related to mood disorders (Carthy, M. J. et al., “Cellular circadian clocks in mood disorders,” J. Biol. Rhythms 27: 339-352 (2012); Le-Niculescu, H., et al., “Phenomic, convergent functional genomic, and biomarker studies in a stress-reactive genetic animal model of bipolar disorder and co-morbid alcoholism,” American journal of medical genetics. Part B, Neuropsychiatric genetics: the official publication of the International Society of Psychiatric Genetics 147B: 134-166 (2008)).

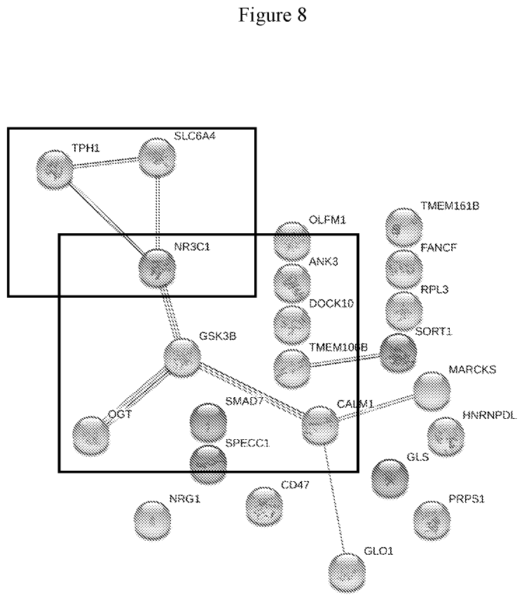

Networks and Interactions. Using the STRING Protein-Protein Interaction Networks Functional Enrichment Analyses of the mood disorder biomarkers, interacting protein groups were identified. In particular, NR3C1 ((Nuclear Receptor Subfamily 3, Group C, Member 1 (Glucocorticoid Receptor)) is at the overlap of a network containing SLC6A4 and TPH1, and one centered on GSK3B that also contains OGT and CALM1 (FIG. 8). The networks identified and shown in FIG. 8 support the biological significance of the identified biomarkers in the context of mood disorders and their therapeutic targeting.

Evidence for involvement in other disorders. CFG analyses were conducted using the biomarkers with highest expression (Table 7), which suggest that many, if not all, of the mood disorder biomarkers may be involved in other psychiatric disorders, providing a basis for co-morbidity and increased vulnerability.

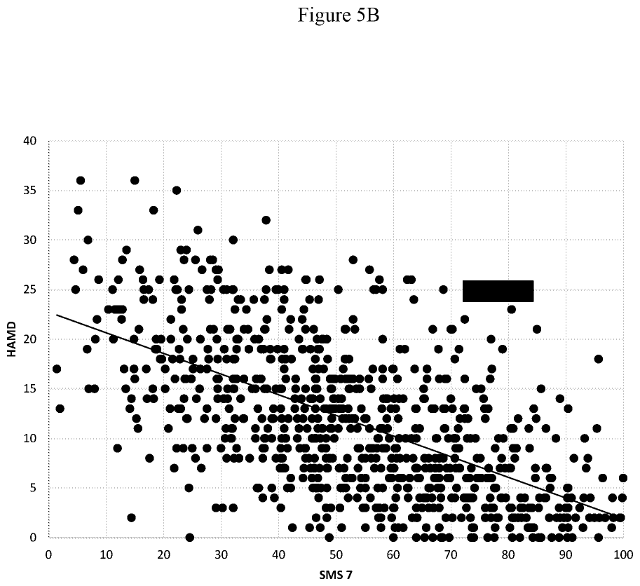

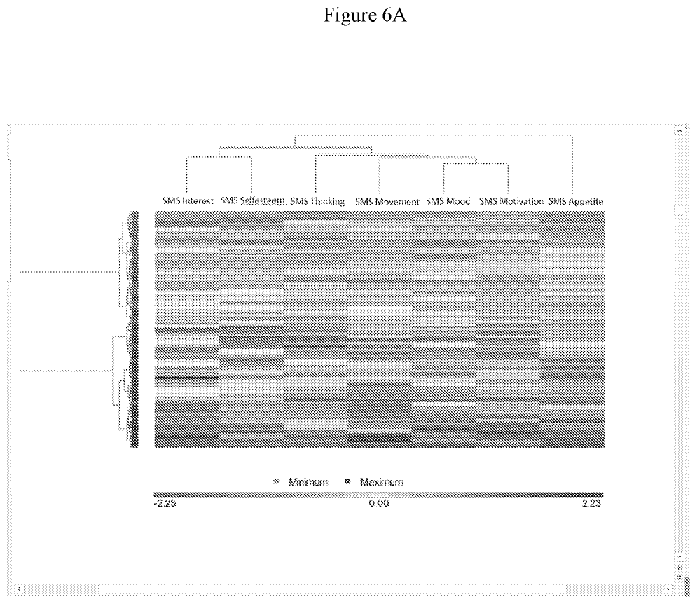

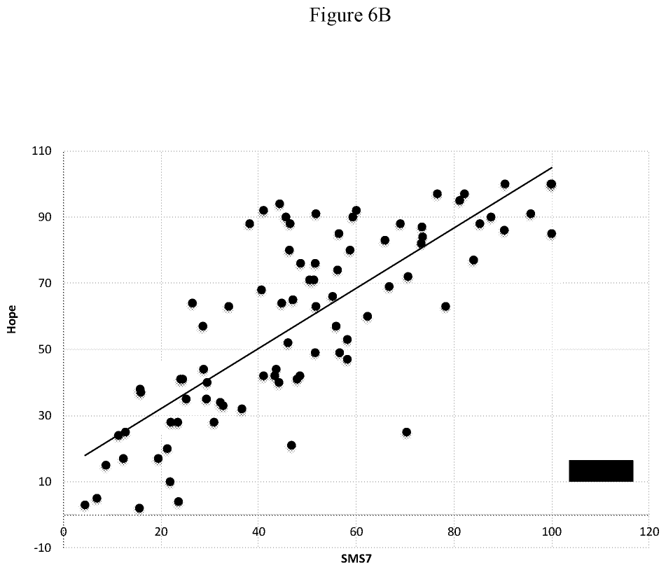

Phenomenology. The mood SMS-7 consists of seven items (FIG. 5A). Clustering analysis shows the structure of mood symptoms (FIG. 6A). “Mood” and “Motivation to do things” were most closely related to one another, followed by “Movement activity” and “Thinking activity.” “Self-esteem” and “Interest” in pleasurable activities are more distant from each other and therefore deemed to be less related to one another. “Appetite” is the most distant, and therefore least related to the other items on the scale depicted in FIG. 5A. Mood reflects and underlies, in essence, if an individual is motivated to get on with life/activities or not. Germane to that, as shown herein, SMS7 correlates well with a visual analog scale for Hope. Using essentially the same scale which was used in FIG. 5, the visual analog scale for hope is depicted in FIG. 6B.

### EXAMPLE 4

Step 4: Testing for Clinical Utility.

Testing for assessment and predictive ability in independent cohorts of 23 mood disorder biomarkers was carried out (composed of the top scoring biomarkers after the first three steps: discovery, prioritization, and validation) (FIG. 1 and Table 5). The 23 mood disorder biomarkers were tested individually, in completely independent test cohorts of psychiatric disorder subjects. The individual assessment of the 23 biomarkers led to the identification of universal (gender neutral) biomarkers whose expression levels are altered in individuals with mood disorders across gender and diagnoses. Such biomarkers include for example: NRG1, DOCK10, and SLC6A4.

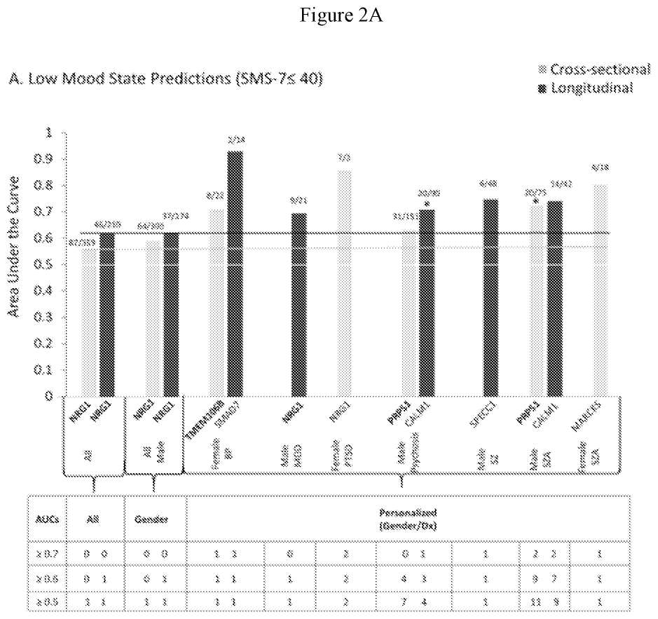

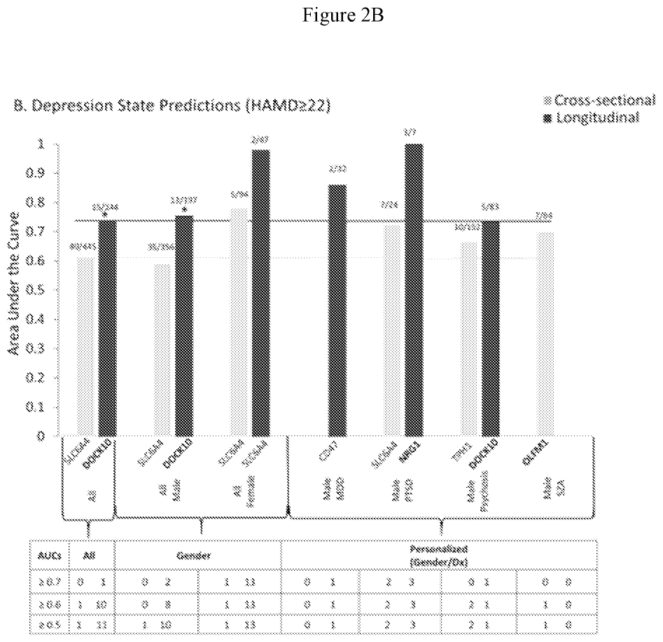

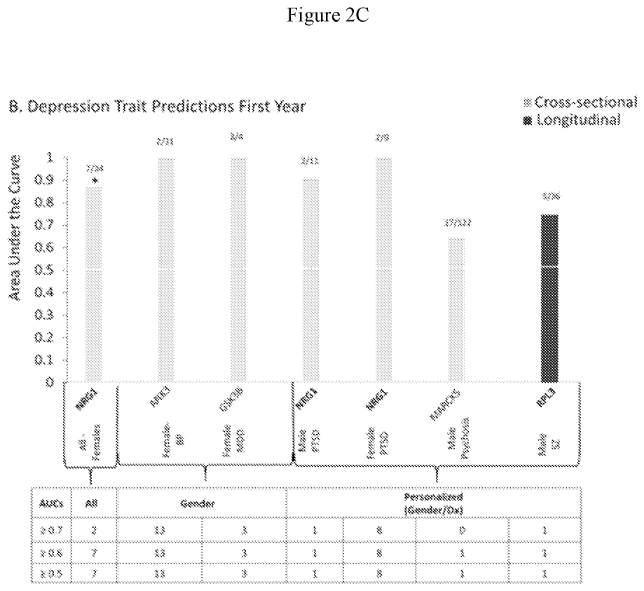

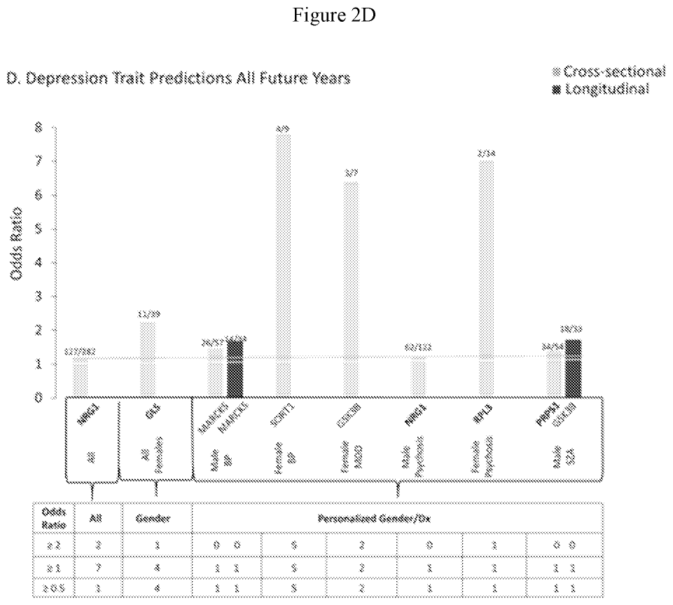

The predictive ability of each of the 23 biomarkers was further studied in subjects in the independent cohort, grouped by gender and by psychiatric diagnosis. The universal approach was compared to this more personalized approach and showed that the personalized approach permits enhanced precision of predictions for different biomarkers (FIGS. 2A-2D and Tables 3A-3C). The bar graphs in FIGS. 2A-2D show prioritized predictive biomarkers for depression and mania, by state and trait, in each group. These prioritized predictive biomarkers were assessed based on the mood disorder biomarkers from each of the Steps 1-3 (Discovery, Prioritization, Validation-Bold) (n=26 All markers are nominally significant p<0.05). The tables underneath each of FIGS. 2A-2D display the actual number of biomarkers for each group whose ROC AUC p-values (FIG. 2A-2C) and Cox Odds Ratio p-values (FIG. 2D) are at least nominally significant. Some gender and diagnosed groups are not displayed in these figures as graphs from these groups did not include any significant biomarkers.

The cross-sectional area under the curve is based on levels measured in an individual subject determined during one visit. The Longitudinal area under the curve is based on levels measured at multiple patient visits. These values integrate levels measured at the most recent visit, maximum levels, slope determined in the most recent visit, and maximum slope. Dividing lines represent the cutoffs for a test performing at chance levels (white), and at the same level as the prioritized biomarkers for all subjects in cross-sectional (gray) and longitudinal (black)-based predictions. All biomarkers perform better than chance. Biomarkers performed better when personalized by gender and diagnosis, particularly in females. “**” indicates survived Bonferroni correction for the number of candidate biomarkers tested.

For low-mood state assessment across all subjects in the independent test cohort, the most common biomarker was NRG1 (neuregulin 1), which increased in expression in a low mood, with an AUC of 62% (p=6.8E-03), and 64% (p=3.5E-02) for assessing clinical depression state. NRG1 also had a Cox regression Odds Ratio of 1.5 (p=3.77E-02) for all future hospitalizations for depression in males with depression. NRG1 also had a Cox regression Odds Ratio of 1.17 (p=2.5E-02) for predicting all future hospitalizations for depression, and an AUC of 87% (p=1.1E-03) for predicting first-year hospitalizations for depression in females. Moreover, in the opposite direction, when a high-mood state is assessed or identified across all subjects, NRG1 has a modest AUC of 58% (p=1.4E-02), but is a robust predictor of all future hospitalizations for mania in patients with psychotic disorders (Cox regression OR of 2.7 (p=3.3E-02)).

NRG1 is known as a membrane glycoprotein that mediates cell-cell signaling and plays a critical role in the activity, growth and development of multiple organ systems. It is a direct ligand for ERBB3 and ERBB4 tyrosine kinase receptors, resulting in ligand-stimulated tyrosine phosphorylation and activation of the ERBB receptors. Activity and trophicity of tissues may be involved with mood (Niculescu, A. B. Genomic studies of mood disorders—the brain as a muscle? Genome Biol 6: 215 (2005)).

For assessment of clinical depression state in the independent test cohort, DOCK10 (dedicator of cytokinesis 10) decreased in expression in low-mood assessed subjects, had an AUC of 73% (p=1.17E-03) across all subjects, and 75% (p=1.05E-03) in males, surviving Bonferroni correction for all 26 biomarkers tested. DOCK10 also had an AUC of 95% (p=1.52E-02) for males with posttraumatic stress disorder (PTSD). DOCK10 had a Cox regression Odds Ratio of 1.9 (p=3.93E-02) for predicting all future hospitalizations for depression in females. Moreover, in the opposite direction for assessing a high-mood state, DOCK10 had an AUC of 70% in females (p=2.63E-02), and 100% (p=9.18E-04) in females with bipolar disorder (Table 3B).

DOCK10 is a guanine nucleotide-exchange factor (GEF) that activates CDC42 and RAC1 by exchanging bound GDP for free GTP. It is essential for dendritic spine morphogenesis in Purkinje cells and in hippocampal neurons, via a CDC42-mediated pathway.

For a clinical depression state assessment in the independent test cohort across all subjects, SLC6A4 increased in expression in subjects having a low mood, had an AUC of 61% (p=1.1E-02) if measured cross-sectionally, and an AUC of 66% (p=1.78E-02) if measured longitudinally. SLC6A4 was more accurate in female subjects having an AUC of 78% (p=1.8E-02) if measured cross-sectionally, and an AUC of 98% (p=1.1E-02) if measured longitudinally. Moreover, when SLC6A4 was used for detecting high moods, for predicting future hospitalizations for mania in the first year, across all subjects, the biomarker had an AUC of 74% (p=3.3E-03). SLC6A4 had an even better accuracy in male subjects diagnosed as bipolar, with an AUC of 77% (p=1.3E-02). The product of the SLC6A4 gene is a serotonin transporter, which is a target of serotonin reuptake inhibitors used to treat depression, as well as anxiety and stress disorders. Of note, it is known that individuals with bipolar disorder treated with SSRIs, especially in monotherapy, can switch into mania.

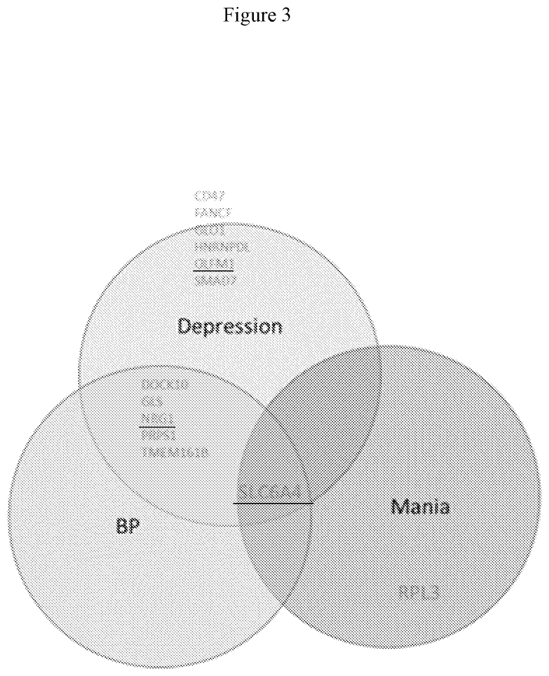

As shown in FIG. 3, there is an overlap between the biomarkers expressed in depression, bipolar disorder, and mania. As disclosed herein and shown in FIG. 3, RPL3 may be a target for the treatment of mania with less risk of inducing depression. Six biomarkers (i.e., CD47, FANCF, FARSB, GLO1, HNRNPDL, OLFM1, and SMAD7), may be targets individually or together for the treatment of depression with less risk of inducing mania. Further, another six biomarkers (i.e., DOCK10, GLS, NRG1, PRPS1, TMEM161B, and SLC6A4) may be targeted to rapidly treat depression but may induce mania in the treated subject; in patients having these 6 biomarkers, treatment of the depression may be coupled with a mood stabilizer or antipsychotic to yield an effective therapy through a combination of therapeutics to avoid inducing mania. In FIG. 3, underlining indicates an increase in expression in low mood, and no underlining indicates an increase in expression in high mood.

STRING Protein-Protein Interaction Networks Functional Enrichment Analyses revealed groups of interacting proteins for low mood/depression/hospitalizations across all subjects in the independent test cohort (n=23 genes, 26 probe sets). (FIG. 8). The networks thus identified provide insight into both the biological significance and areas for targeted and/or repurposed therapeutics.

As represented in FIG. 5, the mood assessment test SMS-7 consists of seven items. The overall SMS-7 score is generated by averaging the scores determined for each score of the seven items. See, e.g., Mood Subscale (SMS, Simplified Mood Scale) of the Simplified Affective State Scale (SASS) (Niculescu et al. 2006, 2015). The clustering analysis presented herein revealed the structure of mood symptoms (FIG. 5).

As represented in FIG. 6, using PhenoChipping Clustering of items of the VAS, mood is most closely related to a Motivation to do things, followed by Movement activity and Thinking activity. Self-esteem and Interest in pleasurable activities are more distant and related to each other. Appetite is the most distant, and least related to the other six items. Mood reflects and underlies, in essence, if an individual is motivated to get on with life/activities or not. This analysis was performed on quantitative phenomic data as had been performed on gene expression data, using a two-way unsupervised hierarchical clustering using the Discovery cohort data (n=134 visits, from 44 subjects).

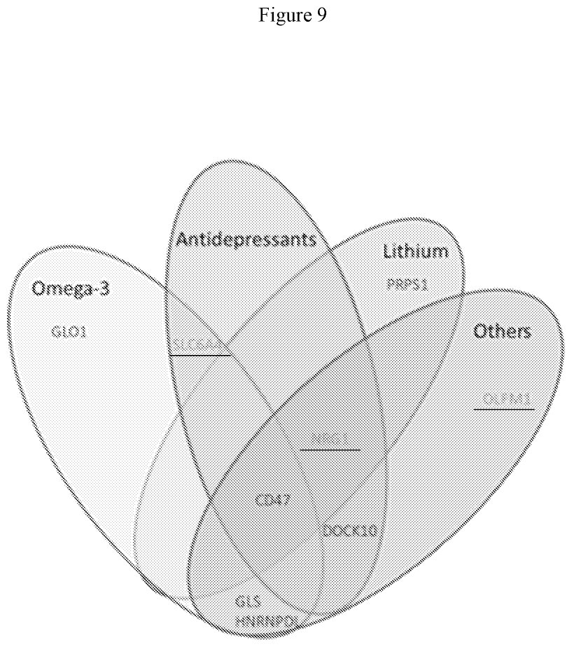

Pharmacogenomics. A number of individual mood disorder biomarkers are known to be modulated by medications in current clinical use for treating depression, such as lithium (NRG1, PRPS1, CD47), antidepressants (SLC6A4, DOCK10, NRG1, CD47) and the nutraceutical omega-3 fatty acids (GLO1, SLC6A4, CD47, GLS, HNRNPDL) (FIG. 9 and Tables 3 and 8). In particular, NRG1 is at the overlap of lithium and antidepressants, and CD47 is at the overlap of all three treatments (FIG. 9). Omega-3 fatty acids may be a widely deployable preventive treatment, with minimal side-effects, including in women who are, or may become, pregnant.

FIG. 9 shows the pharmacogenomics for expression (BioM12). Multiple biomarkers of depression show evidence of being modulated by existing drugs known generally to induce an opposite effect of these drugs on depression/low mood. See also Table 8. Underlining in Table 8 indicates an increase in expression in low mood; text lacking underlining indicates an increase in high-mood expression. These identified biomarkers may be used to target treatments to different patients, and to measure the response to that treatment. The higher the proportion/percentile of biomarkers for a certain drug/class, the more specific the drug is indicated for effective treatment. When biomarkers for multiple different drug/classes are changed in an individual, a prioritization based on the proportion/percentile of biomarkers for each class may be used to choose the drug or combination of drugs (targeted rational polypharmacy).

New drug discovery/repurposing. As shown in Table 4, bioinformatic analyses using the gene expression signature of panels of mood disorder biomarkers for low mood/depression identified new potential therapeutics for depression, such as the beta-blocker f3-blocker and serotonin 5HT1A presynaptic receptor antagonist pindolol, the PPAR-alpha activator and lipid lowering agent ciprofibrate, the PPAR-γ activator and anti-diabetic pioglitazone, and the anticholinergic and antispasmodic adiphenine. The bioinformatic analyses also identified the natural compounds asiaticoside and chlorogenic acid. Asiaticoside is a triterpenoid component derived from Centella asiatica (Gotu Kola), used in antioxidant, anti-inflammatory, immunomodulatory, and wound healing applications. Chlorogenic acid is an antioxidant, polyphenol found in coffee.

The biomarkers identified herein may be used to target treatments to different patients, and to measure response to that treatment. The higher the proportion/percentile of biomarkers for a certain drug/class, the more indicated that drug would be for treatment. When biomarkers for multiple different drug/classes are changed in an individual, a prioritization based on the proportion/percentile of biomarkers for each class could be used to choose the drug or combination of drugs (targeted rational polypharmacy).

### Convergent Functional Evidence (CFE)

Tables 3A-3C list Convergent Functional Evidence (CFE) for top biomarkers for: Table 3A: Low Mood/Depression, Table 3B: Bipolar Mood Disorders, and Table 3C: High Mood/Mania based on the totality of evidence from the previously disclosed studies (Discovery, Prioritization, Validation, and Testing). In Tables 3A-3C, DE means differential expression, AP means Absent/Present, NS means Non-stepwise in validation, and bolded names of genes indicate nominally significant at Step 3 validation. For Step 4 Predictions, C means cross-sectional (using levels from one visit) and L means longitudinal (using levels and slopes from multiple visits). In ALL, by Gender, and personalized by Gender and Diagnosis, score for predictions: 3 pts if in ALL, 2 pts Gender, 1 pts Gender/Dx. Underlined indicates prioritized predictive biomarker for that phenotype and population. M means Males; F means Females; MDD means depression; BP means bipolar; SZ means schizophrenia; SZA means schizoaffective; PSYCHOSIS means schizophrenia and schizoaffective combined; and PTSD means post-traumatic stress disorder.

Table 3A contains biomarkers for Low Mood/Depression (n=12 genes, 13 probe sets, using as a cutoff the score for SLC6A4). Table 3B contains biomarkers for Bipolar Mood Disorders (n=6 genes, using as a cutoff the score for SLC6A4), genes which are also found in the list of biomarkers for depression in Table 3A. Table 3C contains biomarkers for High Mood/Mania (n=2 genes, using as a cutoff the score for SLC6A4). RPL3 is not overlapping with the list of top biomarkers for depression in Table 3A.

The mood disorder biomarkers (n=23), were tabulated into a convergent functional evidence (CFE) score using all the evidence from discovery (up to 6 points), CFG prioritization (up to 12 points), validation (up to 6 points), and testing. Testing includes evaluation of ability to correctly predict in independent cohorts the following: state low mood, state clinical depression, trait first-year hospitalization with depression, trait all future hospitalizations with depression, as well as state high mood, state clinical mania, trait first-year hospitalization with mania, trait all future hospitalizations with mania—up to 3 points each if significantly predictive in all subjects, 2 points if predictive by gender, and 1 point if predictive in gender/diagnosis. The total score can be up to 48 points: 36 of the points are obtained from collected data and 12 points are obtained from literature data used for CFG. The new empirical data was weighed three times more than the literature data, as it is functionally related to mood in 3 independent cohorts (discovery, validation, testing). The goal was to highlight, based on the totality of the data and of the evidence in the field to date, biomarkers that have all around evidence, i.e. that can track mood, have convergent evidence for involvement in mood disorders, and predict mood state and future clinical events.

The six blood biomarkers with the strongest overall convergent functional evidence (CFE) for tracking and predicting both depression and mania (i.e., bipolar mood disorders) identified after the first four steps of the process described above were NRG1, DOCK10, GLS, PRPS1, TMEM161B, and SLC6A4. For example, NRG1 decreased in expression in high mood, survived discovery, prioritization and validation, indicating that it may be a better predictor for low mood/depression, especially when personalized by gender and diagnosis, than for high mood/mania (see also Tables 3 and 5).

### Universal Predictor of Mood (Up-Mood)

CFI-BP (Convergent Functional Information of Bipolar Disorder) severity based on scores of 1-10. Generating scores for individuals included assigning points based on the following inputs.

1. Medications
M1. Tried on more than two different mood stabilizing medications (1 pt.)
M2. Was/is on lithium or divalproex (1 pt.)
M3. Was/is on antipsychotics (1 pt.)
   Summing the points assigned to M1, M2, and M3 produces the Medication Score (0-3).

2. Severity of Illness

Factors in this aspect of the analysis include the following:
I1. Multiple hospitalizations on inpatient psychiatric units (1 pt.)
I2. Hospitalized in a State Hospital, or for more than 21 days (1 pt.)
I3. YMRS greater than 15 or HAM-D greater than 20 at time of testing (1 pt.)
I4. No history of enrollment in substance abuse programs or treatments (1 pt.) Summing the points assigned to I1, I2, I3, and I4 produces the Severity Score (0-4).

3. Social Functioning

Factors in this aspect of the analysis include the following:
F1. On 100% disability (1 pt.)
F2. Was/is on commitment (has conservator/payee) (1 pt.)
F3. Has been in bankruptcy or lost home to foreclosure or married more than 3 times (1 pt.).
   Summing the points assigned to F1, F2, and F3 produces the Social Functioning Score (0-3). The Total Score for an individual is the sum of the Medication, Severity of Illness, and Social Functioning Scores. The minimum score is 0 and the maximum score is 10.

Referring now to Table 14. Predictions using an apriorism algorithm combining as predictors BioM26 with mood (SMS7) and with clinical severity of bipolar disorder (CFI-BP) in all subjects in the independent test cohort. Cross-sectional analyses.

### Materials and Methods

Three independent cohorts were used. Cohort 1: discovery (a longitudinal psychiatric subject's cohort with diametric changes in mood state from at least two consecutive testing visits); Cohort 2: validation (an independent psychiatric subject's cohort with clinically severe depression or mania); and Cohort 3: testing (an independent psychiatric subject's test cohort for predicting mood state, clinical depression or mania, and for predicting future hospitalizations for depression or mania) (FIG. 1A). The demographics of each of the 3 cohorts are listed in Table 1 (BP means bipolar; MDD means Major depressive disorder; SZA means schizoaffective disorder; SZ means schizophrenia; and PTSD means post-traumatic stress disorder).

### Subjects

Live psychiatric subjects were part of a larger longitudinal cohort of adults that undergo continuous collecting. (see Niculescu, A. B., et al., Understanding and predicting suicidality using a combined genomic and clinical risk assessment approach. Mol. Psychiatry 20: 1266-1285 (2015); Levey, D. F., et al., Towards understanding and predicting suicidality in women: biomarkers and clinical risk assessment. Mol. Psychiatry 21: 768-785 (2016); Le-Niculescu, H., et al., Discovery and validation of blood biomarkers for suicidality. Mol. Psychiatry 18: 1249-1264 (2013)). Subjects were recruited primarily from the patient population at the Indianapolis Veterans Administration Medical Center. All subjects understood and signed informed consent forms detailing the research goals, procedure, caveats and safeguards, per Institutional Review Board (IRB) approved protocol.

Subjects completed diagnostic assessments by structured clinical interviews (Diagnostic Interview for Genetic Studies, MINI, or SCID). They had an initial testing visit in the lab or in the inpatient psychiatric unit, followed by up to six testing visits, 3-6 months apart or whenever a new psychiatric hospitalization occurred. At each testing visit, they received a series of psychiatric rating scales and their blood was drawn. The rating scales included the Hamilton Rating Scale for Depression-17 (HAMD), the Young Mania Rating Scale (YMRS), and a visual analog scale for assessing mood state (SMS-7). The SMS-7 score is the average of seven items (FIG. 5) and is part of the Simplified Affective State Scale (SASS) (see Niculescu, A. B., et al., PhenoChipping of psychotic disorders: a novel approach for deconstructing and quantitating psychiatric phenotypes. American journal of medical genetics. Part B, Neuropsychiatric genetics: the official publication of the International Society of Psychiatric Genetics 141B: 653-662 (2006); Niculescu, A. B., et al., Understanding and predicting suicidality using a combined genomic and clinical risk assessment approach. Mol. Psychiatry 20: 1266-1285 (2015); Levey, D. F., et al., Towards understanding and predicting suicidality in women: biomarkers and clinical risk assessment. Mol. Psychiatry 21: 768-785 (2016); Niculescu, A. B., et al., Precision medicine for suicidality: from universality to subtypes and personalization. Mol. Psychiatry 22: 1250-1273 (2017)). SMS-7 integrates on a continuum in a quantitative fashion clinical symptoms for depression and mania, and provides a score for mood state at a particular moment in time. It has good face validity based on DSM criteria, and correlates inversely with HAMD (see Niculescu, A. B., et al., PhenoChipping of psychotic disorders: a novel approach for deconstructing and quantitating psychiatric phenotypes. Amer. J. Med. Gen. Part B, Neuropsychiatric genetics: The Official publication of the International Society of Psychiatric Genetics 141B: 653-662 (2006)).

A software application (app) version of SASS was created and used. In addition to seven items measuring mood, there were four items measuring anxiety (SAS-4). The PANSS Positive scale was also used, which is a scale that measures positive psychotic symptoms. SAS-4 and PANSS Positive may be used to define subtypes of low mood, as shown in the Discovery cohort (FIG. 7).

FIG. 7 depicts various subtypes: of low mood, determined with a number of low mood visits (n=63) in the 44 subjects in Discovery cohort, Z-scored by gender and diagnosis. The X-axis reflects subject visits. The Y-axis reflects measured of anxiety and psychosis. These results demonstrate that different combinations of biomarkers are predictive of different patient subset populations all of whom exhibit low mood/depression.

At each visit, whole blood (5 mL) was collected from each subject between two RNA-stabilizing PAXgene tubes, labeled with an anonymized ID number, and stored at −80° C. in a locked freezer until the time of future processing. Whole-blood RNA was extracted for microarray gene expression studies from the PAXgene tubes, as detailed below.

For these studies, the within-subject discovery cohort, from which the biomarker data were derived, consisted of 44 subjects (30 males, 14 females) with psychiatric disorders and multiple testing visits, who each had at least one diametric change in SMS-7 mood scores from low mood (SMS-7≤40) to high mood (SMS-7≥60), or vice versa, from one testing visit to another. There were 4 subjects with 6 visits each, 6 subjects with 4 visits each, 18 subjects with 3 visits each, and 16 subjects with 2 visits each resulting in a total of 134 blood samples for subsequent gene expression microarray studies (FIGS. 1A-1F, Table 1).

The independent validation cohort, in which the mood disorder biomarker findings were validated for being even more changed in expression, consisted of 39 male and 8 female subjects having a clinically severe mood disorder (n=30 depression as measured by HAMD scores ≥22, and n=17 mania as measured by YMRS scores ≥20), and a concordant low mood, respectively high mood SMS-7 scores (Table 4A1 and FIG. 5). Table 4A1 depicts the direction of High Mood (probe sets for 16 increased and 72 decreased biomarkers were present in HG-U133A array used by CMAP).

The independent test cohort for predicting low-mood state (SMS-7≤40) and high-mood state (SMS-7≥60) consisted of 153 male and 37 female subjects with psychiatric disorders, demographically matched with the discovery cohort, with one or multiple testing visits in our lab, with either low mood, intermediate mood, or high mood states (FIGS. 1A-1F and Table 4A1).

The independent test cohort for predicting clinical depression state (HAMD≥22) consisted of 181 male and 45 female subjects with psychiatric disorders, demographically matched for age, with one or multiple testing visits, with either low, intermediate, or high HAMD scores. The independent test cohort for predicting a clinical mania state (YMRS≥20) consisted of 73 males and 24 female subjects with psychiatric disorders, demographically matched for age, with one or multiple testing visits, with either low, intermediate, or high YMRS scores (FIGS. 1A-1F and Table 1).

The test cohorts for predicting future hospitalizations with accompanying depression, and future hospitalizations with accompanying mania (FIGS. 1A-1F and Table 1), were a subset of the independent test cohort for which there was a longitudinal follow-up made with the sued of electronic medical records. The subjects' subsequent number of hospitalizations with depression, or with mania, was tabulated from electronic medical records.

The subjects in the discovery cohort were all diagnosed with various psychiatric disorders (Table 1) and had various medical co-morbidities. Their medications were listed in their electronic medical records and documented by us at the time of each testing visit. Medications can have a strong influence on gene expression. To correct for this in the disclosed results, the differentially expressed genes were each based on within-subject analyses, which factor out not only genetic background effects but also minimizes medication effects, as the subjects rarely had major medication changes between visits. Moreover, there was no consistent pattern of any particular type of medication, as the subjects were on a wide variety of different medications, psychiatric and non-psychiatric. Furthermore, the independent validation and testing cohorts' gene expression data was Z-scored by gender and diagnosis before being combined, to normalize for any such effects. Some subjects may have been non-compliant with their treatment and may thus have changes in medications or drug of abuse not reflected in their medical records. The prioritization step that occurred after discovery was based on a field-wide convergence with literature that includes genetic data and animal model data, that are unrelated to medication effects.

### RNA Extraction

Whole blood (2.5 ml) was collected from each subject and placed into each PaxGene tube by routine venipuncture. PaxGene tubes contain proprietary reagents for the stabilization of RNA. RNA was extracted and processed as previously described (Niculescu, A. B., et al., Understanding and predicting suicidality using a combined genomic and clinical risk assessment approach. Mol. Psychiatry 20: 1266-1285 (2015); Levey, D. F., et al., Towards understanding and predicting suicidality in women: biomarkers and clinical risk assessment. Mol. Psychiatry 21: 768-785 (2016)).

### Microarrays

Microarray work was carried out using previously described methodology. (see Niculescu, A. B., et al. Understanding and predicting suicidality using a combined genomic and clinical risk assessment approach. Mol. Psychiatry 20: 1266-1285 (2015); Levey, D. F., et al. Towards understanding and predicting suicidality in women: biomarkers and clinical risk assessment. Mol. Psychiatry 21: 768-785 (2016); Le-Niculescu, H., et al. Discovery and validation of blood biomarkers for suicidality. Mol. Psychiatry 18: 1249-1264 (2013); Niculescu, A. B., et al. Precision medicine for suicidality: from universality to subtypes and personalization. Mol. Psychiatry 22: 1250-1273 (2017)). All genomic data was normalized (RMA for technical variability, then z-scoring for biological variability), by gender and psychiatric diagnosis, before being combined and analyzed.

### Biomarkers

Step 1: Discovery

A subject's score from a visual-analog scale (SMS-7) scale was assessed at the time of the subject's blood collection. Gene expression differences were analyzed between visits with low mood (low mood was defined as a score of 0-40) and visits with high mood (high mood was defined as a score of 60-100), using a powerful within-subject design, then an across-subjects summation (FIG. 1A).

The data obtained from the visits and blood analyses were analyzed in two ways: the Absent-Present (AP) approach, and a differential expression (DE) approach, as previously described (Niculescu, A. B., et al. Understanding and predicting suicidality using a combined genomic and clinical risk assessment approach. Mol. Psychiatry 20: 1266-1285 (2015); Levey, D. F., et al. Towards understanding and predicting suicidality in women: biomarkers and clinical risk assessment. Mol. Psychiatry 21: 768-785 (2016); Le-Niculescu, H., et al. Discovery and validation of blood biomarkers for suicidality. Mol. Psychiatry 18: 1249-1264 (2013)). The AP approach may capture turning on and off of genes, and the DE approach may capture gradual changes in expression. In brief, all Affymetrix microarray data were imported as CEL files into the Partek Genomic Suites 6.6 software package (Partek Incorporated, St. Louis, MO, USA). Using only perfect match values, a robust multi-array analysis (RMA) was performed by gender and diagnosis, background noise from the gene chip array data was corrected for using quantile normalization and a median polish probe set summarization of all chip derived data, to obtain the normalized expression levels of all probe sets for each chip. To establish a list of differentially expressed probe sets, a within-subject analysis was conducted using a fold change in biomarker expression of at least 1.2 between consecutive high- and low-mood visits for each individual subject. Probe sets that have a 1.2−fold change are then assigned either a +1 (increased in high mood) or a −1 (decreased in high mood) in each comparison. Fold changes between 1.1 and 1.2 are given 0.5, and fold changes less than 1.1 are given 0. These values were then summed for each probe set across all the comparisons and subjects, yielding a range of raw scores. The probe sets above the 33.3% of raw scores were carried forward in analyses (FIG. 1), and received an internal score of 2 points, probe sets above 50% received 4 points, and probe sets above 80% received 6 points. R scripts commands were automated and conducted on all of these large dataset analyses in bulk; and these values were checked against human manual scoring. The Gene Symbol for the probe sets were identified using NetAffyx (Affymetrix) for Affymetrix HG-U133 Plus 2.0 GeneChips, followed by GeneCards to confirm the primary gene symbol. In addition, for those probe sets that were not assigned a gene symbol by NetAffyx, GeneAnnot (https://genecards.weizmann.ac.il/geneannot/index.shtml) was used, or if need be, UCSC (https://genome.ucsc.edu), to obtain gene symbol for these uncharacterized probe sets, followed by GeneCard. Biomarker genes were then scored using the disclosed manually curated CFG databases as described below (FIG. 1D).

Step 2: Prioritization using Convergent Functional Genomics (CFG)

### Databases.

Manually curated databases of the human gene expression/protein expression studies (postmortem brain, peripheral tissue/fluids: CSF, blood and cell cultures), human genetic studies (association, copy number variations and linkage), and animal model gene expression and genetic studies were established for all studies published to-date on psychiatric disorders. The databases include only primary literature data and do not include review papers or other secondary data integration analyses to avoid redundancy and circularity. These large and constantly updated databases were used in the CFG cross validation and prioritization platform (FIG. 1D). For this study, data from 1600 papers on mood disorders were present in the databases at the time of the CFG for mood disorders analyses (June 2018) (human genetic studies-759, human brain studies-246, human peripheral tissue/fluids-359, non-human genetic studies-47, non-human brain/studies-167, non-human peripheral tissue/fluids-22). A computerized CFG Wizard automated and scored, in bulk, large lists of genes by integrating evidence from these large databases, checked against manual scoring, as previously described (Niculescu, A. B., et al. Precision medicine for suicidality: from universality to subtypes and personalization. Mol. Psychiatry 22: 1250-1273 (2017)). Analyses were performed as previously described (Niculescu, A. B., et al. Understanding and predicting suicidality using a combined genomic and clinical risk assessment approach. Mol. Psychiatry 20: 1266-1285 (2015); Levey, D. F., et al. Towards understanding and predicting suicidality in women: biomarkers and clinical risk assessment. Mol. Psychiatry 21: 768-785 (2016)).

Table 6 depicts CFG for Mood, as used in Step 2, Prioritization for Biomarkers for Low Mood/Depression (BioM12 Depression), and Mania (RLP3). The gene name without underlining indicates an increase in expression (I) in High Mood; a gene name that is underlined indicates a decrease in expression in High Mood (D). DE—differential expression, AP—Absent/Present.

### Step 3: Validation Analyses

The mood disorder biomarker genes identified in from Step 2 of this study (total CFG score of 6 or above), were analyzed for stepwise changes in expression from the clinically depressed validation group to the low mood discovery group to the high mood discovery group to the clinically manic validation group. A CFG score of 6 or greater reflected an empirical cutoff of 33.3% of the maximum possible total internal and external CFG score of 18. This methodology h permitted the inclusion of potentially novel genes that exhibited maximal internal score for the Discovery step of 6, but no external evidence scores from the Prioritization step for any potentially novel genes identified using this method of analysis. Subjects with low mood and subjects with high mood from the discovery cohort who did not have clinical depression or mania were used, along with the independent validation cohort (n=47).

The AP-derived and DE-derived lists of genes were combined, and the gene expression data corresponding to them was used for the validation analysis. The four groups (i.e., clinical depression, low mood, high mood, and clinical mania) were assembled out of Affymetrix CEL data that was RMA normalized by gender and diagnosis. The log transformed expression data was transferred to an Excel sheet. The values were Z-scored by gender and diagnosis. The bioinformatic software package Partek was utilized for performing statistical analyses, including a one-way ANOVA for the stepwise changed probe sets. Stringent Bonferroni corrections was performed for all the probe sets tested (stepwise and non-stepwise) as reflected in FIG. 1E.

The mood disorder biomarkers, after the first three steps, were identified by adding the scores from the first three steps of this example into an overall convergent functional evidence (CFE) score (FIG. 1). This resulted in a list of 23 mood disorder biomarkers (n=23 genes, 26 probe sets), that had a CFE score as good as or better than SLC6A4, which was used as a positive control and threshold. The 23 identified mood disorder biomarkers were carried forward into additional analyses for biological understanding and for clinical utility.

Circadian Rhythm Gene Database. A database of genes associated with circadian rhythm function was compiled using a combination of review papers (Zhang, E. E., et al. A genome-wide RNAi screen for modifiers of the circadian clock in human cells. Cell 139: 199-210 (2009); McCarthy, M. J. et al. Cellular circadian clocks in mood disorders. Journal of biological rhythms 27: 339-352 (2012)) and searches of existing databases CircaDB (http://circadb.hogeneschlab.org), GeneCards (http://www.genecards.org), and GenAtlas (http://genatlas.medecine.univ-paris5.fr). Using the data derived from these sources, 1468 genes were identified that demonstrated circadian rhythm functioning. These genes were further classified into “core” circadian genes, (i.e. those genes that are the main engine driving circadian function (n=18)), “immediate” clock genes (i.e. the genes that directly input or output to the core clock (n=331)), and “distant” clock genes (i.e. genes that directly input or output to the immediate clock genes (n=1119)).

Pathway Analyses. IPA (Ingenuity Pathway Analysis, version 24390178, Qiagen), David Functional Annotation Bioinformatics Microarray Analysis (National Institute of Allergy and Infectious Diseases) version 6.7 (August 2016), and Kyoto Encyclopedia of Genes and Genomes (KEGG) (through DAVID) were used to analyze the biological roles of the identified genes, including the primary canonical pathways and diseases the genes appeared to have a role (Tables 2A-2B). The analyses were performed for the 23 unique genes related to the mood disorder biomarkers after the discovery, prioritization, and validation.

Network Analyses. STRING Interaction network (https://string-db.org) networks analyses were performed by entering the 23 mood disorder biomarkers into the search window, via the Multiple Proteins Homo sapiens analysis. (FIG. 8). FIG. 8 shows the network analysis for nominally significant predictive biomarkers for low mood/depression/hospitalizations across all subjects in the independent test cohort (n=23 genes, 26 probe sets).

CFG beyond Mood: evidence for involvement in other psychiatric and related disorders. A CFG approach was also used to examine evidence from other psychiatric and related disorders, as exemplified for the list of biomarkers after Step 4 testing (Table 7). A gene name that is not underlined indicates an increase in expression (I) in High Mood whereas an underlined gene names denotes a decrease in expression in High Mood (D).

### Step 4: Testing for Clinical Utility in Independent Cohorts

Independent cohorts of psychiatric patients were tested for the ability of each of the mood disorder biomarkers (n=26) to assess state severity (mood (SASS), depression (HAMD), mania (YMRS)), and predict trait risk (future hospitalizations for depression, future hospitalizations for mania). The analyses were conducted across all patients, as well as personalized by gender and diagnosis.

The test cohort for predicting low mood/depression (state), and the test cohort for predicting future Hospitalizations with Depression (trait), were assembled out of data that was RMA normalized by gender and diagnosis. The cohort was completely independent from the discovery and validation cohorts; there was no subject overlap with them. Individual markers used for predictions were Z scored by gender and diagnosis, to be able to combine different markers into panels and to avoid potential artifacts due to different ranges of expression in different gender and diagnoses. For biomarker panels, biomarkers were combined by simple summation of the increased risk biomarkers minus the decreased risk biomarkers. Predictions were performed using RStudio (RStudio is a free, open source IDE for R). For cross-sectional analyses, biomarker expression levels were used, and z-scored by gender and diagnosis. For the longitudinal analyses, four measures were combined: marker expression levels, slope (defined as the ratio of levels at current testing visit vs. previous visit, divided by the elapsed time between visits), maximum levels (at any of the current or past visits), and maximum slope (between any adjacent current or past visits). For decreased biomarkers markers, the minimum rather than the maximum for level calculations were used. All four measures were Z-scored, then combined in an additive fashion into a single measure. The longitudinal analysis was carried out in a sub-cohort of the testing cohort comprising of subjects having had at least two test visits.

Predicting State—Low Mood, Clinical Depression. Receiver-operating characteristic (ROC) analyses between marker levels and mood state were performed by assigning subjects visits with a mood SMS-7 score of ≤40 into the low mood category, and subjects with HAMD scores ≥22 in the clinically depressed category. The pROC package of R (Xavier Robin et al. BMC Bioinformatics 2011) was used. (Table 3, as applied in FIGS. 2A-2H). Additionally, a one-tailed t-test was performed between the low mood group versus the other groups, and Pearson R (one-tail) was calculated between mood scores and marker levels. Similar analyses were conducted for a high mood state (SMS-7 score of ≥60) and a clinical mania state (YMRS≥20).

Predicting Trait Future Psychiatric Hospitalization with Depression as a Symptom/Reason for Admission. Analyses for predicting future psychiatric hospitalizations with depression as a symptom/reason for admission in the first year following each testing visit were conducted in subjects that had at least one year of follow-up in the VA system, who also had a complete electronic medical record. ROC analyses between biomarkers measures (cross-sectional, longitudinal) at a specific testing visit and future hospitalization were performed as described above, based on assigning if a subject had been admitted to the hospital with depression or not with depression. Additionally, a one tailed t-test with unequal variance was performed between groups of subject visits with and without future hospitalization with depression. Pearson R (one-tail) correlation was performed on the data from the groups between hospitalization frequency (number of hospitalizations with depression divided by duration of follow-up) and marker levels. A Cox regression was performed using the time as measured in days from the testing visit date to first hospitalization date in the case of patients who had been hospitalized, or 365 days for those who did not. The hazard ratio was calculated such that a value greater than 1 always indicates increased risk for hospitalization, regardless if the biomarker is increased or decreased in expression.

Cox regression and Pearson R analyses were also conducted for all future hospitalizations with depression, including those occurring beyond one year of follow-up, in the years following testing (on average 5.12 years per subject, range 0.07 to 11.27 years), as these calculations, unlike the ROC and t-test, account for the actual length time elapsing until a follow-up visit, which varied from subject to subject. The ROC and t-test might under-represent the power of the biomarkers to predict, as the more severe psychiatric patients are more likely to move geographically and/or be lost to follow-up medical examination at the same hospital or facility. The Cox regression was performed using the time in days from visit date to first hospitalization date in the case of patients who had hospitalizations with depression, or from visit date to last note date in the electronic medical records for those who did not. Similar analyses were conducted for future hospitalizations with mania as a Symptom/Reason for Admission.

### Step 5: Therapeutics

### Pharmacogenomics

A CFG approach was used to examine evidence from other psychiatric and related disorders, for the list of the mood disorder biomarkers (for depression and mania after Steps 1-4, a total of n=13 genes, 14 probe sets) after Step 4 testing (Table 6). QIAGEN Ingenuity Pathway Analysis Software, was used to analyze individual mood disorder biomarkers and to determine which biomarkers were known to be modulated by existing drugs using the CFG databases results using this software are presented in Table 3 and Table 7).

Drugs and natural compounds were analyzed for compounds that exhibited an opposite match, i.e., compounds which altered the abnormal gene expression signatures of the depression biomarkers, as determined using the Connectivity Map (https://portals.broadinstitute.org, Broad Institute, MIT) (Tables 4A1-4B1) in a direction opposite to the one in depression. Not all the probe sets from the HG-U133 Plus 2.0 array were present in the HGU-133A array used for the Connectivity Map. Exact probe set level matches were used, not gene level imputation The NIH LINCS database was used to conduct similar analyses matching the gene expression signatures to drugs, at a biomarker gene level

Table 8 depicts sample pharmacogenomics and matching of the biomarkers for Low Mood/Depression (BioM12 Depression). Table 8 includes biomarkers that are targets of existing drugs and are modulated by them in opposite direction to depression/same direction as high mood. (I) means increased in expression, (D) means decreased in expression.

### Report Generation

The methods as disclosed herein may be used to generate a report for use by a medical professional. One aspect of such a report is shown in FIG. 4. The BioM 12 (the panel of n=12 genes, 13 probe sets), was used to generate a score for depression severity, as well as the mania biomarker RLP3 to inform risk for bipolar switching. Out of a dataset of 794 subject visits this report was generated as a case study based on a visit from a female patient with depression who had died by suicide, as previously described (Levey et al. 2016).

To generate the report shown in FIG. 4, a patient from the dataset, and the remaining dataset was distributed into 3 populations: those having a high HAMD score, those having an intermediate HAMD score, and those having a low HAMD score. For the three groups, the average Z-scored expression values were calculated for each biomarker in the panel. The levels of the biomarkers in each subject in the cohort were then compared, including the subject of interest, to these reference levels. If a biomarker was higher than the average of the high HAMD subjects, it received a score of 1. If the biomarker was below the average of the low HAMD subjects, the biomarker received a score of 0, and if the biomarker was in between the average of the high HAMD subjects and the average of the low HAMS subjects, then the biomarker received a score of 0.5.

TABLE 1
Demographics of cohorts used.

| | | | | | |
|---|---|---|---|---|---|
| Number of | | | | Age | T-test |
| Subjects | Gender | Diagnosis | Ethnicity | Mean (SD) | for age |

Discovery

| | | | | | |
|---|---|---|---|---|---|
| Discovery Cohort | 44 | Male = 30 | BP = 14 | EA = 33 | All = 50.76 |
| (Within-Subject | (with 134 | Female = 14 | MDD = 8 | AA = 9 | (6.48) |
| Changes in | visits) | | SZA = 5 | Asian = 1 | |
| mood VAS (SMS-7)) | | | SZ = 6 | Hispanic = 1 | |
| | | | PTSD = 8 | | |
| | | | MOOD = 2 | | |

Validation

| | | | | | |
|---|---|---|---|---|---|
| Independent Validation | 30 | Male = 23 | BP = 12 | EA = 27 | All = 49.42 |
| Cohort | | Female = 7 | MDD = 12 | AA = 2 | (7.06) |
| (Clinically Severe | | | SZA = 2 | Asian = 1 | |
| Depression) | | | PTSD = 3 | | |
| HAMD ≥ 22 | | | PSYCH = 1 | | |
| Independent Validation | 17 | Male = 16 | BP = 8 | EA = 11 | All = 48.25 |
| Cohort | | Female = 1 | SZA = 6 | AA = 6 | (8.21) |
| (Clinically Severe | | | SZ = 2 | | |
| Mania) | | | Psych = 1 | | |
| YMRS ≥ 20 | | | | | |

Testing

| | | | | | | |
|---|---|---|---|---|---|---|
| Independent Testing | 190 | Male = 153 | BP = 52 | EA = 118 | All = 50.52 | T-test for age |
| Cohort | | Female = 37 | MDD = 30 | AA = 69 | (8.58) | between Low |
| State Predictions- | | | SZA = 48 | Hispanic = 2 | Low Mood = 49 | Mood vs. High |
| Low mood | | | SZ = 36 | Mixed = 1 | (n = 87) | Mood and |
| (SMS-7 ≤ 40) | | | PTSD = 16 | | High Mood and | Intermediate |
| | | | Mood NOS = 5 | | Others = 50.88 | 0.104698 |
| | | | Psych NOS = 3 | | (n = 359) | |
| Independent Testing | 226 | Male = 181 | BP = 74 | EA = 156 | All= 46.71 | T-test for age |
| Cohort | | Female = 45 | MDD = 39 | AA = 66 | (9.42) | between Clinical |
| State Predictions | | | SZA = 48 | Asian = 1 | Clinical | Depression vs. |
| Clinical Depression- | | | SZ = 36 | Hispanic = 2 | Depression = | Others |
| (HAMD ≥ 22) | | | PTSD = 21 | Mixed = 1 | 44.4 (n = 40) | 0.177087984 |
| | | | Mood NOS = 5 | | Others = | |
| | | | Psych NOS = 3 | | 46.9 (n = 445) | |
| Independent Testing | 147 | Male = 130 | BP = 37 | EA = 90 | All = 47.13 | T-test for age |
| Cohort | | Female = 17 | MDD = 27 | AA = 54 | (9.38) | .Hosp with |
| Trait Predictions | | | SZA = 32 | Mixed = 1 | Others = | Depression vs. |
| Hospitalizations with | | | SZ = 33 | Hispanic = 2 | 47.23 (n = 282) | others |
| Depression | | | PTSD = 13 | | Hosp with | First year |
| First Year Following | | | MOOD = 3 | | Depression = | 0.701909278 |
| Initial Visit | | | PSYCH = 2 | | 46.58 (n = 50) | |
| Independent Testing | 170 | Male = 150 | BP = 41 | EA = 102 | All = 49.4 | T-test for age |
| Cohort | | Female = 20 | MDD = 29 | AA = 65 | (9.78) | Hosp with |
| Trait Predictions | | | SZA = 40 | Mixed = 1 | Others = | Depression vs. |
| Hospitalizations with | | | SZ = 39 | Hispanic = 2 | 49.4 (n = 282) | Others |
| Depression | | | PTSD = 14 | | Hosp with | All Future Years |
| All Future Years Following | | | MOOD = 5 | | Depression = | 0.93467396 |
| Initial Visit | | | PSYCH = 2 | | 49.3 (n = 127) | |
| Independent Testing | 190 | Male = 153 | BP = 52 | EA = 118 | All = 50.52 | T-test for age |
| Cohort | | Female = 37 | MDD = 30 | AA = 69 | (8.58) | between High |
| State Predictions - | | | SZA = 48 | Hispanic = 2 | High Mood = 50.6 | Mood vs. Low |
| High mood | | | SZ = 36 | Mixed = 1 | (n = 185) | and Intermediate |
| (SMS-7 ≥ 60) | | | PTSD = 16 | | Low Mood and | Mood |
| | | | Mood NOS = 5 | | Others = 50.5 | 0.877948 |
| | | | Psych NOS = 3 | | (n = 261) | |
| Independent Testing | 97 | Male = 73 | BP = 37 | EA = 72 | All = 39.4 | T-test for age |
| Cohort | | Female = 24 | MDD = 13 | AA = 22 | (8.83) | between Mania |
| State Predictions | | | SZA = 18 | Hispanic = 2 | Clinical Mania = | vs. Others |
| Clinical Mania - | | | SZ = 18 | Mixed = 1 | 38.9 (n = 13) | 0.883113775 |
| (YMRS ≥ 20) | | | PTSD = 10 | | Others = | |
| | | | Mood NOS = 1 | | 39.4 (n = 197) | |
| Independent Testing | 147 | Male = 130 | BP = 37 | EA = 90 | All = 47.13 | T-test for age |
| Cohort | | Female = 17 | MDD = 27 | AA = 54 | (9.38) | Hosp with no |
| Trait Predictions | | | SZA = 32 | Mixed = 1 | Hosp with no | Mania vs. Hosp |
| Hospitalizations (“Hosp”) | | | SZ = 33 | Hispanic = 2 | Mania = | with Mania |
| with Mania | | | PTSD = 13 | | 47.2 (n = 321) | within the first |
| First Year Following | | | MOOD = 3 | | Hosp with | Year |
| Initial Visit | | | PSYCH = 2 | | Mania = | 0.588179 |
| | | | | | 45.5 (n = 11) | |
| Independent Testing | 117 | Male = 102 | BP = 34 | EA = 74 | All = 44.39 | T-test for age |
| Cohort | | Female = 15 | MDD = 17 | AA = 40 | (9.01) | Hosp with no |
| Trait Predictions | | | SZA = 26 | Mixed = 1 | Hosp with no | Mania vs. Hosp |
| Hospitalizations | | | SZ = 26 | Hispanic = 2 | Mania= | with Mania |
| with Mania | | | PTSD = 11 | | 44.5 (n = 220) | within the |
| All Future | | | MOOD = 2 | | Hosp with | first Year |
| Years Following | | | PSYCH = 1 | | Mania = | 0.692290398 |
| Initial Visit | | | | | 43.7 (n = 37) | |

To generate the report, first, the biomarkers in the panel were averaged and multiplied by 100, yielding a score between 0 and 100 for the BioM12 for each of the 794 subjects, including the case study subject. This digitalization of the scores was performed to avoid overfitting the data to the particular cohort, and to provide readily understandable and interpretable readouts for clinicians. The score of the BioM12 was compared to the average score of BioM 12 for the high HAMD subjects and the low HAMD subjects, generating 3 risk categories which were indicated using a color scale of high (red), intermediate (yellow), and low (green) for current depression severity. The percentile of the score of the patient compared to the distribution of scores of subjects in the database was also provided in the report.

TABLE 2A
Biology of mood biomarkers: pathway analyses.

| | DAVID GO Functional Annotation | | | | | | | | | Ingenuity Pathways | | |
|---|---|---|---|---|---|---|---|---|---|---|---|---|
| | Biological Processes | | | | | KEGG Pathways | | | | Top | | |
| | | | | | P- | | | | P- | Canonical | P- | |
| | # | Term | Count | % | Value | Term | Count | % | Value | Pathways | Value | Overlap |
| Candidate | 1 | Regulation of | 9 | 39.1 | 5.20E−04 | Neurotrophin | 3 | 13 | 3.10E−02 | Serotonin | 8.62E−04 | 4.7% |
| biomarkers | | cell | | | | signaling | | | | Receptor | | 2/43 |
| (n = 26 | | differentiation | | | | pathway | | | | Signaling | | |
| probe | 2 | Rhythmic | 5 | 21.7 | 6.80E−04 | | | | | Glutamate | 1.51E−03 | 3.5% |
| sets, 23 | | process | | | | | | | | Receptor | | 2/57 |
| genes) | | | | | | | | | | Signaling | | |
| | 3 | Regulation of | 3 | 13 | 1.10E−03 | | | | | ErbB2- | 1.96E−03 | 3.1% |
| | | peptidy1- | | | | | | | | ErbB3 | | 2/65 |
| | | threonine | | | | | | | | Signaling | | |
| | | phosphorylation | | | | | | | | | | |
| | 4 | Mesenchymal | 4 | 17.4 | 1.30E−03 | | | | | Glutamine | 2.02E−03 | 50.0% |
| | | cell | | | | | | | | Degradation | | 1/2 |
| | | development | | | | | | | | I | | |
| | 5 | Circadian | 4 | 17.4 | 1.40E−03 | | | | | Cell Cycle: | 2.08E−03 | 3.0% |
| | | rhythm | | | | | | | | G1/S | | 2/67 |
| | | | | | | | | | | Checkpoint | | |
| | | | | | | | | | | Regulation | | |

TABLE 2B
Biology of mood biomarkers: diseases.

| | | | | | P- | Diseases and | P- | # |
|---|---|---|---|---|---|---|---|---|
| | # | Term | Count | % | Value | Disorders | Value | Molecules |
| Candidate | 1 | Weight Gain | 5 | 21.7 | 2.90E−05 | Neurological | 2.85E−03- | 18 |
| biomarkers | | | | | | Disease | 5.36E−09 | |
| (n = 26 | 2 | Major | 4 | 17.4 | 4.00E−05 | Psychological | 1.41E−03- | 14 |
| probe | | depressive | | | | Disorders | 1.14E−08 | |
| sets, 23 | | disorder | | | | | | |
| genes) | 3 | Schizophrenia | 8 | 34.8 | 5.10E−05 | Organismal Injury | 3.03E−03- | 23 |
| | | | | | | and Abnormalities | 1.91E−07 | |
| | 4 | Depression | 5 | 21.7 | 5.40E−05 | Skeletal and Muscular | 2.70E−03- | 11 |
| | | | | | | Disorders | 1.44E−06 | |
| | 5 | Psychosis | 3 | 13 | 1.60E−04 | Metabolic Disease | 2.02E−03- | 11 |
| | | | | | | | 1.51E−06 | |

Second, future risk for the subjects was assessed by looking at how many of the 3 identified biomarkers that are good predictors of future risk (i.e., NRG1, PRPS1, SMAD7) lie in the high-risk zone. In this system patients were based the presence of specific biomarkers patients were assigned between 0 to 3 asterisks.

TABLE 3A
Convergent Functional Evidence (CFE): biomarkers for low mood/depression,

| | | | | | Step 4 | |
|---|---|---|---|---|---|---|
| | | | | | Best | Step 4 |
| | | | | | Significant | Best |
| | | | | | Prediction | Significant |
| | | | | | of Low | Prediction of |
| | | | Step 2 | | Mood | Depression |
| | | | External | | State | State |
| | | Step 1 | Convergent | | ROC | ROC |
| | | Discovery | Functional | | AUC/ | AUC/ |
| | | in Blood | Genomics | | p-value | p-value |
| | | (Direction | (CFG) | Step 3 | 3 pts | 3 pts |
| | | of Change | Evidence For | Validation | All | All |
| | | in Low | Involvement | in Blood | 2 pts | 2 pts |
| Gene | | Mood) | in Mood | ANOVA | Gender | Gender |
| Symbol/ | | Method/ | Disorders | p-value/ | 1 pts | 1 pts |
| Gene | | Score/% | Score | Score | Gender/ | Gender/ |
| Name | Probesets | 6 pts | 12 pts | 6 pts | Dx | Dx |
| NRG1 | 208230_s_at | (I) | 10 | 2.80E−03/4 | All | Gender |
| Neuregulin 1 | | DE/2 | | Nominal | C: (87/446) | Females |
| | | 33.7% | | | 0.56/4.03E−02 | L: 2/49 |
| | | | | | L: (46/256) | 0.87/3.85E−02 |
| | | | | | 0.62/6.78E−03 | Gender/Dx |
| | | | | | Gender | M-PTSD |
| | | | | | Males | L: 3/10 |
| | | | | | C: (64/364) | 1/8.35E−03 |
| | | | | | 0.59/1.30E−02 | |
| | | | | | L: (37/211) | |
| | | | | | 0.62/1.29E−02 | |
| | | | | | Gender/Dx | |
| | | | | | M-MDD | |
| | | | | | L: (9/30) | |
| | | | | | 0.69/4.93E−02 | |
| DOCK10 | 219279_at | (D) | 10 | 4.95E−02/4 | Gender/Dx | All |
| dedicator of | | DE/2 | | Nominal | M-PSYCHOSIS | L: 15/259 |
| cytokinesis | | 41.5% | | | C: 31/182 | 0.73/1.17E−03 |
| 10 | | | | | 0.63/1.24E−02 | Gender |
| | | | | | M-SZA | Males |
| | | | | | C: 20/95 | L: 13/210 |
| | | | | | 0.7/2.92E−03 | 0.75/1.05E−03 |
| | | | | | L: 14/56 | Gender/Dx |
| | | | | | 0.65/4.79E−02 | M-PSYCHOSIS |
| | | | | | | L: 5/88 |
| | | | | | | 0.73/4.10E−02 |
| | | | | | | M-PTSD |
| | | | | | | L: 3/10 |
| | | | | | | 0.95/1.52E−02 |
| GLS | 203159_at | (D) | 8 | 1.90E−02/4 | Gender/Dx | All |
| glutaminase | | DE/4 | | Nominal | F-PTSD | L: 15/259 |
| | | 53.7% | | | C: 7/10 | 0.64/3.04E−02 |
| | | | | | 0.86/4.37E−02 | |
| | | | | | Gender/Dx | |
| | | | | | M-PSYCHOSIS | |
| | | | | | L: 20/110 | |
| | | | | | 0.63/3.43E−02 | |
| | | | | | Gender/Dx | |
| | | | | | M-SZA | |
| | | | | | C: 20/95 | |
| | | | | | 0.63/3.26E−02 | |
| | | | | | Gender/Dx | |
| | | | | | M-SZA | |
| | | | | | L: 14/56 | |
| | | | | | 0.72/7.72E−03 | |
| PRPS1 | 209440_at | (I) | 9 | 1.23E−03/4 | Gender/Dx | All |
| phosphoribosyl | | DE/4 | | Nominal | M-PSYCHOSIS | L: 15/259 |
| pyrophosphate | | 57.3% | | | C: 31/182 | 0.63/4.48E−02 |
| synthetase 1 | | | | | 0.63/1.05E−02 | Gender |
| | | | | | Gender/Dx | Males |
| | | | | | M-SZA | L: 13/210 |
| | | | | | C: 20/95 | 0.64/4.93E−02 |
| | | | | | 0.72/1.11E−03 | |
| TMEM161B | 227861_at | (I) | 10 | 7.11E−03/4 | Gender/Dx | All |
| transmembrane | | AP/4 | | Nominal | M-SZA | L: 15/259 |
| protein | | 62.1% | | | C: 20/95 | 0.63/4.48E−02 |
| 161B | | | | | 0.64/2.65E−02 | Gender |
| | | | | | | Males |
| | | | | | | L: 13/210 |
| | | | | | | 0.66/3.02E−02 |
| | | | | | | Gender/Dx |
| | | | | | | M-PTSD |
| | | | | | | L: 3/10 |
| | | | | | | 0.86/4.37E−02 |
| GLO1 | 200681_at | (I) | 12 | 2.11E−02/4 | Gender/Dx | Gender |
| glyoxalase I | | DE/2 | | Nominal | M-SZA | Males |
| | | 41.5% | | | C: 20/95 | L: 13/210 |
| | | | | | 0.66/1.33E−02 | 0.64/4.69E−02 |
| | | | | | Gender/Dx | Gender/Dx |
| | | | | | M-SZA | M-PTSD |
| | | | | | L: 14/56 | C: 7/24 |
| | | | | | 0.66/4.09E−02 | 0.72/4.62E−02 |
| FANCF | 218689_at | (I) | 8 | 3.46E−02/4 | Gender/Dx | All |
| Fanconi | | DE/4 | | Nominal | M-SZA | L: 15/259 |
| anemia | | 54.9% | | | C: 20/95 | 0.67/1.37E−02 |
| complementation | | | | | 0.64/3.13E−02 | Gender |
| group F | | | | | | Males |
| | | | | | | L: 13/210 |
| | | | | | | 0.66/2.57E−02 |
| HNRNPDL | 212454_x_at | (I) | 10 | 3.57E−02/4 | Gender/Dx | All |
| heterogeneous | | DE/2 | | Nominal | M-PSYCHOSIS | L: 15/259 |
| nuclear | | 35.4% | | | C: 31/182 | 0.63/4.97E−02 |
| ribonucleoprotein | | | | | 0.6/4.62E−02 | Gender |
| D like | | | | | Gender/Dx | Males |
| | | | | | M-PSYCHOSIS | L: 13/210 |
| | | | | | L: 20/110 | 0.65/3.39E−02 |
| | | | | | 0.64/2.78E−02 | |
| | | | | | Gender/Dx | |
| | | | | | M-SZA | |
| | | | | | C: 20/95 | |
| | | | | | 0.66/1.67E−02 | |
| | | | | | Gender/Dx | |
| | | | | | M-SZA | |
| | | | | | L: 14/56 | |
| | | | | | 0.67/2.80E−02 | |
| NRG1 | 208232_x_at | (D) | 10 | 3.78E−01/2 | Gender/Dx | |
| neuregulin 1 | | AP/4 | | Stepwise | F-PTSD | |
| | | 60.7% | | | C: 7/10 | |
| | | | | | 0.86/4.37E−02 | |
| | | | | | Gender/Dx | |
| | | | | | M-PSYCHOSIS | |
| | | | | | C: 31/182 | |
| | | | | | 0.6/4.14E−02 | |
| | | | | | Gender/Dx | |
| | | | | | M-SZA | |
| | | | | | C: 20/95 | |
| | | | | | 0.62/4.92E−02 | |
| | | | | | Gender/Dx | |
| | | | | | M-SZA | |
| | | | | | L: 14/56 | |
| | | | | | 0.65/4.43E−02 | |
| CD47 | 213856_at | (I) | 8 | 1.88E−02/4 | | All |
| CD47 | | AP/4 | | Nominal | | L: 15/259 |
| molecule | | 66.7% | | | | 0.68/9.55E−03 |
| | | | | | | Gender |
| | | | | | | Males |
| | | | | | | L: 13/210 |
| | | | | | | 0.73/3.30E−03 |
| | | | | | | Gender/Dx |
| | | | | | | M-MDD |
| | | | | | | L: 2/34 |
| | | | | | | 0.86/4.61E−02 |
| OLFM1 | 210924_at | (D) | 10 | 4.75E−02/4 | Gender/Dx | All |
| olfactomedin 1 | | DE/2 | | Nominal | M-PSYCHOSIS | L: 15/259 |
| | | 33.7% | | | C: 31/182 | 0.66/1.69E−02 |
| | | | | | 0.59/4.85E−02 | Gender |
| | | | | | Gender/Dx | Females |
| | | | | | M-SZA | L: 2/49 |
| | | | | | L: 14/56 | 0.85/4.77E−02 |
| | | | | | 0.68/2.45E−02 | Gender/Dx |
| | | | | | | M-PSYCHOSIS |
| | | | | | | C: 10/162 |
| | | | | | | 0.66/4.41E−02 |
| | | | | | | Gender/Dx |
| | | | | | | M-SZA |
| | | | | | | C: 7/84 |
| | | | | | | 0.7/4.09E−02 |
| SMAD7 | 204790_at | (I) | 9 | 4.57E−02/4 | Gender/Dx | All |
| SMAD | | DE/2 | | Nominal | F-BP | L: 15/259 |
| family | | 42.7% | | | L: 2/16 | 0.65/2.26E−02 |
| member 7 | | (I) | | | 0.93/2.84E−02 | Gender |
| | | AP/4 | | | Gender/Dx | Males |
| | | 54.0% | | | M-PSYCHOSIS | L: 13/210 |
| | | | | | C: 31/182 | 0.66/2.83E−02 |
| | | | | | 0.61/2.90E−02 | |
| | | | | | Gender/Dx | |
| | | | | | M-SZA | |
| | | | | | C: 20/95 | |
| | | | | | 0.64/3.13E−02 | |
| SLC6A4 | 242009_at | (D) | 10 | 5.28E−02/2 | Gender/Dx | All |
| solute | | DE/4 | | Stepwise | M-SZA | C: 40/485 |
| carrier | | 64.1% | | | L: 14/56 | 0.61/1.07E−02 |
| family 6 | | | | | 0.68/2.05E−02 | L: 15/259 |
| (neurotransmitter | | | | | | 0.66/1.78E−02 |
| transporter), | | | | | | Gender |
| member 4 | | | | | | Females |
| | | | | | | C: 5/94 |
| | | | | | | 0.78/1.80E−02 |
| | | | | | | Females |
| | | | | | | L: 2/49 |
| | | | | | | 0.98/1.15E−02 |
| | | | | | | Males |
| | | | | | | C: 35/391 |
| | | | | | | 0.59/3.93E−02 |
| | | | | | | Gender/Dx |
| | | | | | | M-PTSD |
| | | | | | | C: 7/24 |
| | | | | | | 0.72/4.61E−02 |

| | | Step 4 | | | |
|---|---|---|---|---|---|
| | | Best | | | |
| | | Significant | | | |
| | Step 4 | Predictions | | | |
| | Significant | of All | | | |
| | Prediction | Future | | | |
| | of First | Hosp for | | | |
| | Year Hosp | Depression | | | |
| | for | OR/OR | | | |
| | Depression | p-value | | | |
| | All | Updated | | | |
| | Gender | 1-tailed | | | |
| | Best in | p-value and | | | |
| | Individualized | added new | | | |
| | Gender/ | data for | | | |
| | Dx | genes that | | | |
| | ROC | has now | | | |
| | AUC/ | sigif | | Step 6 | |
| | p-value | p-value | | Drugs | |
| | 3 pts | 3 pts | | that | CFE |
| | All | All | Step 5 | Modulate the | Polyevidence |
| | 2 pts | 2 pts | Other | Biomarker | Score for |
| Gene | Gender | Gender | Psychiatric | in Same | Involvement |
| Symbol/ | 1 pts | 1 pts | and Related | Direction | in Depression |
| Gene | Gender/ | Gender/ | Disorders | as High | (Based on |
| Name | Dx | Dx | Evidence | Mood | Steps 1-4) |
| NRG1 | Gender | All | Aging | Antidepressants | 26 |
| Neuregulin 1 | Females | C: (127/409) | Anxiety | Antipsychotic | |
| | C: (7/41) | 1.17/2.51E−02 | Dementia | Antipsychotics | |
| | 0.87/1.15E−03 | Gender | Male-BP | Escitalopram | |
| | Gender/Dx | Females | Suicide | (SSRI) | |
| | F-MDD | C: (11/50) | Memory | Lithium | |
| | C: (3/7) | 1.59/4.99E−02 | MetaSuicide | | |
| | 1/1.69E−02 | Gender/Dx | Pain | | |
| | F-PTSD | M-PSYCHOSIS | Psychosis | | |
| | C: (2/11) | C: (62/184) | PTSD | | |
| | 1/1.69E−02 | 1.22/2.36E−02 | Stimulants | | |
| | M-PTSD | M-SZA | Stress | | |
| | C: (2/13) | C: (34/88) | Suicide | | |
| | 0.91/3.78E−02 | 1.34/2.99E−03 | SZ | | |
| DOCK10 | Gender | Gender | Aging | Ketamine | 24 |
| dedicator of | Females | Females | Alcohol | Physical and | |
| cytokinesis | C: 7/41 | C: 11/50 | BP | Cognitive | |
| 10 | 0.71/4.48E−02 | 1.9/3.93E−02 | Dementia | stimulation | |
| | Gender/Dx | | Female | | |
| | F-BP | | Suicide | | |
| | C: 2/13 | | MetaSuicide | | |
| | 0.91/3.78E−02 | | Social | | |
| | F-PTSD | | Defeat | | |
| | C: 2/11 | | Stress | | |
| | 0.94/2.97E−02 | | SZ | | |
| GLS | Gender | Gender | Aging | Clozapine | 24 |
| glutaminase | Females | Females | Alcohol | Omega-3 fatty | |
| | C: 7/41 | C: 11/50 | Anxiety | acids | |
| | 0.82/4.23E−03 | 2.25/9.70E−03 | ASD | Risperidone | |
| | Gender/Dx | Gender/Dx | Dementia | | |
| | F-BP | F-BP | Female | | |
| | C: 2/13 | C: 4/13 | Suicide | | |
| | 0.95/2.42E−02 | 6.25/2.93E−02 | MetaSuicide | | |
| | | | Pain | | |
| | | | PTSD | | |
| | | | Stress | | |
| | | | Suicide | | |
| | | | SZ | | |
| PRPS1 | Gender/Dx | Gender | Aging | Lithium | 24 |
| phosphoribosyl | F-PTSD | Females | ASD | | |
| pyrophosphate | C: 2/11 | C: 11/50 | Dementia | | |
| synthetase 1 | 0.94/2.97E−02 | 1.85/3.28E−02 | Female | | |
| | | Gender/Dx | Suicide | | |
| | | M-SZA | Male Suicide | | |
| | | C: 34/88 | MetaSuicide | | |
| | | 1.41/1.94E−02 | PTSD | | |
| | | | Stress | | |
| | | | SZ | | |
| TMEM161B | Gender | | Alcohol | | 24 |
| transmembrane | Females | | ASD | | |
| protein | C: 7/41 | | Female | | |
| 161B | 0.79/8.41E−03 | | Suicide | | |
| | Gender/Dx | | Male-BP | | |
| | F-BP | | Suicide | | |
| | C: 2/13 | | MetaSuicide | | |
| | 0.91/3.78E−02 | | Neurological | | |
| | Gender/Dx | | Sleep | | |
| | F-PTSD | | Stress | | |
| | C: 2/11 | | | | |
| | 0.89/4.95E−02 | | | | |
| GLO1 | Gender/Dx | Gender/Dx | Anxiety | Omega-3 | 22.5 |
| glyoxalase I | F-BP | F-BP | ASD | fatty | |
| | C: 2/13 | C: 4/13 | Dementia | acids | |
| | 0.91/3.78E−02 | 3.32/4.97E−02 | MetaSuicide | | |
| | | | Panic | | |
| | | | Sleep | | |
| | | | Stress | | |
| | | | SZ | | |
| FANCF | Gender | | Stress | | 22 |
| Fanconi | Females | | | | |
| anemia | C: 7/41 | | | | |
| complementation | 0.72/3.58E−02 | | | | |
| group F | Gender/Dx | | | | |
| | F-BP | | | | |
| | C: 2/13 | | | | |
| | 1/1.50E−02 | | | | |
| | Gender/Dx | | | | |
| | F-PTSD | | | | |
| | C: 2/11 | | | | |
| | 0.89/4.95E−02 | | | | |
| HNRNPDL | Gender/Dx | Gender/Dx | Aging | Benzodiazepines | 22 |
| heterogeneous | F-BP | F-BP | Anxiety | Omega-3 | |
| nuclear | C: 2/13 | C: 4/13 | ASD | fatty | |
| ribonucleoprotein | 0.95/2.42E−02 | 3.83/4.89E−02 | Stress | acids | |
| D like | | Gender/Dx | Dementia | | |
| | | M-SZA | Female | | |
| | | C: 34/88 | Suicide | | |
| | | 1.39/3.19E−02 | Hallucinogens | | |
| | | | Male Suicide | | |
| | | | MetaSuicide | | |
| | | | Mood | | |
| | | | Stabilizers | | |
| | | | PTSD | | |
| | | | Stress | | |
| | | | SZ | | |
| NRG1 | Gender/Dx | Gender/Dx | Aging | Antidepressants | 22 |
| neuregulin 1 | F-PTSD | M-SZA | Anxiety | Antipsychotic | |
| | C: 2/11 | C: 34/88 | Behavior | Antipsychotics | |
| | 1/1.69E−02 | 1.36/1.86E−02 | Dementia | Escitalopram | |
| | | | Male-BP | (SSRI) | |
| | | | Suicide | Lithium | |
| | | | Memory | | |
| | | | MetaSuicide | | |
| | | | Pain | | |
| | | | Psychosis | | |
| | | | PTSD | | |
| | | | Stimulants | | |
| | | | Stress | | |
| | | | Suicide | | |
| | | | SZ | | |
| CD47 | Gender | | Aging | Clozapine | 21 |
| CD47 | Females | | Stress | Lithium | |
| molecule | C: 7/41 | | Dementia | Omega-3 | |
| | 0.71/4.48E−02 | | Female | fatty | |
| | Gender/Dx | | Suicide | acids | |
| | F-BP | | Male Suicide | Venlafaxine | |
| | C: 2/13 | | MetaSuicide | | |
| | 0.91/3.78E−02 | | Pain | | |
| | | | Sleep | | |
| | | | Stress | | |
| | | | SZ | | |
| OLFM1 | Gender/Dx | | Aging | Valproate | 21 |
| olfactomedin 1 | F-PTSD | | Alcohol | | |
| | C: 2/11 | | Hallucinogens | | |
| | 0.89/4.95E−02 | | PTSD | | |
| | | | Stress | | |
| | | | Suicide | | |
| | | | SZ | | |
| SMAD7 | | | Aging | Antidepressants | 21 |
| SMAD | | | Anxiety | | |
| family | | | Dementia | | |
| member 7 | | | Female | | |
| | | | Suicide | | |
| | | | Stress | | |
| | | | SZ | | |
| SLC6A4 | | | Aging | Antidepressants | 20 |
| solute | | | Alcohol | Exposure | |
| carrier | | | Antipsychotics | therapy | |
| family 6 | | | Anxiety | Lithium | |
| (neurotransmitter | | | ASD | Omega-3 | |
| transporter), | | | Female | fatty | |
| member 4 | | | Suicide | acids | |
| | | | Hallucinogens | Remifentanil | |
| | | | Male-BP | | |
| | | | Suicide | | |
| | | | MetaSuicide | | |
| | | | OCD | | |
| | | | Pain | | |
| | | | Panic | | |
| | | | Personality | | |
| | | | PTSD | | |
| | | | Stress | | |
| | | | Suicide | | |
| | | | SZ | | |

TABLE 3B
Convergent Functional Evidence (CFE), biomarkers for bipolar mood disorders.

| | | | | | Step 4 | | Step 4 |
|---|---|---|---|---|---|---|---|
| | | | | | Best | Step 4 | Significant |
| | | | | | Significant | Best | Prediction |
| | | | | | Prediction | Significant | of First |
| | | | Step 2 | | of Low | Prediction | Year |
| | | | External | | Mood | of Depression | Hosp for |
| | | Step 1 | Convergent | | State | State | Depression |
| | | Discovery | Functional | | ROC | ROC | ROC |
| | | in Blood | Genomics | | AUC/ | AUC/ | AUC/ |
| | | (Direction | (CFG) | Step 3 | p-value | p-value | p-value |
| | | of Change | Evidence | Validation | 3 pts | 3 pts | 3 pts |
| | | in High | For | in Blood | All | All | All |
| | | Mood) | Involvement | ANOVA | 2 pts | 2 pts | 2 pts |
| Genesymbol/ | Probe | Method/ | in Mood | p-value/ | Gender | Gender | Gender |
| Gene | Set | Score/% | Score | Score | 1 pts | 1 pts | 1 pts |
| name | ID | 6 pts. | 12 pts. | 6 pts. | Gender/Dx | Gender/Dx | Gender/Dx |
| NRG1 | 208230_s_at | (D) | 10.00 | 2.80E−03/4 | ALL | Gender | Gender |
| Neuregulin 1 | | DE/2 | | Nominal | C: (87/446) | Females | Females |
| | | 33.7% | | | 0.56/4.03E−02 | L: (2/49) | C: (7/41) |
| | | | | | L: (46/256) | 0.87/3.85E−02 | 0.87/1.15E−03 |
| | | | | | 0.62/6.78E−03 | Gender/Dx | Gender/Dx |
| | | | | | Gender | M-PTSD | F-MDD |
| | | | | | Males | L: (3/10) | C: (3/7) |
| | | | | | C: (64/364) | 1/8.35E−03 | 1/1.69E−02 |
| | | | | | 0.59/1.30E−02 | | F-PTSD |
| | | | | | L: (37/211) | | C: (2/11) |
| | | | | | 0.62/1.29E−02 | | 1/1.69E−02 |
| | | | | | Gender/Dx | | M-PTSD |
| | | | | | M-MDD | | (2/13) |
| | | | | | L: (9/30) | | 0.91/3.78E−02 |
| | | | | | 0.69/4.93E−02 | | |
| DOCK10 | 219279_at | (I) | 10.00 | 4.95E−02/4 | Gender/Dx | ALL | Gender |
| Dedicator Of | | DE/2 | | Nominal | M-PSYCHOSIS | L: | Females |
| Cytokinesis 10 | | 41.5% | | | C: (31/182) | (15/259) | C: (7/41) |
| | | | | | 0.63/1.24E−02 | 0.73/1.17E−03 | 0.71/4.48E−02 |
| | | | | | M-SZA | Gender | Gender/Dx |
| | | | | | C: (20/95) | Males | F-BP |
| | | | | | 0.7/2.92E−03 | L: | C: (2/13) |
| | | | | | L: (14/56) | (13/210) | 0.91/3.78E−02 |
| | | | | | 0.65/4.79E−02 | 0.75/1.05E−03 | F-PTSD |
| | | | | | | Gender/Dx | C: (2/11) |
| | | | | | | M-PSYCHOSIS | 0.94/2.97E−02 |
| | | | | | | L: (5/88) | |
| | | | | | | 0.73/4.10E−02 | |
| | | | | | | M-PTSD | |
| | | | | | | L: (3/10) | |
| | | | | | | 0.95/1.52E−02 | |
| GLS | 203159_at | (I) | 8.00 | 1.90E−02/4 | Gender/Dx | ALL | Gender |
| Glutaminase | | DE/4 | | Nominal | F-PTSD | L: (15/259) | Females |
| | | 53.7% | | | C: (7/10) | 0.64/3.04E−02 | C: (7/41) |
| | | | | | 0.86/4.37E−02 | | 0.82/4.23E−03 |
| | | | | | M-PSYCHOSIS | | Gender/Dx |
| | | | | | L: (20/110) | | F-BP |
| | | | | | 0.63/3.43E−02 | | C: (2/13) |
| | | | | | Gender/Dx | | 0.95/2.42E−02 |
| | | | | | M-SZA | | |
| | | | | | C: (20/95) | | |
| | | | | | 0.63/3.26E−02 | | |
| | | | | | L: (14/56) | | |
| | | | | | 0.72/7.72E−03 | | |
| PRPS1 | 209440_at | (I) | 9.00 | 1.23E−03/4 | Gender/Dx | ALL | Gender/Dx |
| Phosphoribosyl | | DE/4 | | Nominal | M-PSYCHOSIS | L: (15/259) | F-PTSD |
| Pyrophosphate | | 57.3% | | | C: (31/182) | 0.63/4.48E−02 | C: (2/11) |
| Synthetase 1 | | | | | 0.63/1.05E−02 | Gender | 0.94/2.97E−02 |
| | | | | | M-SZA | Males | |
| | | | | | C: (20/95) | L: (13/210) | |
| | | | | | 0.72/1.11E−03 | 0.64/4.93E−02 | |
| TMEM161B | 227861_at | (I) | 10.00 | 7.11E−03/4 | Gender/Dx | ALL | Gender |
| Transmembrane | | AP/4 | | Nominal | M-SZA | L: (15/259) | Females |
| Protein | | 62.1% | | | C: (20/95) | 0.63/4.48E−02 | C: (7/41) |
| 161B | | | | | 0.64/2.65E−02 | Gender | 0.79/8.41E−03 |
| | | | | | | Males | Gender/Dx |
| | | | | | | L: (13/210) | F-BP |
| | | | | | | 0.66/3.02E−02 | C: (2/13) |
| | | | | | | Gender/Dx | 0.91/3.78E−02 |
| | | | | | | M-PTSD | F-PTSD |
| | | | | | | L: (3/10) | C: (2/11) |
| | | | | | | 0.86/4.37E−02 | 0.89/4.95E−02 |
| SLC6A4 | 242009_at | (D) | 10.00 | 5.28E−02/2 | Gender/Dx | ALL | |
| Solute | | DE/4 | | Stepwise | M-SZA | C: (40/485) | |
| Carrier | | 64.1% | | | L: (14/56) | 0.61/1.07E−02 | |
| Family 6 | | | | | 0.68/2.05E−02 | L: (15/259) | |
| Member 4 | | | | | | 0.66/1.78E−02 | |
| | | | | | | Gender | |
| | | | | | | Females | |
| | | | | | | C: (5/94) | |
| | | | | | | 0.78/1.80E−02 | |
| | | | | | | L: | |
| | | | | | | (2/49) | |
| | | | | | | 0.98/1.15E−02 | |
| | | | | | | Gender | |
| | | | | | | Males | |
| | | | | | | C: (35/391) | |
| | | | | | | 0.59/3.93E−02 | |
| | | | | | | Gender/Dx | |
| | | | | | | M-PTSD | |
| | | | | | | C: (7/24) | |
| | | | | | | 0.72/4.61E−02 | |

| | | | | | Step 4 | | |
|---|---|---|---|---|---|---|---|
| | Step 4 | Step 4 | | Step 4 | Best | | |
| | Best | Best | Step 4 | Significant | Significant | | |
| | Significant | Significant | Best | Prediction | Predictions | | |
| | Predictions | Prediction | Significant | of First | of All | | |
| | of ALL | of High | Prediction | Year | Future | | |
| | Future | Mood | of Mania | Hosp for | Hosp for | | |
| | Hosp for | State | State | Mania | Mania | | |
| | Depression | ROC | ROC | ROC | Cox | Drugs | |
| | Cox OR/ | AUC/ | AUC/ | AUC/ | OR/ | that | |
| | p-value | p-value | p-value | p-value | p-value | Modulate | |
| | 3 pts | 3 pts | 3 pts | 3 pts | 3 pts | the | |
| | All | All | All | All | All | Biomarker | CFE |
| | 2 pts | 2 pts | 2 pts | 2 pts | 2 pts | in Same | Poly- |
| Genesymbol/ | Gender | Gender | Gender | Gender | Gender | Direction | evi- |
| Gene | 1 pts | 1 pts | 1 pts | 1 pts | 1 pts | as High | dence |
| name | Gender/Dx | Gender/Dx | Gender/Dx | Gender/Dx | Gender/Dx | Mood | Score |
| NRG1 | ALL | All | | | Gender/Dx | Mood | 30 |
| Neuregulin 1 | C: (127/409) | L: (109/254) | | | M-PSYCHOSIS | Stabilizers | |
| | 1.17/2.51E−02 | 0.58/1.39E−02 | | | L: (7/55) | Antidepressants | |
| | Gender | Gender | | | 2.67/3.27E−02 | Antipsychotics | |
| | Females | Males | | | M-SZ | | |
| | C: (11/50) | L: (99/209) | | | L: (4/31) | | |
| | 1.59/4.99E−02 | 0.59/1.38E−02 | | | 3.76/3.54E−02 | | |
| | Gender/Dx | | | | | | |
| | M-PSYCHOSIS | | | | | | |
| | C: (62/184) | | | | | | |
| | 1.22/2.36E−02 | | | | | | |
| | M-SZA | | | | | | |
| | C: (34/88) | | | | | | |
| | 1.34/2.99E−03 | | | | | | |
| DOCK10 | Gender | Gender | | | | Physical | 26 |
| Dedicator Of | Females | Females | | | | and | |
| Cytokinesis 10 | C: (11/50) | L: (10/45) | | | | Cognitive | |
| | 1.9/3.93E−02 | 0.70/2.63E−02 | | | | stimulation | |
| | | Gender/Dx | | | | | |
| | | F-BP | | | | | |
| | | C: (9/30) | | | | | |
| | | 0.73/2.45E−02 | | | | | |
| | | F-BP | | | | | |
| | | L: (5/16) | | | | | |
| | | 1.0/9.18E−04 | | | | | |
| GLS | Gender | Gender | | | | Omega-3 | 26 |
| Glutaminase | Females | Females | | | | fatty | |
| | C: (11/50) | C: (19/82) | | | | acids | |
| | 2.25/9.70E−03 | 0.64/3.20E−02 | | | | Antipsychotics | |
| | Gender/Dx | Gender/Dx | | | | | |
| | F-BP | F-BP | | | | | |
| | C: (4/13) | C: (9/30) | | | | | |
| | 6.25/2.93E−02 | 0.79/5.28E−03 | | | | | |
| | | L: (5/16) | | | | | |
| | | 0.85/1.36E-02 | | | | | |
| | | M-Psychosis | | | | | |
| | | L: (48/110) | | | | | |
| | | 0.61/2.27E−02 | | | | | |
| | | M-SZ | | | | | |
| | | L: (24/54) | | | | | |
| | | 0.72/2.98E−03 | | | | | |
| PRPS1 | Gender | Gender: | | | | | 26 |
| Phosphoribosyl | Females | Females | | | | | |
| Pyrophosphate | C: (11/50) | C: (19/82) | | | | | |
| Synthetase 1 | 1.85/3.28E−02 | 0.64/3.45E−02 | | | | | |
| | Gender/Dx | L: (10/45) | | | | | |
| | M-SZA | 0.74/1.02E−02 | | | | | |
| | C: (34/88) | Gender/Dx | | | | | |
| | 1.41/1.94E−02 | F-BP | | | | | |
| | | C: (9/30) | | | | | |
| | | 0.75/1.58E−02 | | | | | |
| | | L: (5/16) | | | | | |
| | | 0.96/1.93E−03 | | | | | |
| TMEM161B | | Gender/Dx | | | | | 25 |
| Transmembrane | | F-BP | | | | | |
| Protein | | C: (9/30) | | | | | |
| 161B | | 0.69/4.93E−02 | | | | | |
| | | L: (5/16) | | | | | |
| | | 0.82/2.37E−02 | | | | | |
| SLC6A4 | | Gender/Dx | Gender/Dx | All: | | Remifentanil | 25 |
| Solute | | F-BP | M-Psychosis | C: (11/332) | | Omega-3 | |
| Carrier | | C: (9/30) | L: (1/27) | 0.74/3.33E−03 | | fatty | |
| Family 6 | | 0.73/2.45E−02 | 1/4.76E−02 | Gender: | | acids | |
| Member 4 | | L: (5/16) | | Males | | Mood | |
| | | 0.85/1.36E−02 | | C: (10/291) | | Stabilizers | |
| | | | | 0.72/8.33E−03 | | Antidepressants | |
| | | | | Gender/Dx | | | |
| | | | | M-BP | | | |
| | | | | C: (6/71) | | | |
| | | | | 0.77/1.35E−02 | | | |
| | | | | M-MDD | | | |
| | | | | C: (1/55) | | | |
| | | | | 1/4.45E−02 | | | |

TABLE 3C
CFE. Convergent Functional Evidence (CFE), biomarkers for high mood/mania.

| | | | | | Step 4 | |
|---|---|---|---|---|---|---|
| | | | | | Best | Step 4 |
| | | | | | Significant | Best |
| | | | | | Prediction | Significant |
| | | | Step 2 | | of High | Prediction |
| | | | External | | Mood | of Mania |
| | | | Convergent | | State | State |
| | | Step 1 | Functional | | ROC | ROC |
| | | Discovery | Genomics | | AUC/ | AUC/ |
| | | in Blood | (CFG) | Step 3 | p-value | p-value |
| | | (Direction of | Evidence | Validation | 3 pts | 3 pts |
| | | Change in | For | in Blood | All | All |
| Gene | | High Mood) | Involvement | ANOVA | 2 pts | 2 pts |
| Symbol/ | | Method/ | in Mood | p-value/ | Gender | Gender |
| Gene | | Score/% | Score | Score | 1 pts | 1 pts |
| Name | Probesets | 6 pt | 12 pts | 6 pts | Gender/Dx | Gender/Dx |
| RPL3 | 212039_x_at | (I) | 8 | 3.32E−02/4 | Gender | |
| Ribosomal | | DE/4 | | Nominal | Females | |
| Protein | | 50% | | | C: (19/82) | |
| L3 | | | | | 0.66/1.86E−02 | |
| | | | | | Gender/Dx | |
| | | | | | F-BP | |
| | | | | | C: (9/30) | |
| | | | | | 0.82/3.54E−03 | |
| | | | | | L: (5/16) | |
| | | | | | 0.85/1.36E−02 | |
| SLC6A4 | 242009_at | (D) | 10 | 5.28E−02/2 | Gender/Dx | Gender/Dx |
| solute | | DE/4 | | Stepwise | F-BP | M-Psychosis |
| carrier | | 64.1% | | | C: (9/30) | L: (1/27) |
| family 6 | | | | | 0.73/2.45E−02 | 1/4.76E−02 |
| (neurotransmitter | | | | | L: (5/16) | |
| transporter), | | | | | 0.85/1.36E−02 | |
| member 4 | | | | | | |

| | | Step 4 | | | |
|---|---|---|---|---|---|
| | | Best | | | |
| | | Significant | | | |
| | | Predictions | | | |
| | | of All | | | |
| | | Future | | | |
| | Step 4 | Hosp for | | | |
| | Significant | Mania | | | |
| | Prediction | OR/OR | | | |
| | of First | p-value | | | |
| | Year | Updated | | | |
| | Hosp for | 1-tailed | | | |
| | Mania | p-value | | | |
| | All | and | | | |
| | Gender | added | | | |
| | Best in | new data | | | |
| | Individualized | for genes | | | |
| | Gender/Dx | that has | | | |
| | ROC | now | | Step 6 | |
| | AUC/ | sigif | | Drugs | |
| | p-value | p-value | | that | CFE |
| | 3 pts | 3 pts | Step 5 | Modulate | Polyevidence |
| | All | All | Other | the | Score for |
| Gene | 2 pts | 2 pts | Psychiatric | Biomarker in | Involvement |
| Symbol/ | Gender | Gender | and Related | OppositeDirection | in Mania |
| Gene | 1 pts | 1 pts | Disorders | as High | (Based on |
| Name | Gender/Dx | Gender/Dx | Evidence | Mood | Steps 1-4) |
| RPL3 | All: | | | anisomycin | 21 |
| Ribosomal | C: (11/332) | | | | |
| Protein | 0.68/2.18E−02 | | | | |
| L3 | Gender: | | | | |
| | Males | | | | |
| | C: (10/291) | | | | |
| | 0.66/3.99E−02 | | | | |
| SLC6A4 | All: | Gender/Dx | | | 21 |
| solute | C: (11/332) | F-BP | | | |
| carrier | 0.74/3.33E−03 | C: (9/30) | | | |
| family 6 | Gender: | 0.73/2.45E−02 | | | |
| (neurotransmitter | Males | L: (5/16) | | | |
| transporter), | C: (10/291) | 0.85/1.36E−02 | | | |
| member 4 | 0.72/8.33E−03 | | | | |
| | Gender/Dx | | | | |
| | M-BP | | | | |
| | C: (6/71) | | | | |
| | 0.77/1.35E−02 | | | | |
| | M-MDD | | | | |
| | C: (1/55) | | | | |
| | 1/4.45E−02 | | | | |

Third, the number of the bipolar biomarkers (n=6) in the panel was examined, to see how many biomarkers had a value of 1. If more than 50% of the biomarkers were present in an abnormal fashion (more than 3 out of 6), the patient received an asterisk (or like indicator) for bipolar risk. If the mania biomarker RLP3 also had a score of 1 then the patient received a second asterisk (or like indicator) for risk of bipolarity, i.e. risk of switch if treated for depression. In those with a high risk (a represented in this example by 3 asterisks), it was available to choose mood stabilizers or antipsychotics from the medication choices provided by the report.

TABLE 4A1
Therapeutics: Drug repurposing for depression. Connectivity Map (CMAP) analyses
drugs identified using gene expression panels of biomarkers with highest evidence
(CFE) for involvement in depression (BioM12 Depression 12 genes, 13 probests).
See Table 3A. Direction of expression in high mood. (out of 13 probesets, 8 increased
and 3 decreased probesets were present in HG-U133A array used by CMAP).

| rank | cmap name | score | Role |
|---|---|---|---|
| 1 | isoflupredone | 1 | Synthetic glucocorticoid that may be considered as an |
| | | | alternative to traditional corticosteroids. Isoflupredone |
| | | | is the only corticosteroid approved by the U.S. Food |
| | | | and Drug Administration for use exclusively in large |
| | | | animals, including lactating cattle. |
| 2 | trichostatin A | 0.963 | HDAC inhibitor |
| 3 | dubinidine | 0.943 | Anticonvulsant which reduces motor activity, |
| | | | enhances the effects of alcohol, ether and barbiturates. |
| | | | Quinoline alkaloid, from plants of the Rutaceae |
| | | | Family. |
| 4 | ciprofibrate | 0.939 | PPAR-alpha activator, lipid lowering agent |
| 5 | pioglitazone | 0.931 | PPAR-γ activator, anti-diabetic |
| 6 | tropine | 0.93 | Alkaloid |
| 7 | adiphenine | 0.907 | Anticholinergic, antispasmodic |
| 8 | saquinavir | 0.903 | Anti-retroviral medication |
| 9 | amitriptyline | 0.902 | Tricyclic anti-depressant. |
| 10 | chlorogenic acid | 0.897 | Antioxidant, polyphenol found in coffee |

TABLE 4A2
Therapeutics: Drug repurposing, drugs identified using gene expression panels
of biomarkers with highest evidence (CFE) for involvement in depression without
overlap with bipolar (BioM6 Depression Specific - 6 genes, 7 probestes). Direction
of expression in high mood. (Out of 7 probesets, 5 increased and 2 decreased
biomarkers were present in HG-U133A array used by CMAP).

| rank | cmap name | score | Role |
|---|---|---|---|
| 1 | pindolol | 1 | β-blocker, and is also a potent serotonin |
| | | | 5HT_{1A} presynaptic receptor antagonist |
| 2 | lansoprazole | 0.977 | Proton pump inhibitor (PPI), that works by |
| | | | decreasing the amount of acid produced by the |
| | | | stomach. |
| 3 | xamoterol | 0.975 | Cardiac stimulant, that works by binding to the β₁ |
| | | | adrenergic receptor. It is a 3rd generation adrenergic |
| | | | β receptor partial agonist. It provides cardiac |
| | | | stimulation at rest but it acts as a blocker during |
| | | | exercise. |
| 4 | methanthelinium | 0.953 | Muscarinic receptor antagonist (anticholinergic, |
| | bromide | | parasympatholytic agent). Spasmolytic agent. Gastric |
| | | | acid secretion inhibitor. |
| 5 | asiaticoside | 0.927 | Triterpenoid component derived from Centella |
| | | | asiatica (L.) and widely used in antioxidant, |
| | | | antiinflammatory, immunomodulatory, and wound |
| | | | healing applications. |
| 6 | estradiol | 0.924 | Female sex hormone |
| 7 | methacoline | 0.923 | Muscarinic agonist |
| 8 | isoflupredone | 0.916 | Steroid |
| 9 | carteolol | 0.913 | Beta-blocker |
| 10 | chlorcyclizine | 0.911 | First-generation antihistamine. It is used primarily to |
| | | | treat allergy symptoms such as rhinitis, urticaria, and |
| | | | pruritus, and may also be used as an antiemetic. |

TABLE 4A3
Therapeutics: Drug repurposing, drugs identified using gene expression panels of
biomarkers overlapping between depression and bipolar (BioM6 Bipolar - 6 genes,
6 probesets). Direction of expression in high mood. (Out of 6 probesets, 4 increased
and 1 decreased probesets were present in HG-U133A array used by CMAP).

| rank | cmap name | score | Role |
|---|---|---|---|
| 1 | valproic acid | 1 | HDAC inhibitor, mood stabilizer |
| 2 | atracurium besilate | 0.991 | Nicotinic antagonist muscle relaxant |
| 3 | Chicago Sky Blue | 0.98 | Histological stain that also is a vesicular glutamate |
| | 6B | | transporters inhibitor, attenuating methamphetamine- |
| | | | induced hyperactivity and behavioral sensitization in |
| | | | animal models |
| 4 | enoxacin | 0.972 | Fluoroquinolone antibiotic that also elevates |
| | | | microRNA levels and prevents learned helplessness in |
| | | | animal models |
| 5 | levobunolol | 0.969 | Beta-blocker |
| 6 | 15-delta | 0.95 | Anti-inflammatory lipid mediator and PPAR-γ |
| | prostaglandin J2 | | activator. It is made from prostaglandin D2. Decreased |
| | | | Prostaglandin D2 Levels in Major Depressive |
| | | | Disorder Are Associated with Depression-Like |
| | | | Behaviors in human and animal model studies. |
| 7 | ciprofibrate | 0.949 | PPAR-alpha activator, lipid lowering agent |
| 8 | pirinixic acid | 0.949 | PPAR-alpha activator, anti-lipid agent |
| 9 | isoflupredone | 0.947 | Synthetic glucocorticoid |
| 10 | trichostatin A | 0.946 | HDAC inhibitor |

TABLE 4B1
NIH LINCS drugs identified using gene expression panels of biomarkers
with Highest Evidence (CFE) for involvement in depression (BioM12
Depression - 12 genes). See Table 3A. Direction of expression
in high mood (9 increased and 4 decreased).

| Rank | Score | Drug | Description |
|---|---|---|---|
| 1 | 0.3 | NNC 55-0396 | T-type calcium channel |
| | | dihydrochloride | blocker |
| 2 | 0.3 | Nadolol | Beta blocker |
| 3 | 0.3 | MLN4924 | Inhibitor of Nedd8- |
| | | | Activating Enzyme |
| 4 | 0.2 | U0126 | MEK ½ inhibitor |
| 5 | 0.2 | Nortryptiline | Tricyclic |
| | | | antidepressant |
| 6 | 0.2 | Amcinonide | Synthetic |
| | | | glucocorticoid |
| 7 | 0.2 | Iopanic acid | Iodine-containing |
| | | | radiocontrast medium, |
| | | | thyroid inhibitor |
| 8 | 0.2 | Paroxetine | SSRI antidepressant |
| 9 | 0.2 | Rosuvastatin | Statin |
| 10 | 0.2 | trichostatin A | HDAC inhibitor |

Fourth, for each biomarker in the panel, a list of existing psychiatric medications that modulate the expression of the biomarker in the direction of high mood was referenced. Each medication received a score commensurate with the biomarker score, i.e. 1 or 0.5 or 0. A medication can modulate more than one biomarker. An average score for each medication was calculated based on its effects on the biomarkers in the panel, and multiplied by 100, resulting in a score of 0 to 100 for each medication. Thus, psychiatric medications were matched to the patient and ranked in order of impact on the panel.

TABLE 5
23 Mood disorder biomarkers (after first 3 steps).

| | | Direction of |
|---|---|---|
| | | change in |
| Gene Symbol | Gene Name | high mood |
| NRG1 | Neuregulin 1 | D |
| TMEM161B | Transmembrane Protein 161B | I |
| PRPS1 | Phosphoribosyl Pyrophosphate | I |
| | Synthetase 1 | |
| GLS | Glutaminase | I |
| DOCK10 | Dedicator Of Cytokinesis 10 | I |
| GLO1 | Glyoxalase I | I |
| HNRNPDL | Heterogeneous Nuclear | I |
| | Ribonucleoprotein D Like | |
| FANCF | Fanconi Anemia Complementation | I |
| | Group F | |
| SMAD7 | SMAD Family Member 7 | I |
| CD47 | CD47 Molecule | I |
| OLFM1 | Olfactomedin 1 | D |
| CALM1 | Calmodulin 1 | I |
| SPECC1 | Sperm Antigen With Calponin | D |
| | Homology And Coiled-Coil | |
| | Domains 1 | |
| ANK3 | Ankyrin 3 | I |
| OGT | O-Linked N-Acetylglucosamine | I |
| | (GlcNAc) Transferase | |
| RPL3 | Ribosomal Protein L3 | I |
| TPH1 | Tryptophan Hydroxylase 1 | D |
| MARCKS | Myristoylated Alanine Rich Protein | D |
| | Kinase C Substrate | |
| TMEM106B | Transmembrane Protein 106B | I |
| SORT1 | Sortilin 1 | D |
| GSK3B | Glycogen Synthase Kinase 3 Beta | D |
| NR3C1 | Nuclear Receptor Subfamily 3 | D |
| | Group C Member 1 | |
| SLC6A4 | Solute Carrier Family 6 Member 4 | D |

Fifth, the biomarkers that were positive as high risk in the panel were used to interrogate the CMAP for individualized drug repurposing, identifying new non-psychiatric compounds that could be used in a particular patient to treat depression.

TABLE 6
CFG for mood- used in step 2, prioritization for biomarkers
for low mood/depression (BioM12 depression) and mania (RLP3).

| | | | | Prior | |
|---|---|---|---|---|---|
| Gene | | (Direction | Prior | Human | Prior |
| Symbol/ | | of Change) | Human | Brain | Human |
| Gene | | Method/ | Genetic | Tissue | Peripheral |
| Name | Probesets | Score/% | Evidence | Evidence | Evidence |
| NRG1 | 208230_s_at | (D) | MDD (Hall, | (D) | (I) |
| neuregulin 1 | | DE/2 | Adams et al. | CA3/2 Stratum | PBMC MDD |
| | | 33.7% | 2018) | oriens BP | (Belzeaux, |
| | | | BP (Yu, | (Benes, Lim et | Formisano- |
| | | | Bi et al. | al. 2009) | Treziny et al. |
| | | | 2014), | (D) | 2010) |
| | | | (Walker, | Hippocampus | (D) |
| | | | Christoforou | BP (Benes, | PBMC |
| | | | et al. | Lim et al. 2009), | Antidepressants |
| | | | 2010), (Goes, | (Marballi, Cruz | (Belzeaux, |
| | | | Willour et al. | et al. 2012) | Formisano- |
| | | | 2009), (Gutierrez- | (D) | Treziny et al. |
| | | | Fernandez, | prefrontal | 2010) |
| | | | Palomino et | cortices MDD | (I) |
| | | | al. 2014), | (Tochigi, | peripheral |
| | | | (Thomson, | Iwamoto et al. | blood |
| | | | Christoforou | 2008) | mononuclear |
| | | | et al. 2007), | (D) | cells BP |
| | | | (Biernacka, | BA8/9 Female | (Begemann, |
| | | | Geske et al.) | MDD (Labonte, | Sargin et al. |
| | | | | Engmann et al. | 2008) |
| | | | | 2017) | |
| | | | | (D) | |
| | | | | Hippocampus | |
| | | | | BP (Marballi, | |
| | | | | Cruz et al. 2012) | |
| DOCK10 | 219279_at | (I) | BP | (I) | (I) |
| dedicator of | | DE/2 | (Kataoka, | Ventral | Blood |
| cytokinesis 10 | | 41.5% | Matoba et al. | Subiculum | BP (Beech, |
| | | | 2016) | Female MDD | Lowthert et |
| | | | Linkage | (Labonte, | al. 2010) |
| | | | | Engmann et al. | |
| | | | | 2017) | |
| GLS | 203159_at | (I) | | (I) BA46 MDD | (D) |
| Glutaminase | | DE/4 | | (Sequeira, | Lymphoblastoid |
| | | 53.7% | | Mamdani et al. | Cell |
| | | | | 2009) | Lines |
| | | | | (D) Brain BP | BP, MDD |
| | | | | (Chen, Wang et | (Martin, |
| | | | | al. 2013) | Rollins et al. |
| | | | | FPC | 2009) |
| | | | | differentially | |
| | | | | expressed genes | |
| | | | | MDD (Zhurov, | |
| | | | | Stead et al. 2012) | |
| | | | | (D) FTPFC | |
| | | | | MDD(Zhurov, | |
| | | | | Stead et al. 2012) | |
| | | | | (I)Anterior PFC | |
| | | | | BP (Gottschalk, | |
| | | | | Wesseling et al. | |
| | | | | 2014) | |
| PRPS1 | 209440_at | (I) | Linkage | (D) AMY MDD | Whole blood |
| Phosphoribosyl | | DE/4 | | (Forero, Guio- | DNA |
| Pyrophosphate | | 57.3% | | Vega et al. 2017) | Differentially |
| Synthetase 1 | | | | (D) BA 10 | methylated |
| | | | | MDD (Malki, | BP |
| | | | | Pain et al. 2015) | (Dempster, |
| | | | | Brain | Pidsley et al. |
| | | | | (D) DLPFC BP | 2011) |
| | | | | (Chen, Wang et | |
| | | | | al. 2013) | |
| | | | | (D) MDD | |
| | | | | (Kang, Adams et | |
| | | | | al. 2007) | |
| | | | | (D) PFC | |
| | | | | MDD(Malki, | |
| | | | | Pain et al. 2015) | |
| | | | | (I) PFC MDD | |
| | | | | (Forero, Guio- | |
| | | | | Vega et al. 2017) | |
| TMEM161B | 227861_at | (I) | Depression | (D) BA8/9 Male | (I) L neurons |
| Transmembrane | | AP/4 | (Howard, | MDD (Labonte, | BP (Kim, |
| Protein 161B | | 62.1% | Adams et al. | Engmann et al. | Liu et al. |
| | | | 2019) | 2017) | 2015) |
| | | | Depression | | |
| | | | (Hyde, Nagle | | |
| | | | et al. 2016) | | |
| GLO1 | 200681_at | (I) | MDD (Hall, | (D) Brain | (D) |
| Glyoxalase | | DE/2 | Adams et al. | BP (Chen, | Peripheral |
| I | | 41.5% | 2018) | Wang et al. | white blood |
| | | | | 2013) | cells BP |
| | | | | (D) | (Sun 1989) |
| | | | | Hippocampus | |
| | | | | BP (Benes, | |
| | | | | Matzilevich et al. | |
| | | | | 2006) | |
| | | | | (I) BA11, | |
| | | | | Subic Female | |
| | | | | MDD (Labonte, | |
| | | | | Engmann et al. | |
| | | | | 2017) | |
| | | | | (I) Anterior | |
| | | | | PFC BP, MDD | |
| | | | | (Gottschalk, | |
| | | | | Wesseling et al. | |
| | | | | 2014) | |
| FANCF | 218689_at | (I) | | (D) Brain | (D) Blood |
| Fanconi | | DE/4 | | BP (Chen, | MDD |
| Anemia | | 54.9% | | Wang et al. | (Forero, |
| Complementation | | | | 2013) | Guio-Vega et |
| Group F | | | | (I) BA11, | al. 2017) |
| | | | | BA25, Subic | |
| | | | | Female MDD | |
| | | | | (Labonte, | |
| | | | | Engmann et al. | |
| | | | | 2017) | |
| HNRNPDL | 212454_x_at | (I) | | (D) AMY and | (I) Fibroblast |
| Heterogeneous | | DE/2 | | cingulate cortex | MDD |
| Nuclear | | 35.4% | | MDD (Gaiteri, | (Garbett, |
| Ribonucleoprotein | | | | Guilloux et al. | Vereczkei et |
| D Like | | | | 2010) | al. 2015) |
| | | | | (I) NAC Male | |
| | | | | MDD (Labonte, | |
| | | | | Engmann et al. | |
| | | | | 2017) | |
| NRG1 | 208232_x_at | (D) | MDD (Hall, | (D) | (I) |
| Neuregulin 1 | | AP/4 | Adams et al. | CA3/2 Stratum | PBMC MDD |
| | | 60.7% | 2018) | oriens BP | (Belzeaux, |
| | | | BP (Yu, | (Benes, Lim et | Formisano- |
| | | | Bi et al. | al. 2009) | Treziny et al. |
| | | | 2014), | (D) | 2010) |
| | | | (Walker, | Hippocampus | (D) |
| | | | Christoforou | BP (Benes, | PBMC |
| | | | et al. | Lim et al. 2009), | Antidepressants |
| | | | 2010), (Goes, | (Marballi, Cruz | (Belzeaux, |
| | | | Willour et al. | et al. 2012) | Formisano- |
| | | | 2009), | (D) | Treziny et al. |
| | | | (Gutierrez- | prefrontal | 2010) |
| | | | Fernandez, | cortices MDD | (I) |
| | | | Palomino et | (Tochigi, | peripheral |
| | | | al. 2014), | Iwamoto et al. | blood |
| | | | (Thomson, | 2008) | mononuclear |
| | | | Christoforou | (D) | cells BP |
| | | | et al. 2007), | BA8/9 Female | (Begemann, |
| | | | (Takami, | MDD (Labonte, | Sargin et al. |
| | | | Higashi et | Engmann et al. | 2008) |
| | | | al.) | 2017) | (D) |
| | | | | (D) | peripheral |
| | | | | Hippocampus | blood |
| | | | | BP (Marballi, | mononuclear |
| | | | | Cruz et al. 2012) | cells MDD |
| | | | | | (Belzeaux, |
| | | | | | Formisano- |
| | | | | | Treziny et al. |
| | | | | | 2010) |
| CD47 | 213856_at | (I) | | (I) AMY | (D) |
| CD47 | | AP/4 | | and cingulate | Peripheral |
| Molecule | | 66.7% | | cortex MDD | venous blood |
| | | | | (Gaiteri, | MDD |
| | | | | Guilloux et al. | (Jansen, |
| | | | | 2010) | Penninx et |
| | | | | | al. 2016) |
| OLFM1 | 210924_at | (D) | BP | (D) Brain | (D) |
| Olfactomedin 1 | | DE/2 | (Johnson, | BP (Chen, | NT2.D1 cells |
| | | 33.7% | Drgon et al. | Wang et al. | Valproate |
| | | | 2009) | 2013) | (Hill, Nagel |
| | | | | (D) Cerebral | et al. 2013) |
| | | | | cortex BP | |
| | | | | (Gandal, Haney | |
| | | | | et al. 2018) | |
| SMAD7 | 204790_at | (I) | | (I) DLPFC | (D) Blood |
| SMAD | | DE/2 | | (BA46) BP | MDD |
| Family | | 42.7% | | (Nakatani, | (Mamdani, |
| Member 7 | | (I) | | Hattori et al. | Berlim et al. |
| | | AP/4 | | 2006) | 2014) |
| | | 54% | | (D) Anterior | (I) Blood |
| | | | | Insula Male | Antidepressants |
| | | | | MDD (Labonte, | (Hennings, |
| | | | | Engmann et al. | Uhr et al. |
| | | | | 2017) | 2015) |
| SLC6A4 | 242009_at | (D) | Affective | (D) | (D) |
| solute | | DE/4 | Disorder | BP (Kato and | MDD |
| carrier | | 64.1% | (Collier, | Iwamoto 2014) | (Watanabe, |
| family 6 | | | Stober et al. | (D) | Iga et al. |
| (neurotransmitter | | | 1996), (Lasky- | PFC | 2015) |
| transporter), | | | Su, Faraone | MDD (Mann, | (I) |
| member 4 | | | et al. 2005) | Huang et al. | PBMC |
| | | | BP | 2000) | Antidepressants |
| | | | (Rotondo, | (I) | (Belzeaux, |
| | | | Mazzanti et | Ventral | Formisano- |
| | | | al. 2002), | Subiculum | Treziny et al. |
| | | | (Neves, | Female MDD | 2010) |
| | | | Silveira et al. | (Labonte, | (D) |
| | | | 2008), (Wang, | Engmann et al. | Neural |
| | | | Lee et al. | 2017) | progenitor |
| | | | 2014), (Neves, | (D) | cells (NPCs) |
| | | | Malloy- | midbrain, | Antidepressants |
| | | | Diniz et al. | caudate BP | (Lopez, |
| | | | 2010), (Furlong, | (Hsu, Lirng et al. | Lim et al. |
| | | | Ho et al. | 2014) | 2014) |
| | | | 1998) | (D) | (I) |
| | | | Depression | Thalamus MDD | placenta |
| | | | (Luykx, | (Ho, Ho et al. | MDD |
| | | | Bakker et al. | 2013) | (Ponder, |
| | | | 2013) | | Salisbury et |
| | | | MDD | | al. 2011) |
| | | | (Ho, Ho et | | (I) |
| | | | al. | | PBMC cells |
| | | | 2013), | | MDD |
| | | | (Lopez-Leon, | | (Belzeaux, |
| | | | Janssens et | | Formisano- |
| | | | al. 2008), | | Treziny et al. |
| | | | (Caspi, | | 2010) |
| | | | Sugden et al. | | (I) |
| | | | 2003), (Lopez | | Blood |
| | | | de Lara, | | MDD(Belzeaux, |
| | | | Dumais et al. | | Azorin |
| | | | 2006), (Kilpa | | et al. 2014) |
| | | | trick, | | |
| | | | Koenen et al. | | |
| | | | 2007), (Muglia, | | |
| | | | Tozzi et | | |
| | | | al. | | |
| | | | 2010), (Lewis, | | |
| | | | Ng et al. | | |
| | | | 2010) | | |
| | | | Mood | | |
| | | | Disorders | | |
| | | | NOS | | |
| | | | (Brezo, | | |
| | | | Bureau et al. | | |
| | | | 2010) | | |
| | | | Lithium | | |
| | | | (Rybakowski, | | |
| | | | Czerski et | | |
| | | | al. 2012) | | |
| | | | Linkage | | |

Biomarker for Mania

| | | | | |
|---|---|---|---|---|
| RPL3 | 212039_x_at | (I) | (I) | (I) |
| Ribosomal | | DE/4 | BA11, (D) NAC | Blood |
| Protein | | 50% | Female MDD | MDD |
| L3 | | | (Labonte, | (Cordova- |
| | | | Engmann et al. | Palomera, |
| | | | 2017) | Fatjo-Vilas |
| | | | (I) | et al. 2015) |
| | | | BA25, BA8/9 | |
| | | | Male MDD | |
| | | | (Labonte, | |
| | | | Engmann et al. | |
| | | | 2017) | |

| | | Prior | | Step 2 | |
|---|---|---|---|---|---|
| | Prior | Non- | Prior | Prioritization | |
| Gene | Non- | human | Non- | CFG | |
| Symbol/ | human | Brain | human | Score | Step |
| Gene | Genetic | Tissue | Peripheral | For | 1-4 |
| Name | Evidence | Evidence | Evidence | Mood | CFE |
| NRG1 | | (I) | | 10 | 26 |
| neuregulin 1 | | AMY | | | |
| | | MDD | | | |
| | | (Surget, | | | |
| | | Wang et | | | |
| | | al. 2009), | | | |
| | | (Andrus, | | | |
| | | Blizinsky | | | |
| | | et al. | | | |
| | | 2012) | | | |
| DOCK10 | | (D) | | 10 | 24 |
| dedicator of | | PFC | | | |
| cytokinesis 10 | | MDD | | | |
| | | (Bagot, | | | |
| | | Cates et | | | |
| | | al. 2016) | | | |
| GLS | | (D) | | 8 | 24 |
| Glutaminase | | Hippocampus | | | |
| | | MDD(Andrus, | | | |
| | | Blizinsky | | | |
| | | et al. | | | |
| | | 2012) | | | |
| | | (I) AMY | | | |
| | | (males) | | | |
| | | BP (Le- | | | |
| | | Niculescu, | | | |
| | | McFarland | | | |
| | | et al. | | | |
| | | 2008) | | | |
| PRPS1 | | (I) PFC | | 9 | 24 |
| Phosphoribosyl | | MDD | | | |
| Pyrophosphate | | (Malki, | | | |
| Synthetase 1 | | Pain et al. | | | |
| | | 2015) | | | |
| TMEM161B | | (I) AMY | | 10 | 24 |
| Transmembrane | | MDD | | | |
| Protein 161B | | (Andrus, | | | |
| | | Blizinsky | | | |
| | | et al. | | | |
| | | 2012) | | | |
| GLO1 | Depression | (D) | (D) | 11.50 | 22.5 |
| Glyoxalase | related | mPFC | Hippocampus, | | |
| I | (McMurray, | Hippocampus, | | | |
| | Ramaker | Antidepressants | mPFC | | |
| | et al. | (McMurray, | Antidepressants | | |
| | 2018) | Ramaker | (McMurray, | | |
| | | et al. | Ramaker | | |
| | | 2018) | et al. | | |
| | | (D) | 2018) | | |
| | | Cortex | | | |
| | | Fluoxetine | | | |
| | | (Benton, | | | |
| | | Miller et | | | |
| | | al. 2012) | | | |
| FANCF | | (D) | | 8 | 22 |
| Fanconi | | AMY, PFC | | | |
| Anemia | | (males) | | | |
| Complementation | | BP (Le- | | | |
| Group F | | Niculescu, | | | |
| | | McFarland | | | |
| | | et al. | | | |
| | | 2008) | | | |
| HNRNPDL | | (D) | (D) | 10 | 22 |
| Heterogeneous | | Cerebral | Lymphocytes | | |
| Nuclear | | Cortex | (males) | | |
| Ribonucleoprotein | | (right) | BP (Le- | | |
| D Like | | Lithium | Niculescu, | | |
| | | (McQuillin, | McFarland | | |
| | | Rizig et | et al. | | |
| | | al. 2007) | 2008) | | |
| | | (I) AMY | | | |
| | | (males) | | | |
| | | BP (Le- | | | |
| | | Niculescu, | | | |
| | | McFarland | | | |
| | | et al. | | | |
| | | 2008) | | | |
| NRG1 | | (I) | | 10 | 22 |
| Neuregulin 1 | | AMY | | | |
| | | MDD | | | |
| | | (Surget, | | | |
| | | Wang et | | | |
| | | al. 2009), | | | |
| | | (Andrus, | | | |
| | | Blizinsky | | | |
| | | et al. | | | |
| | | 2012) | | | |
| CD47 | | (D) | | 8 | 21 |
| CD47 | | Hippocampus | | | |
| Molecule | | MDD | | | |
| | | (Zubenko, | | | |
| | | Hughes et | | | |
| | | al. 2014) | | | |
| OLFM1 | | (D) NAC | (D) Blood | 10.00 | 21 |
| Olfactomedin 1 | | MDD | MDD | | |
| | | (Bagot, | (Pajer, | | |
| | | Cates et | Andrus et | | |
| | | al. 2016) | al. 2012) | | |
| SMAD7 | | (D) | | 9 | 21 |
| SMAD | | Dentate | | | |
| Family | | Gyrus | | | |
| Member 7 | | Antidepressants, | | | |
| | | Fluoxetine | | | |
| | | (Surget, | | | |
| | | Wang et | | | |
| | | al. 2009) | | | |
| SLC6A4 | | (I) | | 10 | 20 |
| solute | | PFC | | | |
| carrier | | MDD | | | |
| family 6 | | (Park, | | | |
| (neurotransmitter | | Yoo et al. | | | |
| transporter), | | 2012) | | | |
| member 4 | | (I) | | | |
| | | Embryonic | | | |
| | | hippocampal, | | | |
| | | PFC | | | |
| | | neurons | | | |
| | | MDD | | | |
| | | (Hoyo- | | | |
| | | Becerra, | | | |
| | | Huebener | | | |
| | | et al. | | | |
| | | 2013) | | | |
| | | (I) Cortex | | | |
| | | Fluoxetine | | | |
| | | (Benton, | | | |
| | | Miller et | | | |
| | | al. 2012) | | | |
| Biomarker for Mania | | | | | |
| RPL3 | | (D) | | 8 | 21 |
| Ribosomal | | Ventral | | | |
| Protein | | Hippocampus | | | |
| L3 | | MDD | | | |
| | | 27181059 | | | |
| | | (I) PFC | | | |
| | | MDD, | | | |
| | | Ventral | | | |
| | | medial | | | |
| | | hippocampus | | | |
| | | (Bagot, | | | |
| | | Cates et | | | |
| | | al. 2016) | | | |

No underlining—increased in expression (I) in high mood; underlining—decreased in expression in High Mood (D); DE—differential expression; AP—Absent/Present.

We also created and used a checklist/measure of clinical severity of bipolar disorder, based on past history, called Convergent Functional Information for Bipolar Disorder Severity (CFI-BP) scale, ranking patients with mood disorders on a scale of 1 to 10. This is more a trait measure, related to how people behaved in their past.

TABLE 7
Evidence for involvement in other sdisorders for biomarkers for low mood/depression (BioM12 depression).

| | | (Direction | | Prior | |
|---|---|---|---|---|---|
| Gene | | of Change) | Prior | Human | Prior |
| Symbol/ | | Method/ | Human | Brain | Human |
| Gene | | Score/ | Genetic | Tissue | Peripheral |
| Name | Probesets | % | Evidence | Evidence | Evidence |
| DOCK10 | 219279_at | (I) | Aging (Erikson, | (D) Cerebellum | (D) |
| dedicator of | | DE/2 | Bodian et al. | Aging (Peters, | LymphocyteSZ |
| cytokinesis 10 | | 41.5% | 2016) | Joehanes et al. 2015) | {Bowden, 2006 |
| | | | | (D) Superior frontal | #723} |
| | | | | cortex Alcohol (Liu, | (D) Blood |
| | | | | Lewohl et al. 2006) | Aging (Peters, |
| | | | | (D) Parietal Lobe | Joehanes et al. |
| | | | | Dementia (Patel, | 2015) |
| | | | | Dobson et al. 2019) | (D) Blood |
| | | | | (D) OFC | Female Suicide |
| | | | | PTSD {Girgenti, | (Levey, Niculescu |
| | | | | 2020 #70517} | et al. 2016) |
| | | | | | (D) Blood |
| | | | | | MetaSuicide |
| | | | | | (Niculescu, Le- |
| | | | | | Niculescu et al. |
| | | | | | 2017) |
| | | | | | (D) Blood |
| | | | | | Stress (Le- |
| | | | | | Niculescu, |
| | | | | | Roseberry et al. |
| | | | | | 2019) |
| GLS | 203159_at | (I) | Aging (Harris, | (D) Cerebral cortex | (D) Blood |
| Glutaminase | | DE/4 | Riggio et al. | ASD, SZ (Gandal, | Female Suicide |
| | | 53.7% | 2017) | Haney et al. 2018) | (Levey, Niculescu |
| | | | SZ (Gulsuner, | (D) Temporal Cortex | et al. 2016) |
| | | | Walsh et al. 2013) | Dementia (Castillo, | (D) Blood |
| | | | SZ (Gandal, | Leon et al. 2017) | MetaSuicide |
| | | | Haney et al. 2018) | (D) Hippocampus | (Niculescu, Le- |
| | | | Suicide (Coon, | Dementia (Blalock, | Niculescu et al. |
| | | | Darlington et al. | Geddes et al. 2004) | 2017) |
| | | | 2018) | (D) BA46 | (D) Blood |
| | | | | Suicide (Sequeira, | Pain (Niculescu, |
| | | | | Mamdani et al. 2009) | Le-Niculescu et |
| | | | | (D) Frontopolar | al. 2019) |
| | | | | cortex Suicide | (D) PBMC |
| | | | | (Zhurov, Stead et al. | PTSD (Segman, |
| | | | | 2012) | Shefi et al. 2005) |
| | | | | (D) AMY | (D) Blood |
| | | | | SZ (Chang, Liu et | Memory |
| | | | | al. 2017) | retention |
| | | | | (D) DLPFC SZ | {Niculescu, 2019 |
| | | | | (Martin, Rollins et al. | #48328} |
| | | | | 2009) | |
| | | | | (D) Amygdala SZ | |
| | | | | (Chang, Liu et al. | |
| | | | | 2017) | |
| | | | | (D) Prefrontal | |
| | | | | Cortex SZ | |
| | | | | (Hagihara, Ohira et al. | |
| | | | | 2014) | |
| | | | | (D) DLPFC, | |
| | | | | Hippocampus, | |
| | | | | Associative striatum | |
| | | | | SZ (Lanz, | |
| | | | | Reinhart et al. 2019) | |
| PRPS1 | 209440_at | (I) | | (D) Forebrain neural | (D) PBMCs |
| Phosphoribosyl | | DE/4 | | progenitor cells | ER Trauma |
| Pyrophosphate | | 57.3% | | SZ (Roussos, | survivors |
| Synthetase 1 | | | | Guennewig et al. | (Segman, Shefi et |
| | | | | 2016) | al. 2005) |
| | | | | (D) Frontal and | (D) |
| | | | | temporal cortex, | Blood |
| | | | | Cerebral cortex | Aging (Peters, |
| | | | | Autism, SZ | Joehanes et al. |
| | | | | (Parikshak, Swarup et | 2015) |
| | | | | al. 2016, Gandal, | (D) Blood |
| | | | | Haney et al. 2018) | High Stress State |
| | | | | (D) Hippocampus | (Le-Niculescu, |
| | | | | Alzheimer's Disease | Roseberry et al. |
| | | | | (Blalock, Geddes et | 2019) |
| | | | | al. 2004) | (D) Blood |
| | | | | (D) Hippocampus, | Female Suicide |
| | | | | Associative striatum, | (Levey, Niculescu |
| | | | | SZ (Lanz, | et al. 2016) |
| | | | | Reinhart et al. 2019) | (D) Blood |
| | | | | | Male Suicide |
| | | | | | (Niculescu, Levey |
| | | | | | et al. 2015) |
| | | | | | (D) Blood |
| | | | | | MetaSuicide |
| | | | | | (Niculescu, Le- |
| | | | | | Niculescu et al. |
| | | | | | 2017) |
| TMEM161B | 227861_at | (I) | Alcohol (Muench, | | (D) Blood |
| Transmembrane | | AP/4 | Schwandt et al. | | Female Suicide |
| Protein | | 62.1% | 2018) | | (Levey, Niculescu |
| 161B | | | Sleep (Jansen, | | et al. 2016) |
| | | | Watanabe et al. | | (D) Blood |
| | | | 2019) | | MetaSuicide |
| | | | ASD, | | (Niculescu, Le- |
| | | | Neurological | | Niculescu et al. |
| | | | (Nowakowska, | | 2017) |
| | | | Obersztyn et al. | | (D) Blood |
| | | | 2010) | | Stress (Le- |
| | | | | | Niculescu, |
| | | | | | Roseberry et al. |
| | | | | | 2019) |
| | | | | | (D) Blood |
| | | | | | Memory |
| | | | | | retention |
| | | | | | {Niculescu, 2019 |
| | | | | | #48328} |
| GLO1 | 200681_at | (I) | Anxiety (Donner, | (D) Frontal cortex | (D) Blood |
| Glyoxalase I | | DE/2 | Pirkola et al. | ASD (Junaid, Kowal | MetaSuicide |
| | | 41.5% | 2008) | et al. 2004) | (Niculescu, Le- |
| | | | Sleep (Jansen, | (D) Hippocampus | Niculescu et al. |
| | | | Watanabe et al. | Dementia (Blalock, | 2017) |
| | | | 2019) | Geddes et al. 2004) | (D) |
| | | | Panic (Politi, | (D) Parietal Lobe | Lymphoblastoid |
| | | | Minoretti et al. | Dementia (Patel, | cell lines (LCLs) |
| | | | 2006) | Dobson et al. 2019) | SZ (Sanders, |
| | | | | (D) Hippocampus | Goring et al. |
| | | | | SZ (Lanz, Reinhart | 2013) |
| | | | | et al. 2019) | (D) plasma |
| | | | | (D) Dorsolateral | Aging (Lehallier, |
| | | | | prefrontal cortex | Gate et al. 2019) |
| | | | | SZ (Bowen, Burgess | |
| | | | | et al. 2019) | |
| | | | | (I) Anterior PFC | |
| | | | | SZ, SZA (Gottschalk, | |
| | | | | Wesseling et al. 2014) | |
| | | | | (D) Hippocampus | |
| | | | | Alzheimer's Disease | |
| | | | | {Zahid, 2014 | |
| | | | | #20060} | |
| FANCF | 218689_at | (I) | | | (D) Blood |
| Fanconi | | DE/4 | | | Stress (Le- |
| Anemia | | 54.9% | | | Niculescu, |
| Complementation | | | | | Roseberry et al. |
| Group F | | | | | 2019) |
| HNRNPDL | 212454_x_at | (I) | | (D) Cerebral cortex | (D) |
| Heterogeneous | | DE/2 | | ASD (Gandal, Haney | PBMCs Aging |
| Nuclear | | 35.4% | | et al. 2018) | (Harris, Riggio |
| Ribonucleoprotein | | | | (D) Hippocampus | et al. 2017) |
| D Like | | | | Dementia (Blalock, | (D) Blood |
| | | | | Geddes et al. 2004) | Aging (Peters, |
| | | | | (D) Parietal Lobe | Joehanes et al. |
| | | | | Dementia (Patel, | 2015) |
| | | | | Dobson et al. 2019) | (D) Blood |
| | | | | (I) PFC | Female Suicide |
| | | | | Suicide (Kekesi, | (Levey, Niculescu |
| | | | | Juhasz et al. 2012) | et al. 2016) |
| | | | | | (D) Blood |
| | | | | | Male Suicide |
| | | | | | (Niculescu, Levey |
| | | | | | et al. 2015) |
| | | | | | (D) Blood |
| | | | | | MetaSuicide |
| | | | | | (Niculescu, Le- |
| | | | | | Niculescu et al. |
| | | | | | 2017) |
| | | | | | (D) Blood |
| | | | | | PTSD (Breen, |
| | | | | | Tylee et al. 2018) |
| | | | | | (D) Blood |
| | | | | | Stress (Le- |
| | | | | | Niculescu, |
| | | | | | Roseberry et al. |
| | | | | | 2019) |
| | | | | | (Differentially |
| | | | | | methylated) Whole |
| | | | | | blood DNA SZ |
| | | | | | (Dempster, |
| | | | | | Pidsley et al. |
| | | | | | 2011) |
| NRG1 | 208232_x_at | (D) | Stimulants | (I) BA-9 PFC | (Differential |
| Neuregulin 1 | 208230_s_at | AP/4 | (Uhl, Drgon et al. | SZ (Marballi, Cruz | methylation) |
| | | 60.7% | 2008) | et al. 2012) | Blood PTSD |
| | | (D) | Aging (Levine | (I) CA3/2 | (I) Aging |
| | | DE/2 | and Crimmins | Stratum oriens | (Peters, Joehanes |
| | | 33.7% | 2016) | SZ (Benes, Lim et | et al. 2015) |
| | | | Anxiety (Dina, | al. 2009) | (I) Blood |
| | | | Nemanov et al. | (I) DLPFC | MetaSuicide |
| | | | 2005) | SZ (Hashimoto, | (Niculescu, Le- |
| | | | Psychosis | Straub et al. 2004) | Niculescu et al. |
| | | | (Bousman, Yung | (I) Hippocampus | 2017) |
| | | | et al. 2013) | SZ (Sheng, | (I) Blood |
| | | | SZ (Mostaid, Lee | Demers et al. 2012) | Pain (Niculescu, |
| | | | et al. 2017) | (I) PFC | Le-Niculescu et |
| | | | (Stefansson, | SZ (Hahn, Wang | al. 2019) |
| | | | Sarginson et al. | et al. 2006) | (I) Blood |
| | | | 2003, Zhao, Shi et | (I) PFC | Psychosis |
| | | | al. 2004) | SZ (Chong, | (Kurian, Le- |
| | | | (Williams, Preece | Thompson et al. | Niculescu et al. |
| | | | et al. 2003, Yang, | 2008) | 2011) |
| | | | Si et al. 2003, | (I) PFC (BA-9) | (I) glt8d1SH- |
| | | | Bakker, | SZ (Tkachev, | SY5Y cells |
| | | | Hoogendoorn et | Mimmack et al. 2003) | Stimulants(Fernandez- |
| | | | al. 2004, Mata, | (I) Brain | Castillo, |
| | | | Perez-Iglesias et | SZ (Law, Wang | Cabana- |
| | | | al. 2009, Rethelyi, | et al. 2012) | Dominguez et al. |
| | | | Bakker et al. | (I) BA 9, | 2015) |
| | | | 2010, So, Fong et | Hippocampus | (I) Blood |
| | | | al. 2010, Walker, | SZ (Marballi, Cruz | Stress (Le- |
| | | | Christoforou et al. | et al. 2012) | Niculescu, |
| | | | 2010 | | Roseberry et al. |
| | | | Greenwood, | | 2019) |
| | | | Lazzeroni et al. | | (I) Peripheral |
| | | | 2011, Shi, Yang | | blood monocytes |
| | | | et al. 2013, Yu, Bi | | Stress (Miller, |
| | | | et al. 2014) (van | | Chen et al. 2008) |
| | | | Schijndel, van | | (I) Leukocytes |
| | | | Loo et al. 2009, | | SZ (Petryshen, |
| | | | Papiol, Begemann | | Middleton et al. |
| | | | et al. 2011) | | 2005) |
| | | | (Pe | | (I) Blood |
| | | | tryshen, | | SZ (Vawter, |
| | | | Middleton et al. | | Philibert et al. |
| | | | 2005, Lachman, | | 2018 |
| | | | Pedrosa et al. | | (I) Fibroblast SZ |
| | | | 2006, Thomson, | | (Brennand, |
| | | | Christoforou et al. | | Simone et al. |
| | | | 2007, Georgieva, | | 2011) |
| | | | Dimitrova et al. | | (I) Lymphocyte |
| | | | 2008, Hong, | | SZ (Middleton, |
| | | | Wonodi et al. | | Pato et al. 2005) |
| | | | 2008, | | |
| | | | Hatzimanolis, | | |
| | | | McGrath et al. | | |
| | | | 2013) (Hanninen, | | |
| | | | Katila et al. 2008) | | |
| | | | Suicide | | |
| | | | (Sokolowski, | | |
| | | | Wasserman et al. | | |
| | | | 2016) | | |
| CD47 | 213856_at | (I) | Sleep (Jansen, | (D) Entorhinal cortex | (D) Blood |
| CD47 | | AP/4 | Watanabe et al. | Dementia (Patel, | Aging (Peters, |
| Molecule | | 66.7% | 2019) | Hodges et al. 2019) | Joehanes et al. |
| | | | | | 2015) |
| | | | | | (D) Blood |
| | | | | | Female Suicide |
| | | | | | (Levey, Niculescu |
| | | | | | et al. 2016) |
| | | | | | (D) Blood |
| | | | | | Male Suicide |
| | | | | | (Niculescu, Levey |
| | | | | | et al. 2015) |
| | | | | | (D) Blood |
| | | | | | MetaSuicide |
| | | | | | (Niculescu, Le- |
| | | | | | Niculescu et al. |
| | | | | | 2017) |
| | | | | | (D) Blood |
| | | | | | Pain (Jin, |
| | | | | | Zhang et al. 2013) |
| | | | | | (D) Peripheral |
| | | | | | blood monocytes |
| | | | | | Stress (Miller, |
| | | | | | Chen et al. 2008) |
| | | | | | (D) |
| | | | | | Neuroblastoma |
| | | | | | SZ (Cameron, |
| | | | | | Blake et al. 2019) |
| | | | | | (D) Blood |
| | | | | | SZ (Kuzman, |
| | | | | | Medved et al. |
| | | | | | 2009) |
| | | | | | (D) Blood |
| | | | | | Memory |
| | | | | | retention |
| | | | | | {Niculescu, 2019 |
| | | | | | #48328} |
| OLFM1 | 210924_at | (D) | | (I) Brain, | (I) Blood |
| Olfactomedin 1 | | DE/2 | | Orbitofrontal cortex | PTSD (Mehta, |
| | | 33.7% | | Suicide (Thalmeier, | Klengel et al. |
| | | | | Dickmann et al. 2008) | 2013) |
| | | | | (I) Brain | (I) Blood |
| | | | | Suicide (Thalmeier, | Aging (Peters, |
| | | | | Dickmann et al. 2008) | Joehanes et al. |
| | | | | (I) dIPFC | 2015) |
| | | | | (left hemisphere, | (I) Blood |
| | | | | Broadman area 46) | Memory |
| | | | | SZ (Hakak, Walker | retention |
| | | | | et al. 2001) | {Niculescu, 2019 |
| | | | | (I) Frontal Lobe | #48328} |
| | | | | Alcohol (Lewohl, | |
| | | | | Wang et al. 2000) | |
| SMAD7 | 204790_at | (I) | Aging (Levine | (D) Cerebellum | (I) |
| SMAD | | DE/2 | and Crimmins | Aging (Peters, | Blood Aging |
| Family | | 42.7% | 2016) | Joehanes et al. 2015) | (Peters, Joehanes |
| Member 7 | | (I) | SZ (Gandal, | (D) Parietal Cortex | et al. 2015) |
| | | AP/4 | Haney et al. 2018) | Dementia (Mills, | (D) Blood |
| | | 54% | | Nalpathamkalam et al. | Female Suicide |
| | | | | 2013) | (Levey, Niculescu |
| | | | | (D) dlPFC SZ | et al. 2016) |
| | | | | 31073119 | (D) Blood |
| | | | | | Stress (Le- |
| | | | | | Niculescu, |
| | | | | | Roseberry et al. |
| | | | | | 2019) |
| SLC6A4 | 242009_at | (D) | Alcohol (Feinn, | (I) Hippocampus | (Hypermethylated) |
| solute | | DE/4 | Nellissery et al. | Suicide (Gross- | Blood Stress |
| carrier | | 64.1% | 2005, Guo, | Isseroff, Israeli et al. | (Peng, Zhu et al. |
| family 6 | | | Wilhelmsen et al. | 1989) | 2018) |
| (neurotransmitter | | | 2007, | | (I) Blood |
| transporter), | | | Seneviratne, | | Stress, Anxiety |
| member 4 | | | Huang et al. | | (Azadmarzabadi, |
| | | | 2009) | | Haghighatfard et |
| | | | (Bordukalo- | | al. 2018) |
| | | | Niksic, Stefulj et | | (I) Blood |
| | | | al. 2012) | | Stress (Le- |
| | | | Aging (Gondo, | | Niculescu, |
| | | | Hirose et al. 2005, | | Roseberry et al. |
| | | | Li, Liu et al. | | 2019) |
| | | | 2014, Druley, | | (I) Blood |
| | | | Wang et al. 2016) | | Pain (Niculescu, |
| | | | Anxiety (Mizuno, | | Le-Niculescu et |
| | | | Aoki et al. 2006, | | al. 2019) |
| | | | Costas, Gratacos | | (I) |
| | | | et al. 2010) | | Lymphoblastoid |
| | | | (Wray, James et | | OCD (Hu, |
| | | | al. 2009, Forstner, | | Lipsky et al. |
| | | | Rambau et al. | | 2006) |
| | | | 2017) | | (I) Blood |
| | | | OCD | | MetaSuicide |
| | | | (Denys, Van | | (Niculescu, Le- |
| | | | Nieuwerburgh et | | Niculescu et al. |
| | | | al. 2006, | | 2017) |
| | | | Wendland, Moya | | (I) Blood |
| | | | et al. 2008) | | Female Suicide |
| | | | (Ozaki, Goldman | | (Levey, Niculescu |
| | | | et al. 2003, Lin | | et al. 2016) |
| | | | 2007, Wendland, | | (I) Blood |
| | | | Kruse et al. 2007) | | Alcohol |
| | | | (Voyiaziakis, | | (Seneviratne and |
| | | | Evgrafov et al. | | Johnson 2012) |
| | | | 2011) | | (I) PBMCs |
| | | | (Saiz, Garcia- | | Aging (Harris, |
| | | | Portilla et al. | | Riggio et al. |
| | | | 2008) | | 2017) |
| | | | ASD | | (Hypermethylation) |
| | | | (Kistner-Griffin, | | Blood Early |
| | | | Brune et al. 2011) | | Life Stress (Peng, |
| | | | (Coutinho, Sousa | | Zhu et al. 2018) |
| | | | et al. 2007, Ma, | | (D) CSF |
| | | | Rabionet et al. | | Suicide |
| | | | 2010, Gandal, | | (Samuelsson, |
| | | | Haney et al. 2018) | | Jokinen et al. |
| | | | (Zaboli, Jonsson | | 2006) |
| | | | et al. 2008, Hung, | | Aging (Harris, |
| | | | Lung et al. 2011) | | Riggio et al. |
| | | | (Bayle, Leroy et | | 2017) |
| | | | al. 2003) | | |
| | | | (De Luca, Zai et | | |
| | | | al. 2006, | | |
| | | | Lindholm | | |
| | | | Carlstrom, Saetre | | |
| | | | et al. 2012) | | |
| | | | Behavior(Vassos, | | |
| | | | Collier et al. | | |
| | | | 2014) | | |
| | | | Personality (Sen, | | |
| | | | Burmeister et al. | | |
| | | | 2004, Perroud, | | |
| | | | Salzmann et al. | | |
| | | | 2010) | | |
| | | | Pain | | |
| | | | (Offenbaecher, | | |
| | | | Bondy et al. | | |
| | | | 1999) | | |
| | | | (Cui, Yu et al. | | |
| | | | 2014) | | |
| | | | (Treister, Pud et | | |
| | | | al. 2011, James | | |
| | | | 2013, Zorina- | | |
| | | | Lichtenwalter, | | |
| | | | Meloto et al. | | |
| | | | 2016, Tour, | | |
| | | | Lofgren et al. | | |
| | | | 2017) | | |
| | | | Panic (Maron, | | |
| | | | Lang et al. 2005, | | |
| | | | Lonsdorf, Ruck et | | |
| | | | al. 2009, Gyawali, | | |
| | | | Subaran et al. | | |
| | | | 2010, Strug, | | |
| | | | Suresh et al. | | |
| | | | 2010) | | |
| | | | Stress | | |
| | | | (Kilpatrick, | | |
| | | | Koenen et al. | | |
| | | | 2007) | | |
| | | | (Lee, Lee et al. | | |
| | | | 2005) | | |
| | | | PTSD (Zhang, | | |
| | | | Qu et al. 2017) | | |
| | | | Suicide (Caspi, | | |
| | | | Sugden et al. | | |
| | | | 2003) (Bondy, | | |
| | | | Erfurth et al. | | |
| | | | 2000, Joiner, | | |
| | | | Johnson et al. | | |
| | | | 2002, Anguelova, | | |
| | | | Benkelfat et al. | | |
| | | | 2003, Campi- | | |
| | | | Azevedo, Boson | | |
| | | | et al. 2003, | | |
| | | | Hranilovic, | | |
| | | | Stefulj et al. 2003, | | |
| | | | Courtet, Picot et | | |
| | | | al. 2004, Jernej, | | |
| | | | Stefulj et al. 2004, | | |
| | | | Gaysina, | | |
| | | | Zainullina et al. | | |
| | | | 2006, Gibb, | | |
| | | | McGeary et al. | | |
| | | | 2006, Li and He | | |
| | | | 2007, Roy, Hu et | | |
| | | | al. 2007, | | |
| | | | Wasserman, | | |
| | | | Geijer et al. 2007, | | |
| | | | Bah, Lindstrom et | | |
| | | | al. 2008, Akar, | | |
| | | | Sayin et al. 2010, | | |
| | | | Cicchetti, | | |
| | | | Rogosch et al. | | |
| | | | 2010, Perroud, | | |
| | | | Salzmann et al. | | |
| | | | 2010, Saiz, | | |
| | | | Garcia-Portilla et | | |
| | | | al. 2011, Clayden, | | |
| | | | Zaruk et al. 2012, | | |
| | | | Dell'osso, | | |
| | | | Mandelli et al. | | |
| | | | 2013, Schneider, | | |
| | | | El Hajj et al. | | |
| | | | 2015) | | |

| | | Prior | | | |
|---|---|---|---|---|---|
| | Prior | Non- | Prior | CFG | |
| Gene | Non- | human | Non- | Score | |
| Symbol/ | human | Brain | human | For | Step |
| Gene | Genetic | Tissue | Peripheral | Other | 1-4 |
| Name | Evidence | Evidence | Evidence | Disorders | CFE |
| DOCK10 | | (D) Nac | | 10 | 24 |
| dedicator of | | Social Defeat | | | |
| cytokinesis 10 | | (Bagot, Cates | | | |
| | | et al. 2017) | | | |
| | | (D) Female | | | |
| | | Nac Stress | | | |
| | | (Labonte, | | | |
| | | Engmann et | | | |
| | | al. 2017) | | | |
| GLS | | (D) | | 8 | 24 |
| Glutaminase | | CP (paradigm | | | |
| | | 2) Alcohol | | | |
| | | (Rodd, | | | |
| | | Bertsch et al. | | | |
| | | 2007 | | | |
| | | Hippocampus | | | |
| | | Anxiety | | | |
| | | (Mehta, Wang | | | |
| | | et al. 2013) | | | |
| | | (D) MPFC | | | |
| | | PTSD | | | |
| | | (Muhie, | | | |
| | | Gautam et al. | | | |
| | | 2015) | | | |
| PRPS1 | | | | 9 | 24 |
| Phosphoribosyl | | | | | |
| Pyrophosphate | | | | | |
| Synthetase 1 | | | | | |
| TMEM161B | | (D) PFC | | 10 | 24 |
| Transmembrane | | Stress | | | |
| Protein | | (van Heerden, | | | |
| 161B | | Conesa et al. | | | |
| | | 2009) | | | |
| | | (I) | | | |
| | | NAC, PFC | | | |
| | | Female | | | |
| | | Stress | | | |
| | | (Labonte, | | | |
| | | Engmann et | | | |
| | | al. 2017) | | | |
| GLO1 | | (D) | | 11.5 | 22.5 |
| Glyoxalase I | | Hypothalamic | | | |
| | | PVN Anxiety | | | |
| | | (Kromer, | | | |
| | | Kessler et al. | | | |
| | | 2005) | | | |
| | | (D) | | | |
| | | AMY, | | | |
| | | CINGULATE | | | |
| | | CORTEX, | | | |
| | | HIPPOCAMPUS, | | | |
| | | ETC. | | | |
| | | Anxiety | | | |
| | | (Hovatta, | | | |
| | | Tennant et al. | | | |
| | | 2005) | | | |
| | | (D) Cortex | | | |
| | | Dementia | | | |
| | | (Castillo, | | | |
| | | Leon et al. | | | |
| | | 2017) | | | |
| | | (D) | | | |
| | | Hippocampus | | | |
| | | Depression | | | |
| | | Susceptible | | | |
| | | (Tang, Huang | | | |
| | | et al. 2019) | | | |
| FANCF | | (D) | | 8 | 22 |
| Fanconi | | AMY (males) | | | |
| Anemia | | Stress (Le- | | | |
| Complementation | | Niculescu, | | | |
| Group F | | McFarland et | | | |
| | | al. 2008) | | | |
| HNRNPDL | | (D) | (D) | 10 | 22 |
| Heterogeneous | | AMY | Lymphocytes | | |
| Nuclear | | Anxiety | Hallucinogens | | |
| Ribonucleoprotein | | (Le- | (Le- | | |
| D Like | | Niculescu, | Niculescu, | | |
| | | Balaraman et | Balaraman et | | |
| | | al. 2011) | al. 2007) | | |
| | | (D) | (D) Spinal | | |
| | | VT | cord and | | |
| | | Hallucinogens | DRG-RNA | | |
| | | (Le- | Pain | | |
| | | Niculescu, | (Sandercock, | | |
| | | Balaraman et | Barnett et al. | | |
| | | al. 2007) | 2019) | | |
| | | (D) PFC | (D) Blood | | |
| | | Stress (Malki, | Stimulants | | |
| | | Tosto et al. | (Le- | | |
| | | 2016) | Niculescu, | | |
| | | | Kurian et al. | | |
| | | | 2009) | | |
| | | | (I) | | |
| | | | Lymphocytes | | |
| | | | Hallucinogens | | |
| | | | (Le- | | |
| | | | Niculescu, | | |
| | | | Balaraman et | | |
| | | | al. 2007) | | |
| NRG1 | SZ | (I) mPFC, | (I) Plasma | 10 | 22 |
| Neuregulin | (Chen, | PL Cortex | Aging | | 26 |
| 1 | Johnson | Behavior | (Lehallier, | | |
| | et al. | (Chen, Lan et | Gate et al. | | |
| | 2008) | al. 2017) | 2019) | | |
| | (Stefansson, | (I) Prefrontal | | | |
| | Sigurdsson | cortical tissue | | | |
| | et al. | Memory, SZ | | | |
| | 2002. | (Olaya, | | | |
| | O'Tuathaigh, | Heusner et al. | | | |
| | Babovic | 2018) | | | |
| | et al | (I) | | | |
| | 2007. | AMY | | | |
| | Long, | Stress | | | |
| | Chesworth | (Zhang, Li et | | | |
| | et al. | al. 2015) | | | |
| | 2012. | (I) Prefrontal | | | |
| | Papaleo, | cortex SZ | | | |
| | Yang et | (Elfving, | | | |
| | al. 2016) | Muller et al. | | | |
| | | 2019) | | | |
| | | (I) | | | |
| | | Medial | | | |
| | | Prefrontal | | | |
| | | Cortex SZ | | | |
| | | (Papaleo, | | | |
| | | Yang et al. | | | |
| | | 2016) | | | |
| CD47 | | | | 8.00 | 21 |
| CD47 | | | | | |
| Molecule | | | | | |
| OLFM1 | Alcohol | (I) | | 10 | 21 |
| Olfactomedin 1 | (Lo, | NAC | | | |
| | Lossie et | Hallucinogens | | | |
| | al. 2016) | (Le- | | | |
| | | Niculescu, | | | |
| | | Balaraman et | | | |
| | | al. 2007) | | | |
| | | (I) Cerebral | | | |
| | | Cortex SZ | | | |
| | | (Ouchi, | | | |
| | | Kubota et al. | | | |
| | | 2005) | | | |
| | | (I) PFC | | | |
| | | SZ | | | |
| | | (Miller, Zeier | | | |
| | | et al. 2012) | | | |
| | | (I) Female | | | |
| | | NAC Stress | | | |
| | | (Labonte, | | | |
| | | Engmann et | | | |
| | | al. 2017) | | | |
| SMAD7 | | (D) | | 9 | 21 |
| SMAD | | AMY Stress | | | |
| Family | | (Kim and Han | | | |
| Member 7 | | 2006) | | | |
| | | (D) Male | | | |
| | | Nac Stress | | | |
| | | (Labonte, | | | |
| | | Engmann et | | | |
| | | al. 2017) | | | |
| SLC6A4 | Alcohol | (I) | (I) | 10 | 20 |
| solute | (Lo, | Hippocampus | Lymphocytes | | |
| carrier | Lossie et | Alcohol | Hallucinogens | | |
| family 6 | al. 2016) | (de Almeida | (Le- | | |
| (neurotransmitter | ASD | Magalhaes, | Niculescu, | | |
| transporter), | (Jones, | Correia et al. | Balaraman et | | |
| member 4 | Smith et | 2018) | al. 2007) | | |
| | al. 2010) | (I) | | | |
| | | Hippocampus | | | |
| | | Anxiety | | | |
| | | (Zhang, | | | |
| | | Amstein et al. | | | |
| | | 2005) | | | |

We also tested an algorithm (UP-Mood) combining as predictors BioM26, along with mood (SMS7) and with a measure of clinical severity of bipolar disorder (CFI-BP, Figure), with modest synergistic effects (Table 14). Of note, CFI-BP was a good predictor of all future hospitalizations for mania in all (Cox regression OR of 2.9 (p=2.5E-04)), and an even better predictor in males with bipolar disorder (OR of 3.2 (p=8.3E-05)).

TABLE 8
Pharmacogenomics: biomarkers for low mood/depression (BioM12 depression).

| | | Discovery | | |
|---|---|---|---|---|
| | | in Blood | | |
| Gene | | (Direction of Change | | |
| Symbol/ | | in High Mood) | | |
| Gene | | Method/Score/% | | |
| Name | Probesets | 6 pt | Omega-3 | Antidepressants |
| NRG1 | 208230_s_at | (D) | | (D) |
| neuregulin 1 | | DE/2 | | PBMC |
| | | 33.7% | | Antidepressants |
| | | | | (Belzeaux, |
| | | | | Formisano- |
| | | | | Treziny et al. |
| | | | | 2010) |
| | | | | Antidepressants |
| | | | | (Biernacka, |
| | | | | Sangkuhl et al. |
| | | | | 2015) |
| DOCK10 | 219279_at | (I) | | (I)NAC |
| dedicator of | | DE/2 | | Ketamine(Bagot, |
| cytokinesis 10 | | 41.5% | | Cates et al. |
| | | | | 2017) |
| GLS | 203159_at | (I) | (I) HIP | |
| glutaminase | | DE/4 | (males) | |
| | | 53.7% | Omega-3 | |
| | | | fatty | |
| | | | acids | |
| | | | (Le- | |
| | | | Niculescu, | |
| | | | Case et al. | |
| | | | 2011) | |
| PRPS1 | 209440_at | (I) | | |
| Phosphoribosyl | | DE/4 | | |
| Pyrophosphate | | 57.3% | | |
| Synthetase 1 | | | | |
| TMEM161B | 227861_at | (I) | | |
| Transmembrane | | AP/4 | | |
| Protein 161B | | 62.1% | | |
| GLO1 | 200681_at | (I) | (I) | |
| glyoxalase I | | DE/2 | Lymphocytes | |
| | | 41.5% | Omega-3 | |
| | | | fatty | |
| | | | acids | |
| | | | (Le- | |
| | | | Niculescu, | |
| | | | Case et al. | |
| | | | 2011) | |
| FANCF | 218689_at | (I) | | |
| Fanconi | | DE/4 | | |
| Anemia | | 54.9% | | |
| Complementation | | | | |
| Group F | | | | |
| HNRNPDL | 212454_x_at | (I) | (I) NAC | |
| heterogeneous | | DE/2 | (females) | |
| nuclear | | 35.4% | Omega-3 | |
| ribonucleoprotein | | | fatty | |
| D like | | | acids | |
| | | | (Le- | |
| | | | Niculescu, | |
| | | | Case et al. | |
| | | | 2011) | |
| NRG1 | 208232_x_at | (D) | | (D) |
| neuregulin 1 | | AP/4 | | PBMC |
| Most | | 60.7% | | Antidepressants |
| Reproducible | | | | (Belzeaux, |
| predictors | | | | Formisano- |
| | | | | Treziny et al. |
| | | | | 2010) |
| | | | | Antidepressants |
| | | | | (Biernacka, |
| | | | | Sangkuhl et al. |
| | | | | 2015) |
| CD47 | 213856_at | (I) | (I) | (I) Frontal |
| CD47 | | AP/4 | Lymphocytes | Cortex |
| molecule | | 66.7% | (males) | Venlafaxine |
| | | | Omega-3 | (Tamasi, |
| | | | fatty | Petschner et al. |
| | | | acids | 2014) |
| | | | (Le- | |
| | | | Niculescu, | |
| | | | Case et al. | |
| | | | 2011) | |
| OLFM1 | 210924_at | (D) | | |
| Olfactomedin | | DE/2 | | |
| 1 | | 33.7% | | |
| SMAD7 | 204790_at | (I) | | |
| SMAD family | | DE/2 | | |
| member 7 | | 42.7% | | |
| | | (I) | | |
| | | AP/4 | | |
| | | 54.0% | | |
| SLC6A4 | 242009_at | (D) | (D) | (D) |
| solute carrier | | DE/4 | Lymphocytes | Neural |
| family 6 | | 64.1% | (females) | progenitor cells |
| (neurotransmitter | | | Omega-3 | (NPCs) |
| transporter), | | | fatty | Imipramine, |
| member 4 | | | acids | Citalopram |
| | | | (Le- | (Lopez, |
| | | | Niculescu, | Lim |
| | | | Case et al. | et al. |
| | | | 2011) | 2014) |

| Gene | | | | Step |
|---|---|---|---|---|
| Symbol/ | Lithium and | | | 1-4 |
| Gene | Other Mood | Anti- | | CFE |
| Name | Stabilizers | psychotics | Others | Score |
| NRG1 | (NA) | (D)blood | | 26 |
| neuregulin 1 | SH-SY5Y | Antipsychotic | | |
| | neuroblastoma | with | | |
| | cells | weight | | |
| | Lithium | gain in | | |
| | (Gow, | men | | |
| | Mirembe et al. | (Sainz, | | |
| | 2013) | Prieto et al. | | |
| | (D) | 2019) | | |
| | Neuroblastoma | | | |
| | cells | | | |
| | Valproate | | | |
| | (Warburton, | | | |
| | Savage et al. | | | |
| | 2015) | | | |
| DOCK10 | | | (I) | 24 |
| dedicator of | | | HIP | |
| cytokinesis 10 | | | Physical | |
| | | | and | |
| | | | Cognitive | |
| | | | stimulation | |
| | | | (Huttenrauch. | |
| | | | Salinas | |
| | | | et al. | |
| | | | 2016) | |
| GLS | | (I) VT | CB-839 | 24 |
| glutaminase | | Clozapine | | |
| | | (Le- | | |
| | | Niculescu, | | |
| | | Balaraman | | |
| | | et al. 2007) | | |
| | | (I) | | |
| | | Striatum | | |
| | | Risperidone | | |
| | | (Lanz, | | |
| | | Reinhart et | | |
| | | al. 2019) | | |
| PRPS1 | (I)Lymphoblastiod | | | 24 |
| Phosphoribosyl | cells | | | |
| Pyrophosphate | Lithium | | | |
| Synthetase 1 | (Chen, Wang | | | |
| | et al. 2009) | | | |
| TMEM161B | | | | 24 |
| Transmembrane | | | | |
| Protein 161B | | | | |
| GLO1 | | | | 22.5 |
| glyoxalase I | | | | |
| FANCF | | | | 22 |
| Fanconi | | | | |
| Anemia | | | | |
| Complementation | | | | |
| Group F | | | | |
| HNRNPDL | | | (I) | 22 |
| heterogeneous | | | Lymphocytes | |
| nuclear | | | Diazepam | |
| ribonucleoprotein | | | (Le- | |
| D like | | | Niculescu, | |
| | | | Balaraman et | |
| | | | al. 2011) | |
| NRG1 | (NA) | (D)blood | | 22 |
| neuregulin 1 | SH-SY5Y | Antipsychotic | | |
| Most | neuroblastoma | with | | |
| Reproducible | cells | weight | | |
| predictors | Lithium | gain in | | |
| | (Gow, | men | | |
| | Mirembe et al. | (Sainz, | | |
| | 2013) | Prieto et al. | | |
| | (D) | 2019) | | |
| | Neuroblastoma | | | |
| | cells | | | |
| | Valproate | | | |
| | (Warburton, | | | |
| | Savage et al. | | | |
| | 2015) | | | |
| CD47 | (I)Corpus | (I) | hu5F9-G4, | 21 |
| CD47 | Collosum | Lymphocytes, | CC-90002, | |
| molecule | Lithium(Akkouh, | VT | ALX148, | |
| | Skrede et | Clozapine | TTI-621, | |
| | al. 2019) | (Le- | TTI-622 | |
| | | Niculescu, | | |
| | | Balaraman | | |
| | | et al. 2007) | | |
| OLFM1 | (D) NT2.D1 | | | 21 |
| Olfactomedin | cells | | | |
| 1 | Valproate | | | |
| | (Hill, Nagel et | | | |
| | al. 2013) | | | |
| SMAD7 | | | | 21 |
| SMAD family | | | | |
| member 7 | | | | |
| SLC6A4 | | | | 20 |
| solute carrier | | | | |
| family 6 | | | | |
| (neurotransmitter | | | | |
| transporter), | | | | |
| member 4 | | | | |

TABLE 9
Drug repurposing- Connectivity Map for Top Candidate Biomarkers
CFE3 Score of 16 and above. Direction of high mood.

| rank | cmap name | score |
|---|---|---|
| 1 | isoflupredone | 1 |
| 2 | adiphenine | 0.967 |
| 3 | tropine | 0.892 |
| 4 | fludrocortisone | 0.884 |
| 5 | flucloxacillin | 0.88 |
| 6 | piperine | 0.863 |
| 7 | trimethobenzamide | 0.832 |
| 8 | isoxicam | 0.826 |
| 9 | tiletamine | 0.819 |
| 10 | metaraminol | 0.817 |
| 11 | acemetacin | 0.816 |
| 12 | sulfabenzamide | 0.816 |
| 13 | chlorogenic acid | 0.816 |
| 14 | 15-delta | 0.813 |
| | prostaglandin J2 | |
| 15 | saquinavir | 0.81 |
| 16 | methanthelinium | 0.808 |
| | bromide | |
| 17 | cyclopenthiazide | 0.8 |
| 18 | molsidomine | 0.8 |
| 19 | metformin | 0.795 |
| 20 | genistein | 0.795 |
| 21 | bethanechol | 0.793 |
| 22 | bacampicillin | 0.791 |
| 23 | enoxacin | 0.791 |
| 24 | carbimazole | 0.789 |
| 25 | bemegride | 0.788 |

TABLE 10
Drug repurposing- NIH LINCS for Top Candidate Biomarkers
CFE3 Score of 16 and above. Direction of high mood.

| score | Drug |
|---|---|
| 0.2 | trichostatin A |
| 0.2 | vorinostat |
| 0.2 | KUC104135 KUC104135N |
| 0.2 | KM 00927 |
| 0.2 | BRD-K91370081 |
| 0.2 | BRD-K12867552 |
| 0.2 | BRD-A94377914 |
| 0.2 | AZD-8055 |
| 0.15 | PD 123319 |
| | ditrifluoroacetate |
| 0.15 | THM-I-94 |
| 0.15 | NNC 55-0396 |
| | dihydrochloride |
| 0.15 | IOPANIC ACID |
| 0.15 | NADOLOL |
| 0.15 | ROSUVASTATIN CALCIUM |
| 0.15 | BNTX maleate |
| 0.15 | KU 0060648 |
| | trihydrochloride |
| 0.15 | MLN4924 |
| 0.15 | 16-HYDROXYTRIPTOLIDE |
| 0.15 | TG101348 |
| 0.15 | BRD-K92317137 |
| 0.15 | QUINACRINE |
| | HYDROCHLORIDE |
| 0.15 | Dorsomorphin |
| | dihydrochloride |
| 0.15 | HDAC6 inhibitor ISOX |
| 0.15 | PP-110 |
| 0.15 | KCR-13 |
| 0.15 | geldanamycin |
| 0.15 | NCGC00241077-01 |
| 0.15 | BRD-K10846167 |
| 0.15 | wortmannin |

TABLE 11
Top candidate biomarkers after steps 1-3 (Discovery, Prioritization, Validation).
Expanded list with CFE3 score of 14 and above.

| | GeneCards | Gene | AP | DE | Discovery | Prioritizations | Validation | |
|---|---|---|---|---|---|---|---|---|
| Probeset | Symbol | Name | Change | change | Score | Score | Score | CFE3 |
| 227861_at | TMEM161B | Transmembrane | I | | 4 | 10.00 | 4 | 18.00 |
| | | Protein 161B | | | | | | |
| 200681_at | GLO1 | Glyoxalase I | | I | 2 | 11.50 | 4 | 17.50 |
| 209440_at | PRPS1 | Phosphoribosyl | | I | 4 | 9.00 | 4 | 17.00 |
| | | Pyrophosphate | | | | | | |
| | | Synthetase 1 | | | | | | |
| 208447_s_at | PRPS1 | Phosphoribosyl | | I | 4 | 9.00 | 4 | 17.00 |
| | | Pyrophosphate | | | | | | |
| | | Synthetase 1 | | | | | | |
| 204790_at | SMAD7 | SMAD Family | I | I | 4 | 9.00 | 4 | 17.00 |
| | | Member 7 | | | | | | |
| 206385_s_at | ANK3 | Ankyrin 3 | | I | 4 | 11.00 | 2 | 17.00 |
| 207563_s_at | OGT | O-Linked N- | | I | 4 | 11.00 | 2 | 17.00 |
| | | Acetylglucosamine | | | | | | |
| | | (GlcNAc) | | | | | | |
| | | Transferase | | | | | | |
| 213856_at | CD47 | CD47 Molecule | I | | 4 | 8.00 | 4 | 16.00 |
| 203159_at | GLS | Glutaminase | | I | 4 | 8.00 | 4 | 16.00 |
| 226529_at | TMEM106B | Transmembrane | | I | 2 | 10.00 | 4 | 16.00 |
| | | Protein 106B | | | | | | |
| 212039_x_at | RPL3 | Ribosomal Protein | | I | 4 | 8.00 | 4 | 16.00 |
| | | L3 | | | | | | |
| 218689_at | FANCF | Fanconi Anemia | | I | 4 | 8.00 | 4 | 16.00 |
| | | Complementation | | | | | | |
| | | Group F | | | | | | |
| 212454_x_at | HNRNPDL | Heterogeneous | | I | 2 | 10.00 | 4 | 16.00 |
| | | Nuclear | | | | | | |
| | | Ribonucleoprotein | | | | | | |
| | | D Like | | | | | | |
| 219279_at | DOCK10 | Dedicator Of | | I | 2 | 10.00 | 4 | 16.00 |
| | | Cytokinesis 10 | | | | | | |
| 209563_x_at | CALM1 | Calmodulin 1 | | I | 4 | 10.00 | 2 | 16.00 |
| 208230_s_at | NRG1 | Neuregulin 1 | | D | 2 | 10.00 | 4 | 16.00 |
| 210924_at | OLFM1 | Olfactomedin 1 | | D | 2 | 10.00 | 4 | 16.00 |
| 242009_at | SLC6A4 | Solute Carrier | | D | 4 | 10.00 | 2 | 16.00 |
| | | Family 6 Member | | | | | | |
| | | 4 | | | | | | |
| 223498_at | SPECC1 | Sperm Antigen | | D | 4 | 10.00 | 2 | 16.00 |
| | | With Calponin | | | | | | |
| | | Homology And | | | | | | |
| | | Coiled-Coil | | | | | | |
| | | Domains 1 | | | | | | |
| 224818_at | SORT1 | Sortilin 1 | D | D | 4 | 10.00 | 2 | 16.00 |
| 1553859_at | TPH1 | Tryptophan | D | | 4 | 10.00 | 2 | 16.00 |
| | | Hydroxylase 1 | | | | | | |
| 242336_at | GSK3B | Glycogen | D | | 4 | 10.00 | 2 | 16.00 |
| | | Synthase Kinase 3 | | | | | | |
| | | Beta | | | | | | |
| 208232_x_at | NRG1 | Neuregulin 1 | D | | 4 | 10.00 | 2 | 16.00 |
| 201668_x_at | MARCKS | Myristoylated | | D | 4 | 10.00 | 2 | 16.00 |
| | | Alanine Rich | | | | | | |
| | | Protein Kinase C | | | | | | |
| | | Substrate | | | | | | |
| 201670_s_at | MARCKS | Myristoylated | | D | 4 | 10.00 | 2 | 16.00 |
| | | Alanine Rich | | | | | | |
| | | Protein Kinase C | | | | | | |
| | | Substrate | | | | | | |
| 241492_at | NR3C1 | Nuclear Receptor | | D | 6 | 10.00 | 0 | 16.00 |
| | | Subfamily 3 | | | | | | |
| | | Group C Member | | | | | | |
| | | 1 | | | | | | |
| 244272_s_at | TC2N | Tandem C2 | | I | 6 | 7.00 | 2 | 15.00 |
| | | Domains, Nuclear | | | | | | |
| 226922_at | RANBP2 | RAN Binding | I | I | 4 | 9.00 | 2 | 15.00 |
| | | Protein 2 | | | | | | |
| 222630_at | RFX7 | Regulatory Factor | | I | 4 | 9.00 | 2 | 15.00 |
| | | X7 | | | | | | |
| 226154_at | DNM1L | Dynamin 1 Like | | I | 4 | 9.00 | 2 | 15.00 |
| 209240_at | OGT | O-Linked N- | | I | 2 | 11.00 | 2 | 15.00 |
| | | Acetylglucosamine | | | | | | |
| | | (GlcNAc) | | | | | | |
| | | Transferase | | | | | | |
| 203685_at | BCL2 | BCL2, Apoptosis | | I | 2 | 11.00 | 2 | 15.00 |
| | | Regulator | | | | | | |
| 207564_x_at | OGT | O-Linked N- | | I | 2 | 11.00 | 2 | 15.00 |
| | | Acetylglucosamine | | | | | | |
| | | (GlcNAc) | | | | | | |
| | | Transferase | | | | | | |
| 206641_at | TNFRSF17 | TNF Receptor | I | | 6 | 7.00 | 2 | 15.00 |
| | | Superfamily | | | | | | |
| | | Member 17 | | | | | | |
| 235603_at | HNRNPU | Heterogeneous | I | | 4 | 9.00 | 2 | 15.00 |
| | | Nuclear | | | | | | |
| | | Ribonucleoprotein | | | | | | |
| | | U | | | | | | |
| 230267_at | WSB1 | WD Repeat And | I | | 4 | 9.00 | 2 | 15.00 |
| | | SOCS Box | | | | | | |
| | | Containing 1 | | | | | | |
| 215706_x_at | ZYX | Zyxin | | D | 4 | 9.00 | 2 | 15.00 |
| 209791_at | PADI2 | Peptidyl Arginine | D | D | 4 | 9.00 | 2 | 15.00 |
| | | Deiminase 2 | | | | | | |
| 228748_at | CD59 | CD59 Molecule | | D | 4 | 9.00 | 2 | 15.00 |
| | | (CD59 Blood | | | | | | |
| | | Group) | | | | | | |
| 203382_s_at | APOE | Apolipoprotein E | | D | 4 | 9.00 | 2 | 15.00 |
| 202700_s_at | TMEM63A | Transmembrane | | D | 4 | 9.00 | 2 | 15.00 |
| | | Protein 63A | | | | | | |
| 1561365_at | NRP1 | Neuropilin 1 | | D | 4 | 9.00 | 2 | 15.00 |
| 225925_s_at | USP48 | Ubiquitin Specific | | I | 6 | 5.00 | 4 | 15.00 |
| | | Peptidase 48 | | | | | | |
| 232262_at | PIGL | Phosphatidylinositol | I | I | 6 | 5.00 | 4 | 15.00 |
| | | Glycan Anchor | | | | | | |
| | | Biosynthesis Class L | | | | | | |
| 207606_s_at | ARHGAP12 | Rho GTPase | I | I | 4 | 7.00 | 4 | 15.00 |
| | | Activating Protein | | | | | | |
| | | 12 | | | | | | |
| 214060_at | SSBP1 | Single Stranded | | I | 4 | 7.00 | 4 | 15.00 |
| | | DNA Binding | | | | | | |
| | | Protein 1 | | | | | | |
| 232914_s_at | SYTL2 | Synaptotagmin | | I | 4 | 7.00 | 4 | 15.00 |
| | | Like 2 | | | | | | |
| 205006_s_at | NMT2 | N- | | I | 4 | 7.00 | 4 | 15.00 |
| | | Myristoyltransferase | | | | | | |
| | | 2 | | | | | | |
| 204937_s_at | ZNF274 | Zinc Finger | | I | 4 | 7.00 | 4 | 15.00 |
| | | Protein 274 | | | | | | |
| 218699_at | RAB29 | RAB29, Member | | I | 4 | 7.00 | 4 | 15.00 |
| | | RAS Oncogene | | | | | | |
| | | Family | | | | | | |
| 227410_at | FAM43A | Family With | | I | 4 | 7.00 | 4 | 15.00 |
| | | Sequence | | | | | | |
| | | Similarity 43 | | | | | | |
| | | Member A | | | | | | |
| 205901_at | PNOC | Prepronociceptin | | I | 4 | 7.00 | 4 | 15.00 |
| 204651_at | NRF1 | Nuclear | | I | 4 | 7.00 | 4 | 15.00 |
| | | Respiratory | | | | | | |
| | | Factor 1 | | | | | | |
| 1552733_at | KLHDC1 | Kelch Domain | I | I | 4 | 7.00 | 4 | 15.00 |
| | | Containing 1 | | | | | | |
| 229256_at | PGM2L1 | Phosphoglucomutase | | I | 4 | 7.00 | 4 | 15.00 |
| | | 2 Like 1 | | | | | | |
| 202978_s_at | CREBZF | CREB/ATF BZIP | | I | 4 | 7.00 | 4 | 15.00 |
| | | Transcription | | | | | | |
| | | Factor | | | | | | |
| 213853_at | DNAJC24 | DnaJ Heat Shock | | I | 4 | 7.00 | 4 | 15.00 |
| | | Protein Family | | | | | | |
| | | (Hsp40) Member | | | | | | |
| | | C24 | | | | | | |
| 212486_s_at | FYN | FYN Proto- | | I | 2 | 9.00 | 4 | 15.00 |
| | | Oncogene, Src | | | | | | |
| | | Family Tyrosine | | | | | | |
| | | Kinase | | | | | | |
| 213134_x_at | BTG3 | BTG Anti- | I | | 2 | 9.00 | 4 | 15.00 |
| | | Proliferation | | | | | | |
| | | Factor 3 | | | | | | |
| 1558702_at | TEX10 | Testis Expressed | I | | 4 | 7.00 | 4 | 15.00 |
| | | 10 | | | | | | |
| 212217_at | PREPL | Prolyl | I | | 2 | 9.00 | 4 | 15.00 |
| | | Endopeptidase- | | | | | | |
| | | Like | | | | | | |
| 1559257_a_at | MAGI1 | Membrane | | D | 2 | 9.00 | 4 | 15.00 |
| | | Associated | | | | | | |
| | | Guanylate Kinase, | | | | | | |
| | | WW And PDZ | | | | | | |
| | | Domain | | | | | | |
| | | Containing 1 | | | | | | |
| 229982_at | QSER1 | Glutamine And | | D | 2 | 9.00 | 4 | 15.00 |
| | | Serine Rich 1 | | | | | | |
| 205166_at | CAPN5 | Calpain 5 | D | D | 4 | 7.00 | 4 | 15.00 |
| 233620_at | ARHGEF12 | Rho Guanine | | D | 4 | 7.00 | 4 | 15.00 |
| | | Nucleotide | | | | | | |
| | | Exchange Factor | | | | | | |
| | | 12 | | | | | | |
| 203472_s_at | SLCO2B1 | Solute Carrier | | D | 4 | 7.00 | 4 | 15.00 |
| | | Organic Anion | | | | | | |
| | | Transporter | | | | | | |
| | | Family Member | | | | | | |
| | | 2B1 | | | | | | |
| 230537_at | PCDH17 | Protocadherin 17 | | D | 4 | 7.00 | 4 | 15.00 |
| 1558552_s_at | PQLC2L | PQ Loop Repeat | | D | 4 | 7.00 | 4 | 15.00 |
| | | Containing 2 Like | | | | | | |
| 1555468_at | NRP2 | Neuropilin 2 | | D | 4 | 7.00 | 4 | 15.00 |
| 223036_at | FARSB | Phenylalanyl- | D | D | 4 | 7.00 | 4 | 15.00 |
| | | TRNA Synthetase | | | | | | |
| | | Beta Subunit | | | | | | |
| 203684_s_at | BCL2 | BCL2, Apoptosis | | I | 4 | 11.00 | 0 | 15.00 |
| | | Regulator | | | | | | |
| 1570190_at | LSAMP | Limbic System- | | D | 6 | 9.00 | 0 | 15.00 |
| | | Associated | | | | | | |
| | | Membrane | | | | | | |
| | | Protein | | | | | | |
| 224224_s_at | PDE11A | Phosphodiesterase | D | D | 4 | 8.50 | 2 | 14.50 |
| | | 11A | | | | | | |
| 228177_at | CREBBP | CREB Binding | | D | 4 | 10.50 | 0 | 14.50 |
| | | Protein | | | | | | |
| 209824_s_at | ARNTL | Aryl Hydrocarbon | | I | 2 | 10.00 | 2 | 14.00 |
| | | Receptor Nuclear | | | | | | |
| | | Translocator Like | | | | | | |
| 211535_s_at | FGFR1 | Fibroblast Growth | | D | 4 | 10.00 | 0 | 14.00 |
| | | Factor Receptor 1 | | | | | | |
| 225577_at | HCG18 | HLA Complex | | I | 4 | 8.00 | 2 | 14.00 |
| | | Group 18 (Non- | | | | | | |
| | | Protein Coding) | | | | | | |
| 222026_at | RBM3 | RNA Binding | | I | 4 | 8.00 | 2 | 14.00 |
| | | Motif (RNP1, | | | | | | |
| | | RRM) Protein 3 | | | | | | |
| 205376_at | INPP4B | Inositol | | I | 4 | 8.00 | 2 | 14.00 |
| | | Polyphosphate-4- | | | | | | |
| | | Phosphatase Type | | | | | | |
| | | II B | | | | | | |
| 212959_s_at | GNPTAB | N- | | I | 4 | 8.00 | 2 | 14.00 |
| | | Acetylglucosamine- | | | | | | |
| | | 1-Phosphate | | | | | | |
| | | Transferase Alpha | | | | | | |
| | | And Beta Subunits | | | | | | |
| 235537_at | OCIAD1 | OCIA Domain | | I | 4 | 8.00 | 2 | 14.00 |
| | | Containing 1 | | | | | | |
| 204622_x_at | NR4A2 | Nuclear Receptor | | I | 2 | 10.00 | 2 | 14.00 |
| | | Subfamily 4 | | | | | | |
| | | Group A Member | | | | | | |
| | | 2 | | | | | | |
| 226682_at | RORA | RAR Related | | I | 2 | 10.00 | 2 | 14.00 |
| | | Orphan Receptor | | | | | | |
| | | A | | | | | | |
| 201739_at | SGK1 | Serum/Glucocorticoid | | I | 2 | 10.00 | 2 | 14.00 |
| | | Regulated | | | | | | |
| | | Kinase 1 | | | | | | |
| 209067_s_at | HNRNPDL | Heterogeneous | | I | 2 | 10.00 | 2 | 14.00 |
| | | Nuclear | | | | | | |
| | | Ribonucleoprotein | | | | | | |
| | | D Like | | | | | | |
| 225572_at | CREB1 | CAMP Responsive | | I | 2 | 10.00 | 2 | 14.00 |
| | | Element Binding | | | | | | |
| | | Protein 1 | | | | | | |
| 236227_at | TMEM161B | Transmembrane | | I | 2 | 10.00 | 2 | 14.00 |
| | | Protein 161B | | | | | | |
| 209068_at | HNRNPDL | Heterogeneous | | I | 2 | 10.00 | 2 | 14.00 |
| | | Nuclear | | | | | | |
| | | Ribonucleoprotein | | | | | | |
| | | D Like | | | | | | |
| 202206_at | ARL4C | ADP Ribosylation | | I | 2 | 10.00 | 2 | 14.00 |
| | | Factor Like | | | | | | |
| | | GTPase 4C | | | | | | |
| 202724_s_at | FOXO1 | Forkhead Box O1 | | I | 2 | 10.00 | 2 | 14.00 |
| 1554678_s_at | HNRNPDL | Heterogeneous | | I | 2 | 10.00 | 2 | 14.00 |
| | | Nuclear | | | | | | |
| | | Ribonucleoprotein | | | | | | |
| | | D Like | | | | | | |
| 219387_at | CCDC88A | Coiled-Coil | I | I | 6 | 6.00 | 2 | 14.00 |
| | | Domain | | | | | | |
| | | Containing 88A | | | | | | |
| 212231_at | FBXO21 | F-Box Protein 21 | | I | 2 | 10.00 | 2 | 14.00 |
| 201993_x_at | HNRNPDL | Heterogeneous | | I | 2 | 10.00 | 2 | 14.00 |
| | | Nuclear | | | | | | |
| | | Ribonucleoprotein | | | | | | |
| | | D Like | | | | | | |
| 202741_at | PRKACB | Protein Kinase | | I | 2 | 10.00 | 2 | 14.00 |
| | | CAMP-Activated | | | | | | |
| | | Catalytic Subunit | | | | | | |
| | | Beta | | | | | | |
| 228708_at | RAB27B | RAB27B, Member | | I | 2 | 10.00 | 2 | 14.00 |
| | | RAS Oncogene | | | | | | |
| | | Family | | | | | | |
| 200595_s_at | EIF3A | Eukaryotic | | I | 2 | 10.00 | 2 | 14.00 |
| | | Translation | | | | | | |
| | | Initiation Factor 3 | | | | | | |
| | | Subunit A | | | | | | |
| 229414_at | PITPNC1 | Phosphatidylinositol | I | | 4 | 8.00 | 2 | 14.00 |
| | | Transfer | | | | | | |
| | | Protein, | | | | | | |
| | | Cytoplasmic 1 | | | | | | |
| 242230_at | ATXN1 | Ataxin 1 | I | | 4 | 8.00 | 2 | 14.00 |
| 211575_s_at | UBE3A | Ubiquitin Protein | I | | 2 | 10.00 | 2 | 14.00 |
| | | Ligase E3A | | | | | | |
| 202516_s_at | DLG1 | Discs Large | I | | 4 | 8.00 | 2 | 14.00 |
| | | MAGUK Scaffold | | | | | | |
| | | Protein 1 | | | | | | |
| 203165_s_at | SLC33A1 | Solute Carrier | I | | 4 | 8.00 | 2 | 14.00 |
| | | Family 33 | | | | | | |
| | | Member 1 | | | | | | |
| 211599_x_at | MET | MET Proto- | | D | 2 | 10.00 | 2 | 14.00 |
| | | Oncogene, | | | | | | |
| | | Receptor Tyrosine | | | | | | |
| | | Kinase | | | | | | |
| 240282_at | WDR1 | WD Repeat | | D | 2 | 10.00 | 2 | 14.00 |
| | | Domain 1 | | | | | | |
| 206669_at | GAD1 | Glutamate | | D | 2 | 10.00 | 2 | 14.00 |
| | | Decarboxylase 1 | | | | | | |
| 233293_at | NPAS3 | Neuronal PAS | | D | 2 | 10.00 | 2 | 14.00 |
| | | Domain Protein 3 | | | | | | |
| 224970_at | NFIA | Nuclear Factor I A | D | D | 4 | 8.00 | 2 | 14.00 |
| 244321_at | PGAP1 | Post-GPI | | D | 2 | 10.00 | 2 | 14.00 |
| | | Attachment To | | | | | | |
| | | Proteins 1 | | | | | | |
| 206706_at | NTF3 | Neurotrophin 3 | | D | 2 | 10.00 | 2 | 14.00 |
| 1569661_at | NPAS3 | Neuronal PAS | | D | 2 | 10.00 | 2 | 14.00 |
| | | Domain Protein 3 | | | | | | |
| 209749_s_at | ACE | Angiotensin I | | D | 2 | 10.00 | 2 | 14.00 |
| | | Converting | | | | | | |
| | | Enzyme | | | | | | |
| 205095_s_at | ATP6V0A1 | ATPase H+ | | D | 2 | 10.00 | 2 | 14.00 |
| | | Transporting V0 | | | | | | |
| | | Subunit A1 | | | | | | |
| 214667_s_at | TP53I11 | Tumor Protein | | D | 2 | 10.00 | 2 | 14.00 |
| | | P53 Inducible | | | | | | |
| | | Protein 11 | | | | | | |
| 228546_at | DPP6 | Dipeptidyl | | D | 4 | 8.00 | 2 | 14.00 |
| | | Peptidase Like 6 | | | | | | |
| 222420_s_at | UBE2H | Ubiquitin | | D | 4 | 8.00 | 2 | 14.00 |
| | | Conjugating | | | | | | |
| | | Enzyme E2 H | | | | | | |
| 210381_s_at | CCKBR | Cholecystokinin B | | D | 4 | 8.00 | 2 | 14.00 |
| | | Receptor | | | | | | |
| 206462_s_at | NTRK3 | Neurotrophic | | D | 4 | 8.00 | 2 | 14.00 |
| | | Receptor Tyrosine | | | | | | |
| | | Kinase 3 | | | | | | |
| 204724_s_at | COL9A3 | Collagen Type IX | | D | 4 | 8.00 | 2 | 14.00 |
| | | Alpha 3 Chain | | | | | | |
| 228237_at | PAPPA2 | Pappalysin 2 | | D | 4 | 8.00 | 2 | 14.00 |
| 1564525_at | GSN | Gelsolin | | D | 4 | 8.00 | 2 | 14.00 |
| 224976_at | NFIA | Nuclear Factor I A | | D | 4 | 8.00 | 2 | 14.00 |
| 217792_at | SNX5 | Sorting Nexin 5 | I | I | 4 | 6.00 | 4 | 14.00 |
| 201352_at | YME1L1 | YME1 Like 1 | | I | 4 | 6.00 | 4 | 14.00 |
| | | ATPase | | | | | | |
| 210285_x_at | WTAP | WT1 Associated | | I | 4 | 6.00 | 4 | 14.00 |
| | | Protein | | | | | | |
| 227449_at | EPHA4 | EPH Receptor A4 | | I | 4 | 6.00 | 4 | 14.00 |
| 204529_s_at | TOX | Thymocyte | | I | 4 | 6.00 | 4 | 14.00 |
| | | Selection | | | | | | |
| | | Associated High | | | | | | |
| | | Mobility Group | | | | | | |
| | | Box | | | | | | |
| 225314_at | OCIAD2 | OCIA Domain | | I | 4 | 6.00 | 4 | 14.00 |
| | | Containing 2 | | | | | | |
| 212704_at | ZCCHC11 | Zinc Finger CCHC- | | I | 2 | 8.00 | 4 | 14.00 |
| | | Type Containing | | | | | | |
| | | 11 | | | | | | |
| 202761_s_at | SYNE2 | Spectrin Repeat | | I | 2 | 8.00 | 4 | 14.00 |
| | | Containing | | | | | | |
| | | Nuclear Envelope | | | | | | |
| | | Protein 2 | | | | | | |
| 201523_x_at | UBE2N | Ubiquitin | | I | 2 | 8.00 | 4 | 14.00 |
| | | Conjugating | | | | | | |
| | | Enzyme E2 N | | | | | | |
| 218146_at | GLT8D1 | Glycosyltransferase | | I | 2 | 8.00 | 4 | 14.00 |
| | | 8 Domain | | | | | | |
| | | Containing 1 | | | | | | |
| 203944_x_at | BTN2A1 | Butyrophilin | | I | 2 | 8.00 | 4 | 14.00 |
| | | Subfamily 2 | | | | | | |
| | | Member A1 | | | | | | |
| 200023_s_at | EIF3F | Eukaryotic | | I | 2 | 8.00 | 4 | 14.00 |
| | | Translation | | | | | | |
| | | Initiation Factor 3 | | | | | | |
| | | Subunit F | | | | | | |
| 200016_x_at | HNRNPA1 | | | I | 2 | 8.00 | 4 | 14.00 |
| 213356_x_at | HNRNPA1 | | | I | 2 | 8.00 | 4 | 14.00 |
| 223010_s_at | OCIAD1 | OCIA Domain | | I | 2 | 8.00 | 4 | 14.00 |
| | | Containing 1 | | | | | | |
| 204160_s_at | ENPP4 | Ectonucleotide | I | | 4 | 6.00 | 4 | 14.00 |
| | | Pyrophosphatase/ | | | | | | |
| | | Phosphodiesterase | | | | | | |
| | | 4 (Putative) | | | | | | |
| 201992_s_at | KIF5B | Kinesin Family | I | | 2 | 8.00 | 4 | 14.00 |
| | | Member 5B | | | | | | |
| 201446_s_at | TIA1 | TIA1 Cytotoxic | I | | 2 | 8.00 | 4 | 14.00 |
| | | Granule | | | | | | |
| | | Associated RNA | | | | | | |
| | | Binding Protein | | | | | | |
| 223614_at | MMP16 | Matrix | | D | 2 | 8.00 | 4 | 14.00 |
| | | Metallopeptidase | | | | | | |
| | | 16 | | | | | | |
| 221792_at | RAB6B | RAB6B, Member | | D | 2 | 8.00 | 4 | 14.00 |
| | | RAS Oncogene | | | | | | |
| | | Family | | | | | | |
| 1566139_at | HOPX | HOP Homeobox | | D | 2 | 8.00 | 4 | 14.00 |
| 218293_x_at | NUP50 | Nucleoporin 50 | | D | 2 | 8.00 | 4 | 14.00 |
| 209270_at | LAMB3 | Laminin Subunit | | D | 4 | 6.00 | 4 | 14.00 |
| | | Beta 3 | | | | | | |
| 1564010_at | CAST | Calpastatin | | | 4 | 10.00 | 0 | 14.00 |
| 215210_s_at | DLST | Dihydrolipoamide | | I | 4 | 10.00 | 0 | 14.00 |
| | | S- | | | | | | |
| | | Succinyltransferase | | | | | | |
| 243683_at | MORF4L2 | Mortality Factor 4 | | I | 4 | 10.00 | 0 | 14.00 |
| | | Like 2 | | | | | | |
| 214559_at | DRD3 | Dopamine | | I | 4 | 10.00 | 0 | 14.00 |
| | | Receptor D3 | | | | | | |
| 202834_at | AGT | Angiotensinogen | I | | 4 | 10.00 | 0 | 14.00 |
| 200884_at | CKB | Creatine Kinase B | I | | 4 | 10.00 | 0 | 14.00 |
| 1568760_at | MYH11 | Myosin Heavy | | D | 4 | 10.00 | 0 | 14.00 |
| | | Chain 11 | | | | | | |
| 242832_at | PER1 | Period Circadian | | D | 4 | 10.00 | 0 | 14.00 |
| | | Clock 1 | | | | | | |
| 235952_at | DGKH | Diacylglycerol | | D | 4 | 10.00 | 0 | 14.00 |
| | | Kinase Eta | | | | | | |
| 201841_s_at | HSPB1 | Heat Shock | | D | 4 | 10.00 | 0 | 14.00 |
| | | Protein Family B | | | | | | |
| | | (Small) Member 1 | | | | | | |
| 207091_at | P2RX7 | Purinergic | | D | 4 | 10.00 | 0 | 14.00 |
| | | Receptor P2X 7 | | | | | | |
| 221354_s_at | MCHR1 | Melanin | | D | 4 | 10.00 | 0 | 14.00 |
| | | Concentrating | | | | | | |
| | | Hormone | | | | | | |
| | | Receptor 1 | | | | | | |
| 209072_at | MBP | Myelin Basic | | D | 4 | 10.00 | 0 | 14.00 |
| | | Protein | | | | | | |
| 211934_x_at | GANAB | Glucosidase II | | D | 4 | 10.00 | 0 | 14.00 |
| | | Alpha Subunit | | | | | | |
| 227090_at | PHF21A | PHD Finger | | D | 4 | 10.00 | 0 | 14.00 |
| | | Protein 21A | | | | | | |
| 200709_at | FKBP1A | FK506 Binding | | D | 4 | 10.00 | 0 | 14.00 |
| | | Protein 1A | | | | | | |
| 208817_at | COMT | Catechol-O- | | D | 4 | 10.00 | 0 | 14.00 |
| | | Methyltransferase | | | | | | |
| 200611_s_at | WDR1 | WD Repeat | | D | 4 | 10.00 | 0 | 14.00 |
| | | Domain 1 | | | | | | |
| 203421_at | TP53I11 | Tumor Protein | | D | 4 | 10.00 | 0 | 14.00 |
| | | P53 Inducible | | | | | | |
| | | Protein 11 | | | | | | |
| 202807_s_at | TOM1 | Target Of Myb1 | D | D | 4 | 10.00 | 0 | 14.00 |
| | | Membrane | | | | | | |
| | | Trafficking Protein | | | | | | |

TABLE 12
Drug repurposing- Connectivity Map for the longer
list of top candidate biomarkers CFE3 Score
of 14 and above. Direction of high mood.

| rank | cmap name | score |
|---|---|---|
| 1 | zalcitabine | 1 |
| 2 | mafenide | 0.986 |
| 3 | betazole | 0.949 |
| 4 | hesperidin | 0.946 |
| 5 | ritodrine | 0.935 |
| 6 | adiphenine | 0.927 |
| 7 | sirolimus | 0.925 |
| 8 | bumetanide | 0.924 |
| 9 | idoxuridine | 0.912 |
| 10 | adiphenine | 0.908 |
| 11 | thiamphenicol | 0.907 |
| 12 | atracurium | 0.895 |
| | besilate | |
| 13 | Prestwick-1083 | 0.89 |
| 14 | fludrocortisone | 0.879 |
| 15 | butamben | 0.876 |
| 16 | Prestwick-682 | 0.875 |
| 17 | sulfacetamide | 0.873 |
| 18 | isoflupredone | 0.868 |
| 19 | epirizole | 0.868 |
| 20 | salsolidin | 0.866 |
| 21 | heptaminol | 0.864 |
| 22 | protoveratrine A | 0.862 |
| 23 | droperidol | 0.861 |
| 24 | genistein | 0.858 |
| 25 | metformin | 0.852 |

TABLE 13
Drug repurposing- NIH LINCS for the longer list of top candidate
biomarkers CFE3 Score of 14 and above. Direction of high mood.

| score | Perturbation |
|---|---|
| 0.0758 | trichostatin A |
| 0.0682 | manumycin A |
| 0.0682 | AZD-7762 |
| 0.0606 | vorinostat |
| 0.0606 | BRD-K64606589 |
| 0.053 | Scriptaid |
| 0.053 | mitoxantrone |
| 0.053 | HDAC6 inhibitor ISOX |
| 0.053 | BRD-K64857848 |
| 0.053 | BRD-K91370081 |
| 0.053 | BRD-K84595254 |
| 0.0455 | Puromycin |
| | dihydrochloride |
| 0.0455 | KETOPROFEN |
| 0.0455 | Chemistry 2804 |
| 0.0455 | MENADIONE |
| 0.0455 | BRD-K56411643 |
| 0.0455 | BRD-K92317137 |
| 0.0455 | Importazole |
| 0.0455 | BRD-K04853698 |
| 0.0455 | PERHEXILINE |
| | MALEATE |
| 0.0455 | NCGC00188535-01 |
| 0.0455 | BRD-K60870698 |
| 0.0455 | NCGC00165188-01 |
| 0.0455 | BRD-K01896723 |
| 0.0455 | VU0418934-2 |

TABLE 14
Universal Predictor Mood (UP-Mood). Predictions using an apriori
algorithm combining as predictors BioM26 with mood (SMS7) and
with clinical severity of bipolar disorder (CFI-BP). In all subjects
in the independent test cohort. Cross-sectional analyses.

| Universal Predictor Mood (UP-Mood) | | |
|---|---|---|
| BioM26 + CFI-BP + SMS7) | Depression | Mania |
| State | AUC 69.1% | AUC 71.5% |
| Clinically Severe | P = 4.79E−04 | P = 5E−02 |
| Trait | OR 1.2 | OR 1.6 |
| All Future Hospitalizations | P = 1.57E−02 | P = 1.4E−03 |

While the present disclosure is amenable to various modifications and alternative forms, the methods that have been shown by way of example in the drawings are described in detail below. The intention, however, is not to limit the present disclosure to the any specific example. On the contrary, the present disclosure is intended to cover all modifications, equivalents, and alternatives falling within the scope of the present disclosure as defined by the appended claims.

1. A method for diagnosing and treating mood disorders in an individual in need thereof, comprising:
(a) measuring the expression levels and/or slope of biomarkers in a biological sample from an individual, wherein the biomarkers in a first panel of biomarker comprise one or more of: TMEM161B, GLO1, PRPS1, SMAD7, ANK3, OGT, CD47, GLS, TMEM106B, RPL3, FANCF, HNRNPDL, DOCK10, or CALM1, and
wherein the biomarkers in a second panel of biomarkers comprise one or more of: NRG1, OLFM1, SPECC1, SORT1, TPH1, GSK3B, MARCKS, NR3C1, and SLC6A4;
(b) comparing the expression level or slope of each biomarker measured in the sample to the expression level and/or slope of a matched biomarker determined in a clinically relevant population;
(c) generating a score for the individual, wherein the score is determined by summing:
the number of biomarkers in the first panel of biomarkers exhibiting changed expression and/or slope relative to the expression level and/or slope in the matched biomarkers determined in the clinically relevant population and
the number of biomarkers in the second panel of biomarkers expressing changed expression and/or slope relative to the expression level and/or slope in the matched biomarkers determined in the clinically relevant population
(d) diagnosing the individual as having a mood disorder, and/or an increased risk for developing a mood disorder risk based on the difference between the scores of the individual and the scores of the matched biomarkers in the clinically relevant population; and
(e) treating the individual diagnosed with a mood disorder, and or the individual diagnosed with an increased risk for developing a mood disorder.

2. The method of claim 1, wherein the treating step includes treating the individual diagnosed with mood disorder and/or diagnosed with an increased risk for developing a mood disorder with a treatment consistent with clinical practice guidelines.

3. The method of claim 1, wherein the treating step includes providing the individual with at least one therapeutic compound known to treat mood disorders.

4. The method of claim 1, wherein the treating step includes providing the individual with at least one therapeutic compound which is a repurposed compound.

5. The method of claim 1, further including the steps of:
monitoring the individual to determine if the treatment is efficacious, wherein the monitoring step includes obtaining at least one addition biological sample from the individual;
determining the score of the at least one additional biological sample from the individual; and
and comparing the scores of the at least one additional biological sample to the scores of the individual determined before and after or during treatment.

6. The method of claim 1, wherein the mood disorder is selected from the group consisting of depression or bipolar disorder.

7. The method of claim 1, wherein the score is determined by assigning a weighted coefficient to each biomarker based on the importance of each biomarker in assessing and predicting mood disorders and an increase in risk of developing a mood disorder.

8. The method of claim 1, wherein the biological sample is a tissue sample or a fluid, such as cerebrospinal fluid, whole blood, blood serum, plasma, saliva, or other bodily fluid, or an extract, fraction, or purification product thereof.

9. The method of claim 1, wherein the biomarker expression level of the biomarker is determined in the biological sample by measuring a level of biomarker RNA or protein.

10. The methods of claim 1, wherein the individual is treated with at least one compound selected from the list comprising: lithium, valproic acid, and other mood stabilizers; amoxapine, paroxetine, mirtazapine, buspirone, fluoxetine, amitriptyline, nortriptyline, trimipramine, and other antidepressants; clozapine, chlorpromazine, haloperidol, paliperidone, iloperidone, asenapine, cariprazine, lurasidone, quetiapine, olanzapine, risperidone, aripiprazole, brexpiprazole, and other antipsychotics; docosahexaenoic acid and other omega-3 fatty acids; diazepam and other anxiolytics; ketamine and other dissociants; and CBT or other psychotherapy treatments.

11. The methods of claim 1, wherein:
(a) the individual exhibiting changes in one or more of biomarkers: NRG1, PRPS1, CD47 is treated with at least one mood stabilizing compound;
(b) the individual exhibiting changes in one or more of biomarkers: SLC6A4, DOCK10, NRG1, CD47 is treated with at least one antidepressant compound;
c) the individual exhibiting changes in one or more of biomarkers: GLO1, SLC6A4, CD47, GLS, HNRNPDL, is treated with at least one of the following compounds: docosahexaenoic acid and other omega-3 fatty acids; and
(d) the individual exhibiting changes in one or more of biomarkers: NRG1, CD47, GLS, is treated with at least one antipsychotic compound.

12. The method of claim 1, wherein the treating step includes administering to the individual at least one compound selected from the group consisting of: an isoflupredone, trichostatin A, dubinidine, ciprofibrate, pioglitazone, tropine, an adiphenine, saquinavir, chlorogenic acid, pindolol, lansoprazole, xamoterol, methanthelinium bromide, asiaticoside, an estradiol, methacholine, carteolol, chlorcyclizine, atracurium besylate, Chicago Sky Blue 6B, enoxacin, a levobunolol, 15-delta prostaglandin J2, pirinixic acid, NNC 55-0396 dihydrochloride, nadolol, MLN4924, U0126, amcinonide, iopanic acid, rosuvastatin and therapeutically acceptable salts thereof.

13. The method of claim 1, wherein the individual is diagnosed with depression,
when
(a) the expression levels of at least one of the biomarkers in the first panel of biomarkers comprising TMEM161B, GLO1, PRPS1, SMAD7, CD47, GLS, FANCF, HNRNPDL, and DOCK10, in the biological sample of the individual are decreased relative to the expression level of matched biomarkers determined in a clinically relevant population; and
(b) the expression levels of at least one of the biomarkers in the second panel of biomarkers comprising NRG1, OLFM1, and SLC6A4, in the biological sample of the individual is increased relative to the expression level of matched biomarkers determined in a clinically relevant population.

14. The method of claim 9, wherein the therapeutic is one or more of a repurposed drug selected from the group consisting of: an isoflupredone, trichostatin A, dubinidine, ciprofibrate, pioglitazone, tropine, an adiphenine, saquinavir, chlorogenic acid, pindolol, lansoprazole, xamoterol, methanthelinium bromide, asiaticoside, an estradiol, methacholine, a carteolol, chlorcyclizine, NNC 55-0396 dihydrochloride, nadolol, MLN4924, U0126, amcinonide, iopanic acid, and rosuvastatin.

15. The method of claim 1, wherein when the individual is diagnosed with bipolar depression
when
(a) the expression levels of at least one of the biomarkers in the first panel of biomarkers comprising TMEM161B, PRPS1, GLS, RPL3, and DOCK10, in the biological sample of the individual, are decreased relative to the expression level of matched biomarkers determined in a clinically relevant population, and
(b) the expression levels of at least one of the biomarkers in the second panel of biomarkers comprising NRG1, and SLC6A4, in the biological sample of the individual is increased relative to the expression level of matched biomarkers determined in a clinically relevant population.

16. The method of claim 11, wherein the therapeutic is one or more of a new method of use/repurposed drugs selected from the group consisting of: atracurium besylate, Chicago Sky Blue 6B, enoxacin, levobunolol, 15-delta prostaglandin J2, ciprofibrate, pirinixic acid, an isoflupredone, and trichostatin A.

17. A method for monitoring response to treatment of a mood disorder and determining treatment efficacy in an individual, comprising the steps of:
(a) measuring an expression levels of biomarkers in at least 2 biological samples from the individual and comparing the measured expression levels to an expression level of a matched biomarker determined in a clinically relevant population, wherein the at least one biomarker is from a first panel, comprising: TMEM161B, GLO1, PRPS1, SMAD7, ANK3, OGT, CD47, GLS, TMEM106B, RPL3, FANCF, HNRNPDL, DOCK10, and CALM1, and/or
wherein the at least one biomarker is from a second panel comprising: NRG1, OLFM1, SPECC1, SORT1, TPH1, GSK3B, MARCKS, NR3C1, and SLC6A4, wherein the expression level of the one or more biomarkers in the biological sample is changed, and wherein at least one of the at least two biological samples is collected before the individual is treated for a mood disorder and at least one of the at least two biological samples is collected after the individual is treated for a mood disorder;
(b) calculating a score for the biomarkers in the biological samples, by summing:
the number of biomarkers in the first panel exhibiting a change in expression level relative to the expression of the biomarker determined in a clinically relevant population, and/or
the number of biomarkers in the second panel exhibiting a change in expression level relative to the expression of the biomarker determined in a clinically relevant population; and
(c) determining that said treatment(s) is effective if the score of the panel of biomarker(s) in the sample collected after treatment is lower than the score of at least one of the at least two biologicals samples collected before treatment.

18. A method of assessing and treating mood disorders in an individual in need thereof, comprising:
calculating combined biomarkers and clinical information Up-Mood Score based on the equation:
(Biomarker Panel Score)+(Clinical Risk Score)+(Mood Score)=Up-Mood Score;
wherein the Biomarker Panel Score is obtained as per the method of claim 1;
wherein the Clinical Risk Score is calculated by summing up clinical risk factors of severity of illness;
wherein the Mood Score is calculated by using a mood-rating scale assessing the level of mood disorder of the individual by comparing the individual's Up-Mood Score to a reference Up-Mood Score;
administering a treatment for mood to the individual when the individual's Up-Mood Score is different than a reference Up-Mood Score; and
monitoring the individual's response to a treatment for mood by determining changes in the Up-Mood Score after initiating a treatment.

19. (canceled)

20. (canceled)

21. The method of claim 1, wherein the biomarkers include at least one of the following biomarkers: NRG1, SLC6A4, DOCK10, or combinations thereof, and are used in all individuals.

22. The method of claim 1, wherein the biomarkers include at least one of the following biomarkers: NRG1, SLC6A4, DOCK10, MARCKS, or combinations thereof, and are used in males.

23. The method of claim 1, wherein the biomarkers include at least one of the following biomarkers: NRG1, SLC6A4, GLS, PRPS1, ANK3, or combinations thereof, and are used in females.

24. The method of claim 1, wherein the biomarkers include at least one of the following biomarkers: MARCKS, SLC6A4, or combinations thereof, and are used in males with bipolar disorder.

25. The method of claim 1, wherein the biomarkers include at least one of the following biomarkers: TMEM106B, SMAD7, ANK3, SORT1, PRPS1, DOCK10, or combinations thereof, are used in females with bipolar disorder.

26. The method of claim 1, wherein the biomarkers include at least one of the following biomarkers: NRG1, CD47, MARCKS, NR3C1, SLC6A4, or combinations thereof, are used in males with depression.

27. The method of claim 1 wherein the biomarkers include at least one of the following biomarkers: GSK3B, OLFM1, OGT, or combinations thereof, are used in females with depression.

28. The method of claim 1, wherein the biomarkers include at least one of the following biomarkers: NRG1, SLC6A4, or combinations thereof, are used in males with PTSD.

29. The method of claim 1, wherein the biomarkers include at least one of the following biomarkers: NRG1 is used in females with PTSD.

30. The method of claim 1, wherein the biomarkers include at least one of the following biomarkers: PRPS1, CALM1, SPECC1, TPH1, DOCK10, OLFM1, MARCKS, RPL3, NRG1, GSK3B, GLS, or combinations thereof, are used in males with psychotic disorders.

31. The method of claim 1, wherein the biomarkers include at least one of the following biomarkers: MARCKS, RPL3, or combinations thereof, are used in females with psychotic disorders.
